(19) United States
(12) Patent Application Publication
(10) Pub. No.: US 2024/0402187 A1
(43) Pub. Date: Dec. 5, 2024

(54) GLYCAN AGE PREDICTION MODEL

(71) Applicant: The Regents of the University of California, Oakland, CA (US)

(72) Inventors: Emanual Maverakis, Sacramento, CA (US); Alexander Merleev, Sacramento, CA (US); Carlito Lebrilla, Davis, CA (US)

(73) Assignee: The Regents of the University of California, Oakland, CA (US)

(21) Appl. No.: 18/700,666

(22) PCT Filed: Oct. 14, 2022
(86) PCT No.: PCT/US2022/046769

§ 371 Date: Apr. 11, 2024

Related U.S. Application Data
(60) Provisional application No. 63/255,850, filed on Oct. 14, 2021.

(51) Int. Cl. G01N 33/68

(52) U.S. Cl. CPC G01N 33/6848; G01N 2440/38

(57) ABSTRACT

Provided herein are methods for determining the age of a subject by measuring the relative abundance of glycopeptides (e.g., using mass spectrometry) in a biological sample from the subject. Also provided are methods for comparing the relative abundance of the glycopeptides to age prediction models to determine the age of the subject. The age prediction models provided herein are based on the relative abundance of the glycopeptides in control populations.

### CROSS-REFERENCE TO RELATED APPLICATIONS

The present application claims priority to U.S. Provisional Application No. 63/255,850 filed Oct. 14, 2021, the full disclosure of which is incorporated by reference in its entirety for all purposes.

### BACKGROUND

Aging is a complex and ubiquitous biological process that leads to accumulation of molecular, cellular, and organ damage, resulting in reduced health, increased vulnerability to disease, and eventually to death. The chronological and biological age of individuals can vary. For example, lifestyle choices such as smoking may increase the rate of biological aging relative to chronological aging. While various biomarkers have been used to estimate biological age, there remains a need for accurate and easily measured biomarkers for determining the age of a subject using a biological sample.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based in part on the novel application of mass spectrometry to measure glycopeptides in biological samples, as well as the finding that chronological age correlates strongly with the relative abundance of one or more measured glycopeptides.

In one aspect, provided herein are methods for determining the age of a biological sample from a subject. In some embodiments, the age of the subject is determined based on the age of the biological sample. In some embodiments, the methods comprise measuring a relative abundance of at least one glycopeptide in the biological sample. In some embodiments, the at least one glycopeptide comprises any of the glycopeptides in Table 2 herein. In some embodiments, the at least one glycopeptide comprises IgG1-3510, IgG1-5410, IgM-209-5411, IgM-J-5412, Haptoglobin (Hp)-241-7602, or a combination thereof. In some embodiments, the at least one glycopeptide comprises IgG1-3510, IgG1-5410, IgG2-3410, IgM-209-5411, IgM-J-5412, Hp-241-7602, or a combination thereof.

In some embodiments, the methods herein further comprise measuring a concentration of at least one protein in the biological sample. In some embodiments, the at least one protein comprises any of the proteins in Table 2. In some embodiments, the at least one protein comprises IgG3.

In some embodiments, the methods comprise comparing the relative abundance of the at least one glycopeptide and/or the concentration of the at least one protein to an age prediction model, wherein the age prediction model comprises the relative abundance of the at least one glycopeptide and/or the concentration of the at least one protein in at least one control biological sample. In some embodiments, each control biological sample is from a control individual of a known age. In some embodiments, the age prediction model comprises the relative abundance of the at least one glycopeptide in a plurality of control biological samples. In some embodiments, the age prediction model comprises a linear regression model or a multiple linear regression model based on a correlation between the relative abundance of the at least one glycopeptide in the at least one control biological sample and the age of the control individual. In some embodiments, the age prediction model comprises one of the multiple linear regression models of Table 5 herein.

In some embodiments, the biological samples and the control biological samples are liquid samples. In some embodiments, the samples are blood samples, serum samples, plasma samples, or a combination thereof.

In some embodiments of the methods herein, measuring the relative abundance of at least one glycopeptide and/or measuring the concentration of at least one protein comprises mass spectrometry (e.g., multiple reaction monitoring mass spectrometry). In some embodiments, measuring the relative abundance of the at least one glycopeptide comprises calculating the relative response of the at least one glycopeptide as the area under the mass spectrometry curve of the at least one glycopeptide divided by the area under the curve of a non-glycosylated reference peptide from the same protein as the at least one glycopeptide.

In some embodiments, the subject is male or female. In some embodiments, the biological sample is from a criminal forensics investigation.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows a site-specific map for several exemplary glycopeptides, according to aspects of this disclosure. Blue square: N-acetylglucosamine; green circle: mannose; yellow circle: galactose; red triangle: fucose; purple diamond: N-acetylneuraminic acid; yellow square: N-acetylgalactosamine.

FIG. 2 shows a site-specific map of the most common glycan modifications of the most common serum glycoproteins (excluding immunoglobulins), according to aspects of this disclosure. Putative structures and locations are shown for the site-specific glycans that were monitored in the study described in the Examples herein. Blue square: N-acetylglucosamine; green circle: mannose; yellow circle: galactose; red triangle: fucose; purple diamond: N-acetylneuraminic acid; yellow square: N-acetylgalactosamine. The structures represent the most common glycans occurring at each glycosylation site. Some glycosylation sites can be expressed without a modifying glycan, in which case the non-glycosylated version was also monitored. For each protein, a non-glycosylated reference peptide, bolded sequence, present across all glycoforms was used to calculate the relative abundance of each glycoform (i.e. area under the curve of the glycoform divided by the area under the curve of the non-glycosylated reference peptide).

FIG. 3 shows a site-specific glycan map for the Immunoglobulins (Igs), according to aspects of this disclosure. The CH2 84.4 Ig glycosylation site is conserved across all IgG subclasses (IgG1-4). Glycans at this site and other sites across the different Ig classes (IgA, IgG, IgM, and J chain) were monitored. To provide the relative abundance of each IgG subclass IgG1-4) the abundance of subclass-specific non-glycosylated peptides were calculated relative to a single non-glycosylated peptide common to all IgG subclasses (IgG1-4). In addition, glycosylated peptides within each subclass were determined relative to a non-glycosylated peptide common to all glycoforms. For IgG3 and IgG4 the glycosylated peptides amino acid sequence was identical, so the two similar Ig subclasses could not be distinguished. Thus, glycosylated peptides from this region are referred to as IgG3/4. Blue square: N-acetylglucosamine; green circle: mannose; yellow circle: galactose; red triangle: fucose; purple diamond: N-acetylneuraminic acid; yellow square: N-acetylgalactosamine.

FIG. 4 shows a site-specific map of the human serum glycome, according to aspects of this disclosure. The major glycans occurring at the glycosylation sites of the 17 most common serum glycoproteins are presented. When present, the sites of glycosylation (first of the two numbers) are as indicated in UNIPROT. When there is no position indicated, the glycosylation occurs at the immunoglobulin constant heavy chain domain 2 (CH2)-84.4 glycosylation site (IMGT numbering system). Glycan structures are presented as a four-digit code where the first numeral represents the total number of mannose and galactose residues combined, the second represents the total number of N-acetylglucosamine residues, the third numeral corresponds to the number of fucose residues, and the final numeral is the number of sialic acid moieties. On the right side of each diagram is the log of the relative abundances of the glycans presented as box-and-whisker plots. The left and right bars connected to each box indicate the boundaries of the normal distribution and the left and right box edges mark the first and third quartile boundaries within each distribution. The bold line within the box indicates the median value of the distribution. On the left of each diagram are the square of the intra-protein Pearson Product Moment Correlation Coefficients (PPMCCs) for connected glycan pair.

FIG. 5 shows intra-and inter-protein glycan associations, according to aspects of this disclosure. Log relative abundances for individual glycan pairs were graphed, and correlations were determined using Pearson Product Moment Correlation Coefficients (PPMCCs), which is abbreviated as “r”. (A to D) are intra-protein correlations. (E) represents inter-protein glycan correlations. (F) represents protein-glycan correlations.

FIG. 6 shows site-specific inter-protein and intra-protein glycan associations, according to aspects of this disclosure. To visualize the 16,742 correlations that were made, a machine learning dimensionality reduction strategy, t-Distributed Stochastic Neighbor Embedding, was used. Individual glycosylation sites are represented as distinct symbols. Each copy of the symbol represents a unique glycan occurring at that site. The distance between any two symbols represents the strength of the glycan pair's Pearson Product Moment Correlation Coefficient such that strongly correlating glycans are located close to each other. From this diagram it is apparent that there are both intra-protein and inter-protein glycan correlations. In addition, correlations are grouped into clusters indicating that not all glycosylation sites within a protein correlate with one another.

FIG. 7 shows the effect of age and gender on glycosylation, according to aspects of this disclosure. (A) Log relative glycan abundance versus age. Examples of glycoforms significantly altered by age (a full list can be found in Table 2). Of note, IgG1 and IgG2 share several age-associated glycan modifications. Also, glycan 5411 is negatively correlated with age when present on IgG1, IgG2, and position 209 of IgM. IgM also declines with increasing age (P=0.0011). (B) Representative site-specific glycosylations and proteins that are differentially expressed with respect to gender (a full list can be found in Table 3). The upper and lower bars connected to each box indicate the boundaries of the normal distribution and the upper and lower box edges mark the first and third quartile boundaries within each distribution. The bold line within the box indicates the median value of the distribution. Y-axis represents log relative abundance or log protein concentration where indicated.

FIG. 8 shows age and gender distribution of participants in the study described in the Examples herein. (A) Histogram of age distribution for healthy controls. (B) Box plot of age distribution by gender within the healthy control group.

FIG. 9 shows a meta-analysis of glycan associations with age, according to aspects of this disclosure. Forest plots were generated to estimate the Pearson Product Moment Correlation Coefficients (which is abbreviated as “r”) between the relative abundances of the indicated glycans and age. In these plots the confidence interval for each dataset is represented by the horizontal lines and the area of each square is proportional to the study's weight in the metanalysis. The final random effects models (RE model) represent the weighted average of the glycan correlations across the different independent data sets and 95% confidence intervals are provided for the given glycan's correlation with age. In each presented case, the confidence interval did not cross zero, although in 4 out of the 12 cases (IgA 1/2 p:144 g:5402, IgG2 g:3510,IgG2 g:5411, and IgM p:209 g:5412) the residual heterogeneity was significant, meaning that the variation in glycan age correlations between datasets was high.

FIG. 10 shows a meta-analysis of glycan associations with gender, according to aspects of this disclosure. Forest plots were generated to estimate the relative abundance of the indicated glycans or proteins across gender. In each case a final Random effects model (RE model) was constructed to represent the weighted average and 95% confidence interval for a given glycan's abundance. In each presented case the confidence interval did not cross zero and in all cases the residual heterogeneity was not statistically significant. In these plots the confidence interval for each dataset is represented by the horizontal lines and the area of each square is proportional to the study's weight in the meta-analysis.

FIG. 11 shows age prediction models, according to aspects of this disclosure. (A) The graph represents the performance of a linear regression model for age prediction. The model was constructed from 5 different glycopeptides (IgG1 g:3510, IgG1 g:5410, IgM p:209 g:5411, IgM J chain g:5412, Hp p:241 g:7602). Diagnostic plots (residuals vs fitted, testing for linearity; normal Q-Q, to assess the distribution of the residuals; scale-location, to assess the homoscedastic of the data; and residuals vs leverage, to check for overly influential cases) for the model are presented to its right. (B) Linear regression model comprised of six glycopeptides (IgG1 g:3510, IgG1 g:5410, IgG2 g:3410, IgM p:209 g:5411, IgM J chain g:5412, Hp p:241 g:7602) and 1 serum protein, IgG3. Model diagnostics are represented to the right (model performance parameters for age prediction models can be found in Table 5).

FIG. 12 shows performance of age models with differing number of predictors (n), according to aspects of this disclosure. (A) Linear regression model performance improved with incorporation of additional glycans until 5 glycans were incorporated. (B) The performance of the linear regression model comprised of both glycoforms and serum protein concentrations improved until 7 analytes were incorporated. n=7 was chosen as the final model.

FIG. 13 shows dynamic multiple reaction monitoring mass spectrometry (MRM MS) data, according to aspects of this disclosure. Spectra generated by QqQ mass spectrometry are shown. The MRM MS technique is dependent on predetermined knowledge of each glycopeptide's retention time and its collision-induced dissociation (CID) pattern (Table 1). The development of the annotated libraries containing this information have been well described (17,35,36). Knowledge of the CID pattern and analyte retention time allows for single transition monitoring of over 1000 specific glycopeptides. Representative compounds are shown.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Terminology

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e. at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of”' (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et la. “Adding amino acids to the genetic repertoire,” 9(6): 548-54 (2005)); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

### II. Introduction

Provided herein are methods for measuring and using the relative abundance of glycopeptides in biological samples from subjects to estimate the age of the subjects. As demonstrated herein, glycopeptides can be efficiently and accurately measured in biological samples, and the relative abundances of certain glycopeptides correlate strongly with chronological age. Along with nucleic acids, proteins, and lipids; glycans (oligosaccharides) are one of the four fundamental classes of molecules that make up all living systems (1). Traditionally, the information stream of a cell is viewed as starting in the genome and ending with a set of expressed proteins, representing the cell's phenotype. However, in order for a protein to function appropriately, it often requires post-translational modifications, of which glycans are one of the most commonly added modifiers. They can function as protein “on and off” switches or as “analog regulators” to fine-tune and direct protein function (2). The process that synthesizes and enzymatically attaches glycans to organic molecules is called glycosylation and it can produce thousands of unique glycan structures by linking together a finite set of sugar monomers (3). However, unlike DNA, RNA and protein synthesis, there is no template to guide the production of glycans. The process is thus immensely complex and impossible to predict from gene expression profiles alone. In fact, when one considers the massive 3-dimensional structural diversity of glycans combined with their variation in attachment sites, the complexity of the glycome parallels that of the genome (2).

As part of their glycoscience “Roadmap” (2), the National Research Council of the U.S. National Academies highlighted the importance of developing a site-specific map of the serum glycome, which would aid in the development of glycans as biomarkers of human diseases. One reason for the excitement around the use of glycans as disease-specific biomarkers is that glycosylation is a process influenced by a variety of factors including: the type of cell and its activation state; environmental factors, such as the presence of available metabolites; the age of the cell, as glycan moieties can be lost over time; and inflammatory mediators, such as cytokines and chemokines. All these factors can be altered in the setting of human diseases, making the glycome an expression of the overall health status of an individual. Furthermore, it has been hypothesized that glycans not only become altered in the setting of human disease but that they actually play a major role in the etiology of all human diseases (2). It is therefore not surprising that alterations in the glycome have already been linked to a variety of human diseases, especially cancer and autoimmunity (4-16). Most of these prior studies used labor-intensive methodologies to characterize glycans released from purified proteins and perhaps for this reason, detailed analyses have only been conducted on a relatively small number of patients. Lower resolution techniques, which yield limited structural information or no site-specific information, have been used to characterize larger patient cohorts, but such analyses are not ideally suited for biomarker discovery research. As a result, the sensitivity and specificity of site-specific glycosylations as disease-specific multi-analyte classifiers of autoimmunity is currently unknown.

In comparison to the advances made in the fields of genomics and proteomics, glycoscience remains relatively understudied, which is due to a lack of the analytical tools needed to drive the field forward (2). In this regard, glycoscience is similar to where the field of genetics was during the initial stages of the human genome project (2). Mass spectrometry (MS)-based technologies remain very appealing for glycan biomarker research because glycans are ionizable molecules. Also, the potential to accurately profile and quantitate thousands of glycan structures from a relatively small amount of starting material (e.g. 2 μl of serum) makes glycans superior to other molecules traditionally used as biomarkers of human diseases. For example, a site-specific glycoprofiling method could theoretically increase the accuracy of a serum protein biomarker by subdividing it into its different glycoforms.

With the goal of deploying glycan biomarkers clinically, Multiple Reaction Monitoring (MRM) has been developed to site-specifically characterize the human glycome in a rapid and reproducible fashion (17). Although MRM MS is mainly used in the fields of metabolomics and proteomics (18-21), its high sensitivity and linear response over a wide dynamic range makes it especially suited for glycan detection (22). In the studies described herein, MRM MS is used to construct a detailed site-specific structural map of the human plasma glycome of healthy individuals and to characterize the glycans' inter-and intra-molecular correlations. Glycan alterations associated with age and gender (common covariants in biomarker research and discovery) were also identified and multi-analyte classifiers capable of predicting age were constructed and validated.

### III. Age Determination Methods

In one aspect, provided herein is a method for determining the age of a biological sample from a subject. As used herein, the term “subject” refers to animals such as mammals, including, but not limited to, humans, non-human primates, cows, sheep, goats, horses, dogs, cats, rabbits, rats, mice and the like. In some embodiments, the biological samples used in the methods provided herein are obtained from a human subject. In some embodiments, the subject is male or female. In some embodiments, the biological samples are obtained as part of a forensics investigation (e.g., criminal forensics). As used herein, the term “age” and its grammatical equivalents may refer to either chronological age, i.e., the length of time that a living organism has been alive, or biological age (also referred to as physiological age), i.e., how old the body of a living organism seems to be, based on any of a number of biological factors. The methods herein may be used to determine or predict chronological age, biological age, or both chronological age and biological age.

A biological sample of the present disclosure may be any suitable sample from a subject (e.g., a solid sample, a liquid sample, a tissue sample, a cellular sample, a waste sample, etc.). In some embodiments, the sample is a blood sample. In some embodiments, the blood sample is a whole blood sample. In some embodiments, the whole blood sample is processed (e.g., by centrifugation or filtration) to enrich one or more blood components. In some embodiments, the blood sample has been processed to deplete one or more blood components. In some embodiments, the blood sample comprises plasma, serum, buffy coat, or any other blood fraction. In some embodiments, the blood sample comprises venous and/or capillary blood. In some embodiments, the biological sample is a blood sample, a serum sample, a plasma sample, or a combination thereof.

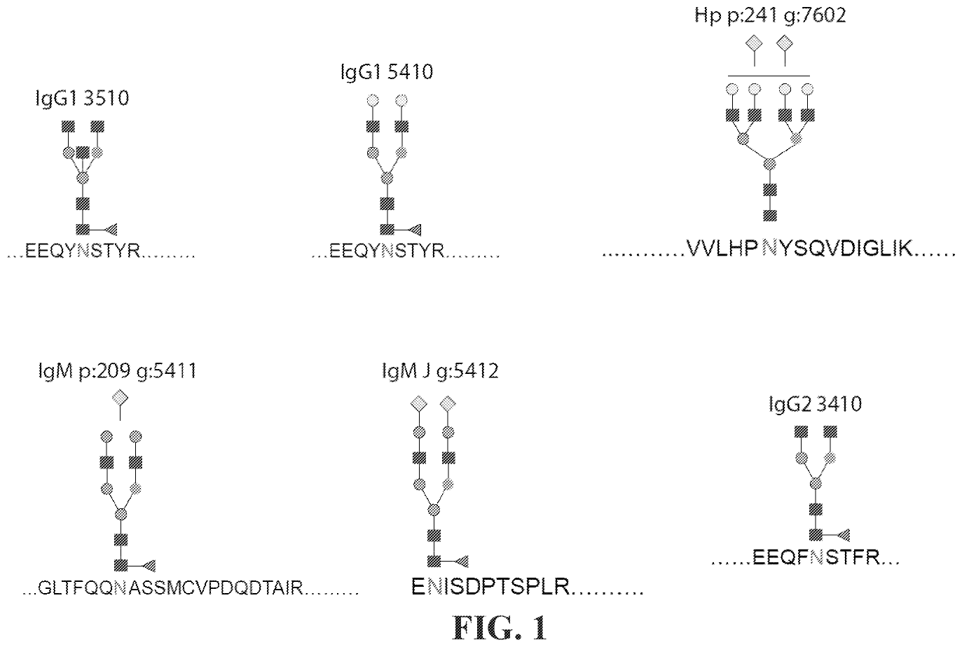

In some embodiments, the methods provided herein comprise measuring a relative abundance of at least one glycopeptide (e.g., one glycopeptide, two glycopeptides, three glycopeptides, four glycopeptides, five glycopeptides, six glycopeptides, seven glycopeptides, eight glycopeptides, nine glycopeptides, ten glycopeptides, or more) in a biological sample. In some embodiments, the at least one glycopeptide comprises any of the glycopeptides in Table 2. In some embodiments, the at least one glycopeptide comprises at least one (e.g., one, two, three, four, five, or all six) of the glycopeptides shown in FIG. 1. In some embodiments, the at least one glycopeptide comprises at least one (e.g., one, two, three, four, or all five) of IgG1-3510, IgG1-5410, IgM-209-5411, IgM-J-5412, Haptoglobin (Hp)-241-7602. In some embodiments, the at least one glycopeptide comprises at least one (e.g., one, two, three, four, five, or all six) of IgG1-3510, IgG1-5410, IgG2-3410, IgM-209-5411, IgM-J-5412, Hp-241-7602.

In the present disclosure, glycopeptides are designated using the format [protein]-[glycosylation site (optional)]-[glycan structure]. The protein is generally indicated using the common name (e.g., as indicated in UNIPROT), but abbreviations and/or alternative names may be used as indicated. When present, the glycosylation site (e.g., the amino acid residue to which the glycan structure is connected) is indicated following UNIPROT numbering. When there is no position indicated, the glycosylation occurs at the immunoglobulin constant heavy chain domain 2 (CH2)-84.4 glycosylation site (IMGT numbering system). Glycan structures are presented as four-digit codes. The first digit represents the total number of hexose sugars (e.g., the number of mannose and galactose residues combined); the second digit represents the total number of N-acetylglucosamine residues; the third digit represents the number of fucose residues; and the fourth digit represents the number of sialic acid moieties. In some embodiments (e.g., in humans), sialic acid is N-acetylneuraminic acid (Neu5Ac or NANA). As an example, Hp-241-7602 refers to haptoglobin (protein name) with a glycan at residue 241 (glycosylation site) having 7 hexose sugar residues, 6 N-acetylglucosamine residues, 0 fucose residues, and 2 sialic acid residues.

In the present disclosure, glycopeptides and glycans may also be depicted schematically (e.g., in FIGS. 1-3 and Table 8 herein). In such depictions, shapes and colors are used to indicate glycan residues. Unless indicated otherwise, a blue square represents N-acetylglucosamine; a green circle represents mannose; a yellow circle represents galactose; a red triangle represents fucose; a purple diamond represents sialic acid (e.g., N-acetylneuraminic acid); and a yellow square represents N-acetylgalactosamine. In such depictions, peptide sequences of the protein may also be indicated using the standard 1 letter IUPAC code. Such peptide sequences may show the whole protein sequence or only a portion of the protein sequence. The residue number of one or more amino acid residues may also be indicated in the depiction according to the UNIPROT protein numbering scheme. In some embodiments, the schematic depictions of glycopeptide structures show the most likely connectivity of the constituent glycan residues. However, it will be understood that other connective structures are possible. As such, any schematic depiction of one or more glycan residues is intended to represent any possible combination of connections between the residues shown.

Various methods may be used to measure the relative abundance of the glycopeptides described herein. In some embodiments, the methods comprise a mass spectrometry (MS) technique. In some embodiments, the methods comprise multiple reaction monitoring mass spectrometry (MRM MS). In some embodiments, the methods comprise isolating the biological sample (e.g., serum or plasma) from a subject. In some embodiments, the methods comprise digesting the proteins in the biological sample (e.g., with trypsin), which creates a mixture of peptides and glycopeptides. In some embodiments, measuring the relative abundance of a glycopeptide (or a peptide) comprises calculating the relative response of each glycopeptide as the MS area under the curve of the glycopeptide divided by the MS area under the curve of a non-glycosylated reference peptide from the same protein. This is different from absolute protein concentrations, which is determined by a calibration curve (also called a standard curve). To create the calibration curve, standard proteins are digested with trypsin and a dilution series is made. The dilution series is then analyzed by mass spectrometry.

In some embodiments, the methods provided herein comprise comparing the relative abundance of at least one glycopeptide to an age prediction model. In some embodiments, the age prediction model comprises the relative abundance of the at least one glycopeptide in at least one (e.g., at least two, at least three, at least five, at least 10, at least 20, at least 50, at least 75, at least 100, or more) control biological sample(s), wherein each control biological sample is from a control individual of a known age, thereby determining the age of the biological sample. In some embodiments, the age of the subject is determined based on the age of the biological sample. In some embodiments, the age prediction model comprises the relative abundance of the at least one glycopeptide in a plurality of control biological samples. In some embodiments, a control population of individuals of different ages is used to identify glycopeptides that are associated with age. For example, for each glycopeptide, a scatter plot may be created by plotting the relative abundance of the glycopeptide against age for each control individual. From this scatter plot, a correlation coefficient and p value may be calculated. In some embodiments, a control population of individuals comprises individuals of any age. For example, a control population may be selected to represent the general age distribution of a larger population (e.g., the population the subject of interest is part of).

In some embodiments, the age prediction model comprises a linear regression model or a multiple linear regression model based on a correlation between the relative abundance of the at least one glycopeptide in the at least one control biological sample and the age of the control individual. For example, a single or multiple glycopeptide age prediction classifier (i.e., an age prediction model) may be constructed from the glycopeptides that correlate with age (e.g., as described above). Such an age prediction model can be represented as [Age=X1G1+X2G2 . . . XnGn+C], where X1, X2 . . . Xn represent coefficients G1, G2 . . . Gn represent glycopeptide abundance, and C represents a constant variable. In some embodiments, the age prediction model comprises one of the multiple linear regression models described in Table 5.

In some embodiments, the age prediction models further comprise peptide or protein abundances in addition to glycopeptide relative abundances. As such, in some embodiments, the methods provided herein further comprise measuring a concentration of at least one protein in the biological sample and comparing the concentration of the at least one protein to the age prediction model, wherein the age prediction model further comprises the concentration of the at least one protein in the at least one control biological sample. In some embodiments, the at least one protein comprises any of the proteins in Table 2 herein. In some embodiments, the at least one protein comprises IgG3. Protein or peptide concentrations may be measured using any suitable method. In some embodiments, measuring protein or peptide concentration comprises MS (e.g., MRM MS).

### IV. Embodiments

The following embodiments are contemplated. All combinations of features and embodiments are contemplated.

Embodiment 1: A method for determining the age of a biological sample from a subject, the method comprising measuring a relative abundance of at least one glycopeptide in the biological sample and comparing the relative abundance of the at least one glycopeptide to an age prediction model, wherein the age prediction model comprises the relative abundance of the at least one glycopeptide in at least one control biological sample, wherein each control biological sample is from a control individual of a known age, thereby determining the age of the biological sample.

Embodiment 2: An embodiment of embodiment 1, wherein the age of the subject is determined based on the age of the biological sample.

Embodiment 3: An embodiment of embodiment 1 or 2, wherein the at least one glycopeptide comprises any of the glycopeptides in Table 2.

Embodiment 4: An embodiment of any of the embodiments of embodiment 1-3, wherein the at least one glycopeptide comprises IgG1-3510, IgG1-5410, IgM-209-5411, IgM-J-5412, Haptoglobin (Hp)-241-7602, or a combination thereof.

Embodiment 5: An embodiment of any of the embodiments of embodiment 1-4, wherein the at least one glycopeptide comprises IgG1-3510, IgG1-5410, IgM-209-5411, IgM-J-5412, and Haptoglobin (Hp)-241-7602.

Embodiment 6: An embodiment of any of the embodiments of embodiment 1-5, wherein the method further comprises measuring a concentration of at least one protein in the biological sample and comparing the concentration of the at least one protein to the age prediction model, and wherein the age prediction model further comprises the concentration of the at least one protein in the at least one control biological sample.

Embodiment 7: An embodiment of embodiment 6, wherein the at least one protein comprises any of the proteins in Table 2.

Embodiment 8: An embodiment of embodiment 6 or 7, wherein the at least one protein comprises IgG3.

Embodiment 9: An embodiment of embodiment 8, wherein the at least one glycopeptide comprises IgG1-3510, IgG1-5410, IgG2-3410, IgM-209-5411, IgM-J-5412, Hp-241-7602, or a combination thereof.

Embodiment 10: An embodiment of embodiment 8 or 9, wherein the at least one glycopeptide comprises IgG1-3510, IgG1-5410, IgG2-3410, IgM-209-5411, IgM-J-5412, and Hp-241-7602.

Embodiment 11: An embodiment of any of the embodiments of embodiment 1-10, wherein the age prediction model comprises the relative abundance of the at least one glycopeptide in a plurality of control biological samples.

Embodiment 12: An embodiment of any of the embodiments of embodiment 1-11, wherein the biological sample and the control biological sample are liquid samples.

Embodiment 13: An embodiment of any of the embodiments of embodiment 1-12, wherein the biological sample and the control biological sample are blood samples, serum samples, plasma samples, or a combination thereof.

Embodiment 14: An embodiment of any of the embodiments of embodiment 1-13, wherein measuring the relative abundance of the at least one glycopeptide comprises mass spectrometry.

Embodiment 15: An embodiment of any of the embodiments of embodiment 1-14, wherein measuring the relative abundance of the at least one glycopeptide comprises multiple reaction monitoring mass spectrometry.

Embodiment 16: An embodiment of embodiment 15, wherein measuring the relative abundance of the at least one glycopeptide comprises calculating the relative response of the at least one glycopeptide as the area under the mass spectrometry curve of the at least one glycopeptide divided by the area under the curve of a non-glycosylated reference peptide from the same protein as the at least one glycopeptide.

Embodiment 17: An embodiment of any of the embodiments of embodiment 1-16, wherein the age prediction model comprises a linear regression model or a multiple linear regression model based on a correlation between the relative abundance of the at least one glycopeptide in the at least one control biological sample and the age of the control individual.

Embodiment 18: An embodiment of embodiment 17, wherein the age prediction model comprises one of the multiple linear regression models of Table 5.

Embodiment 19: An embodiment of any of the embodiments of embodiment 1-18, wherein the subject is male or female.

Embodiment 20: An embodiment of any of the embodiments of embodiment 1-19, wherein the biological sample is from a criminal forensics investigation

Disclosed herein are materials, compositions, and methods that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are various additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### EXAMPLES

The following examples are offered to illustrate, but not to limit the claimed invention.

### Example 1. Site-Specific Map of the Serum Glycome and Intra- and Inter-Protein Glycan Association in Healthy Volunteers

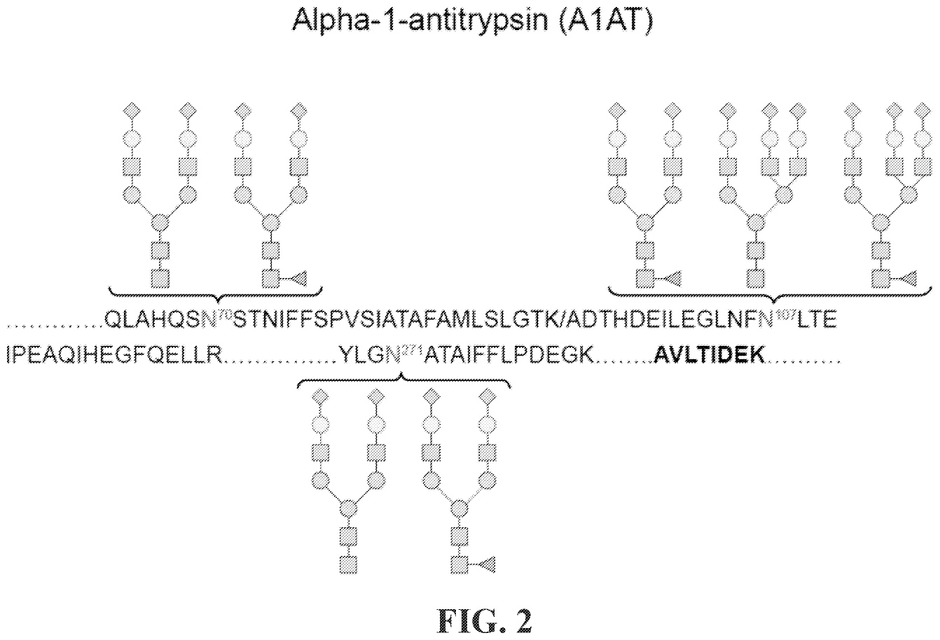

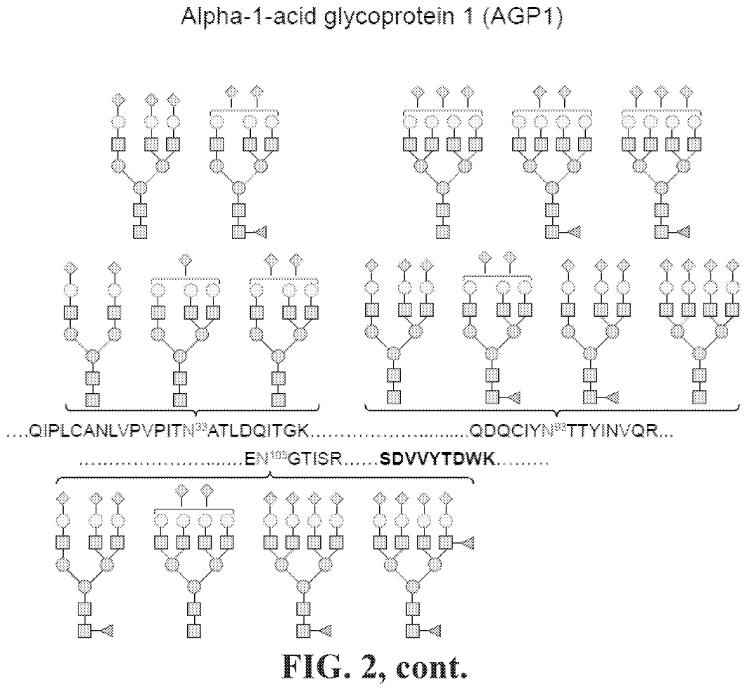

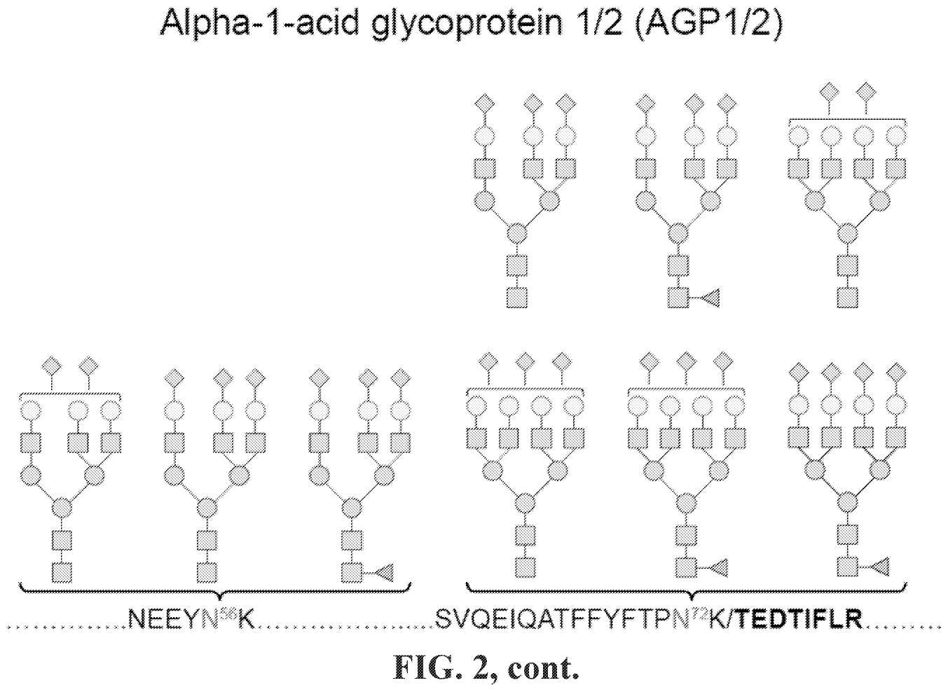

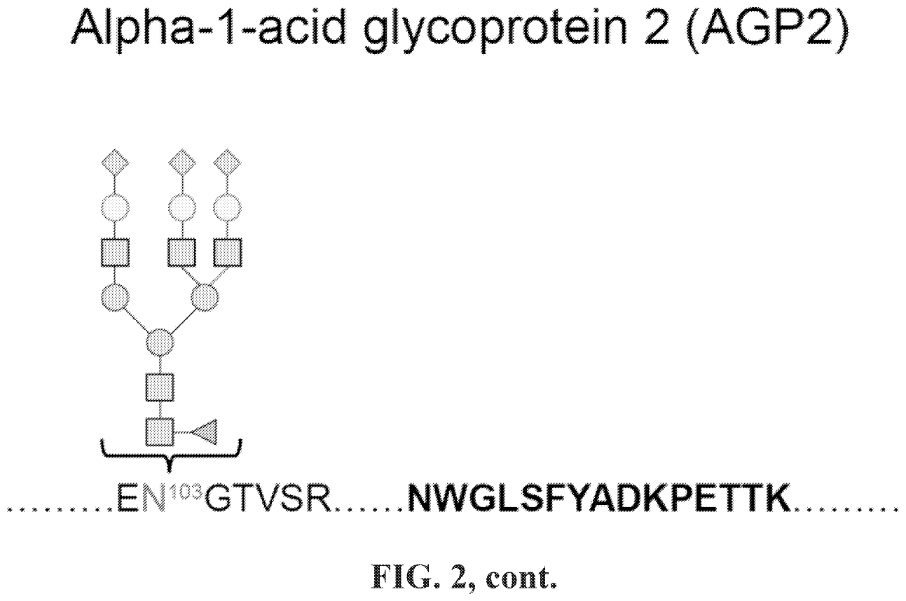

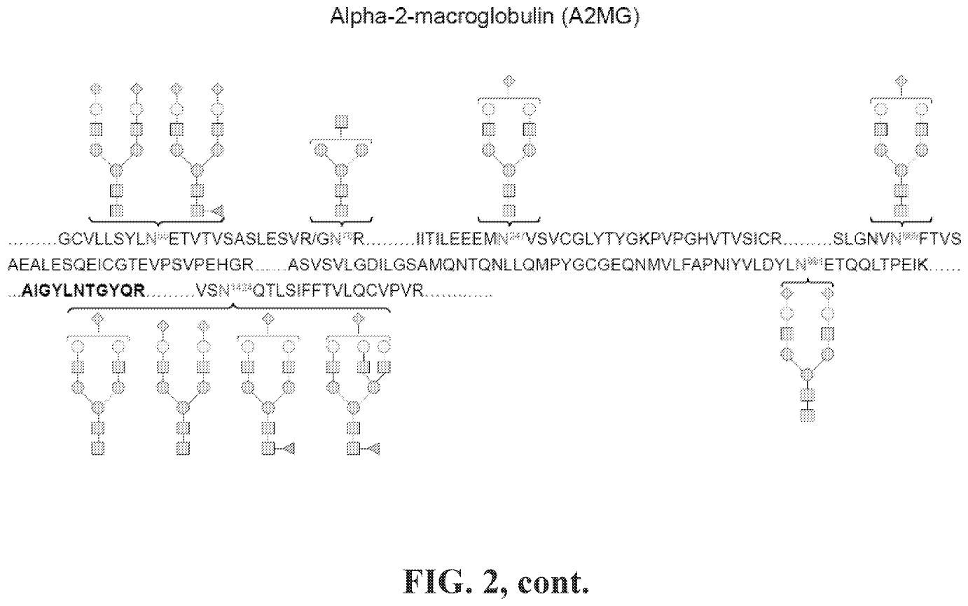

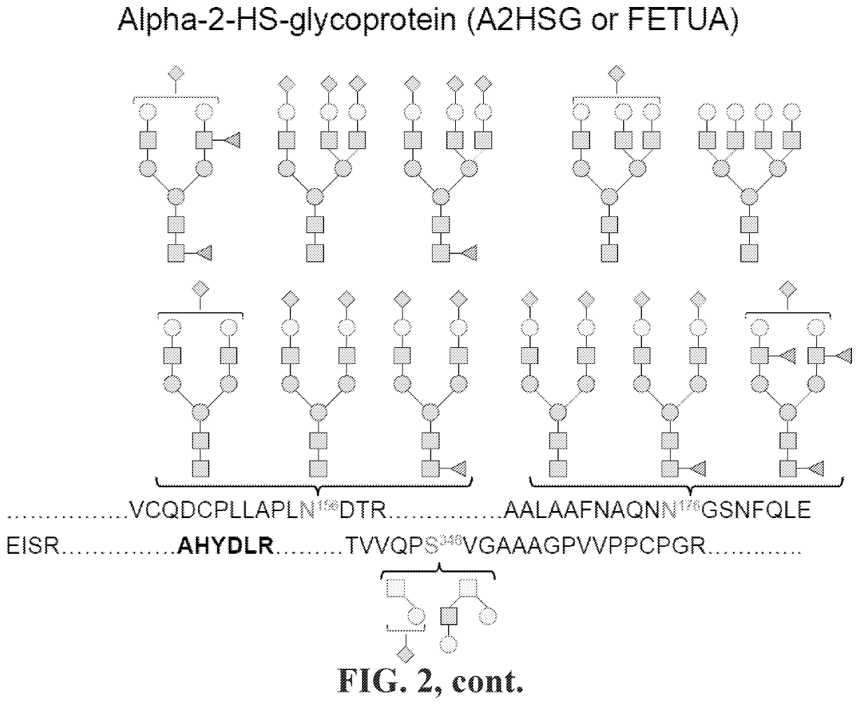

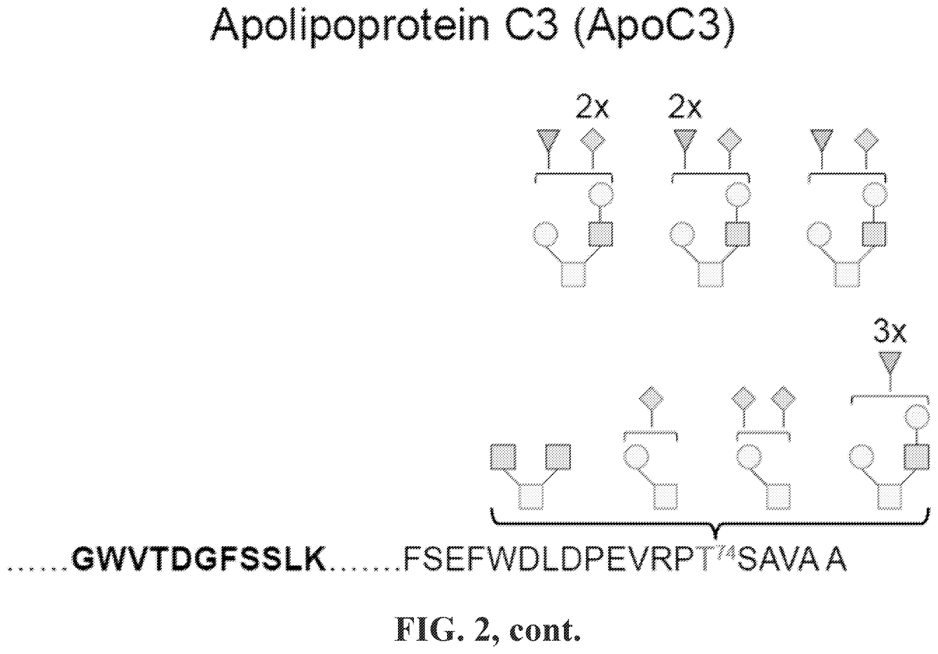

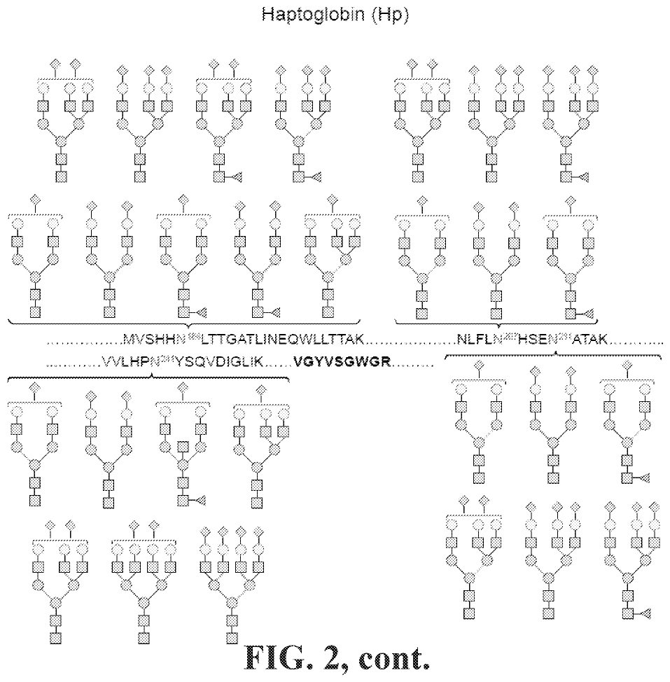

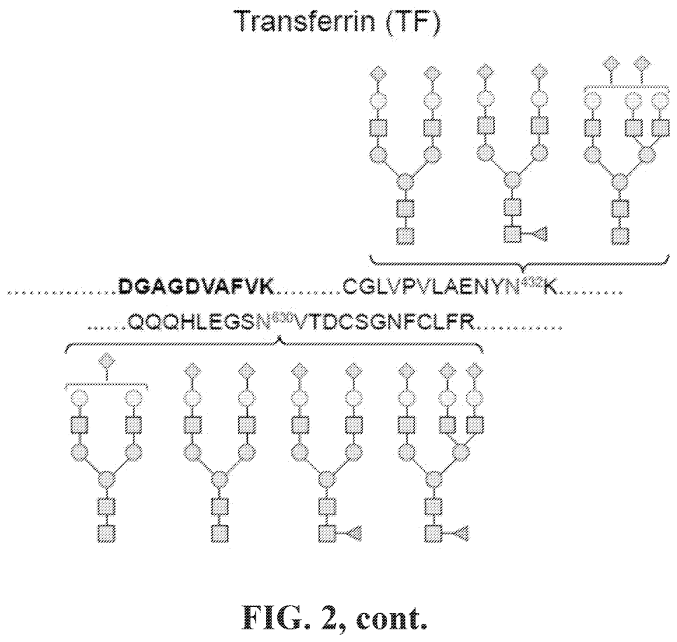

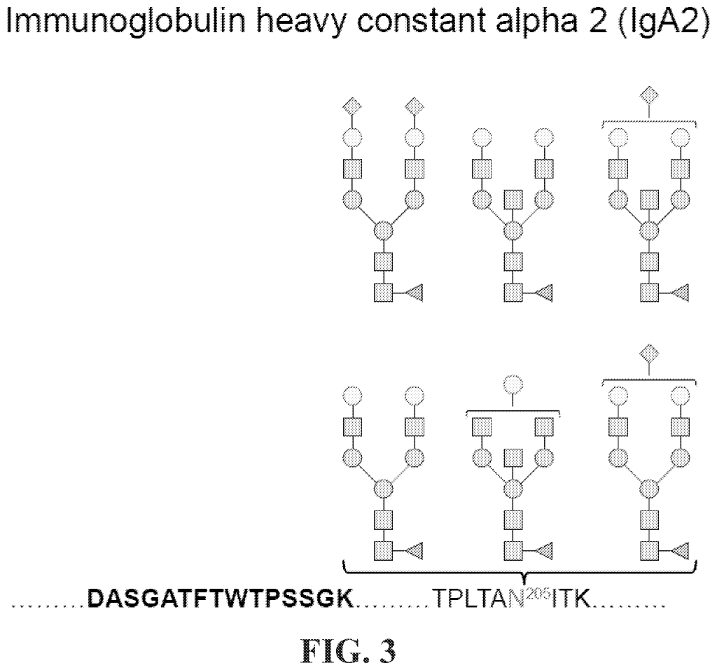

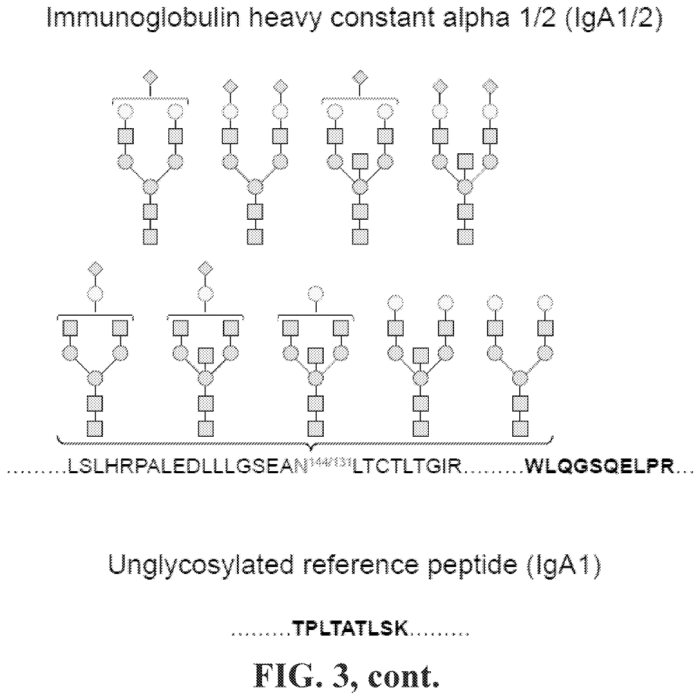

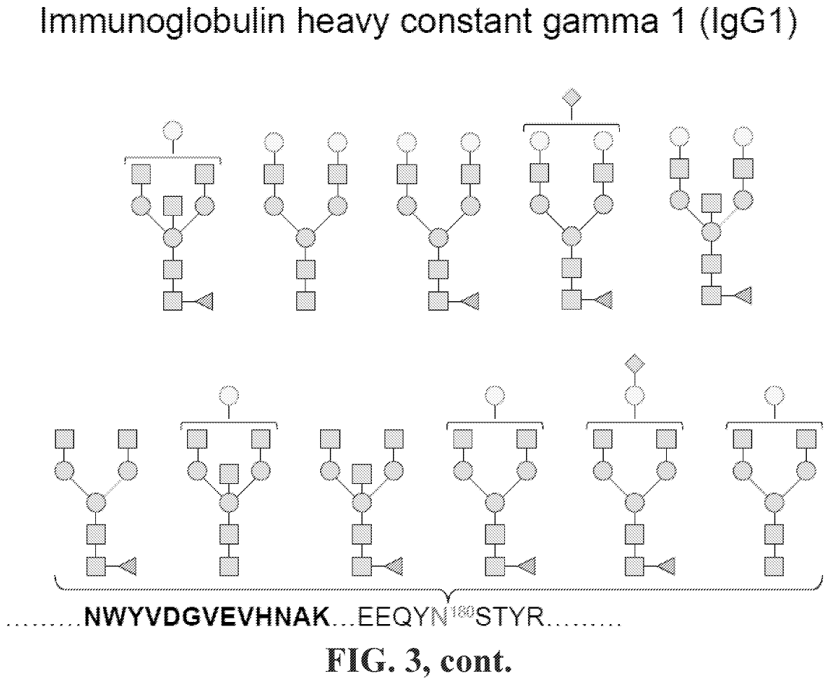

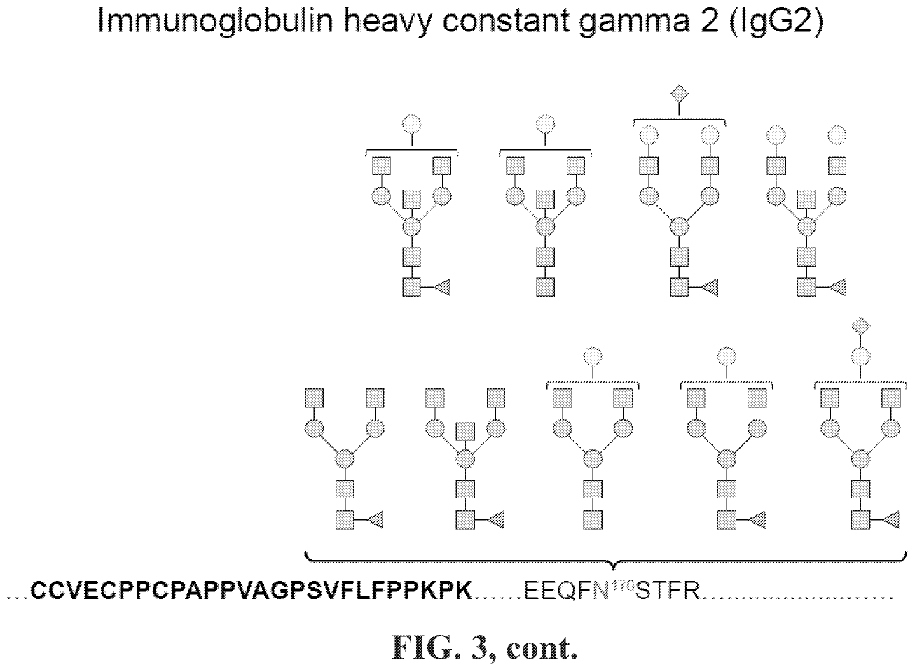

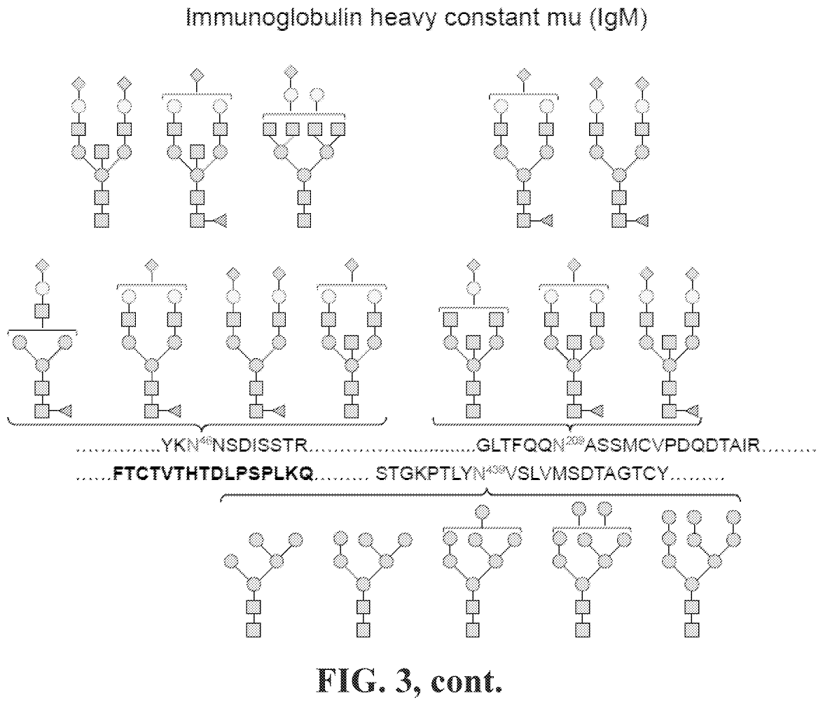

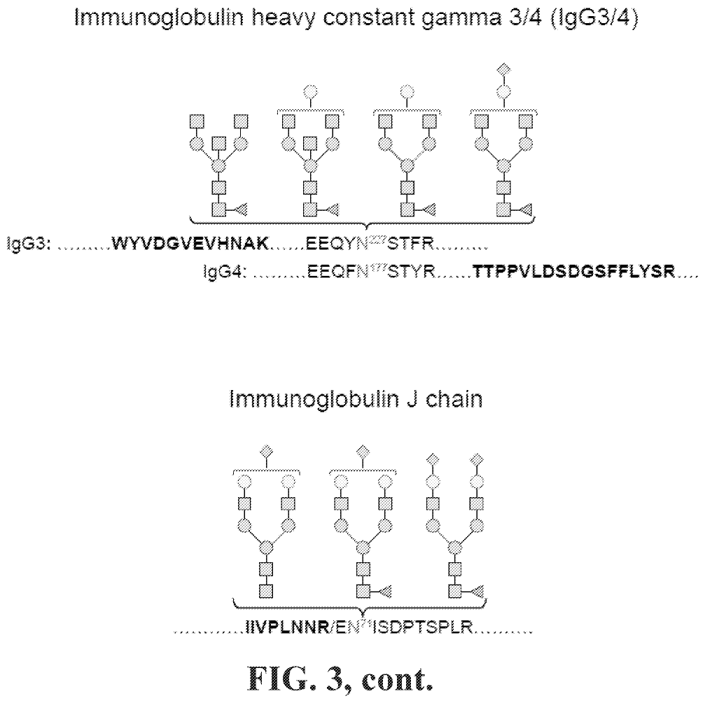

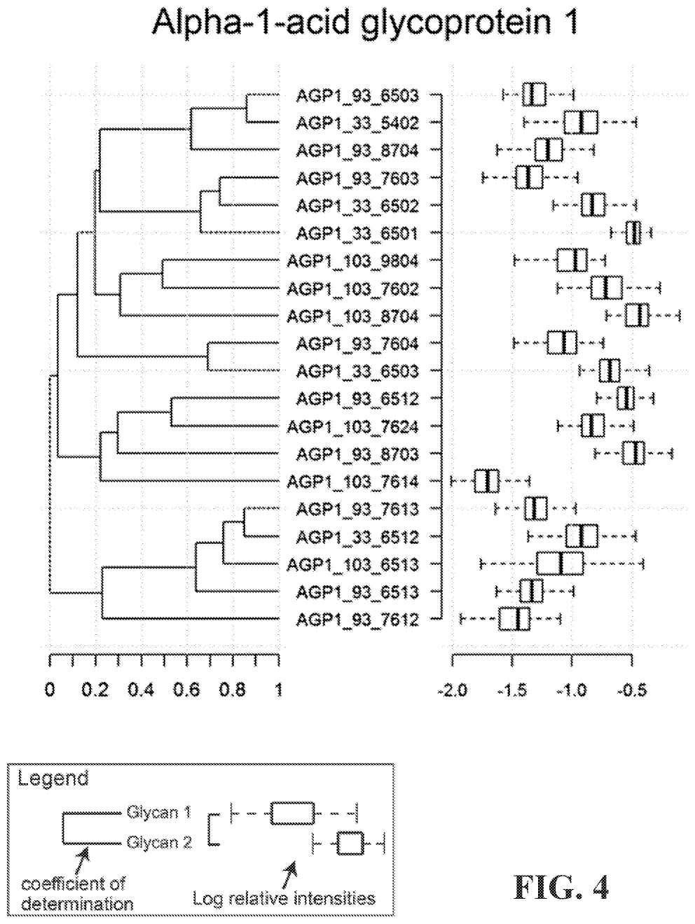

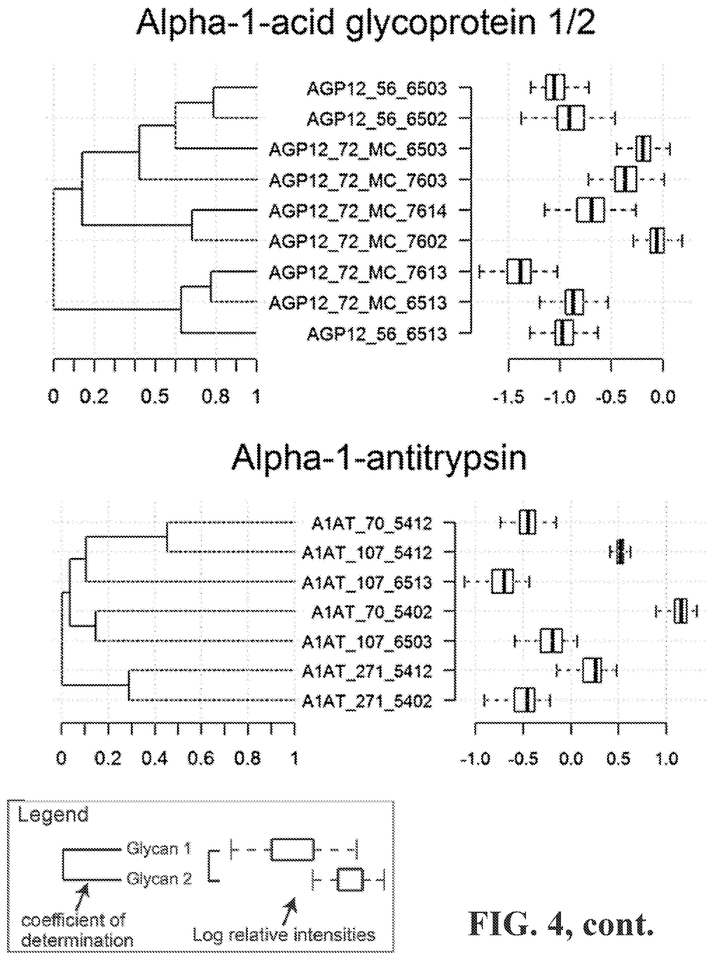

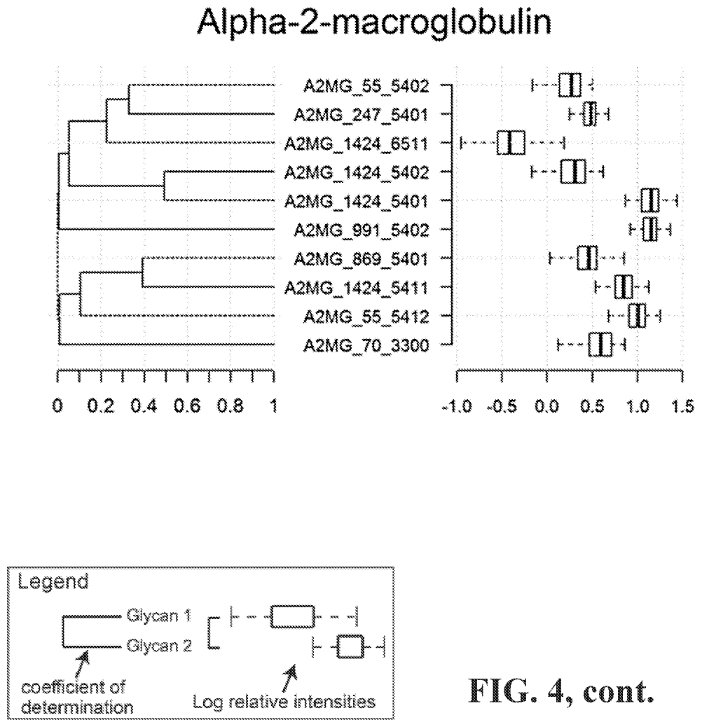

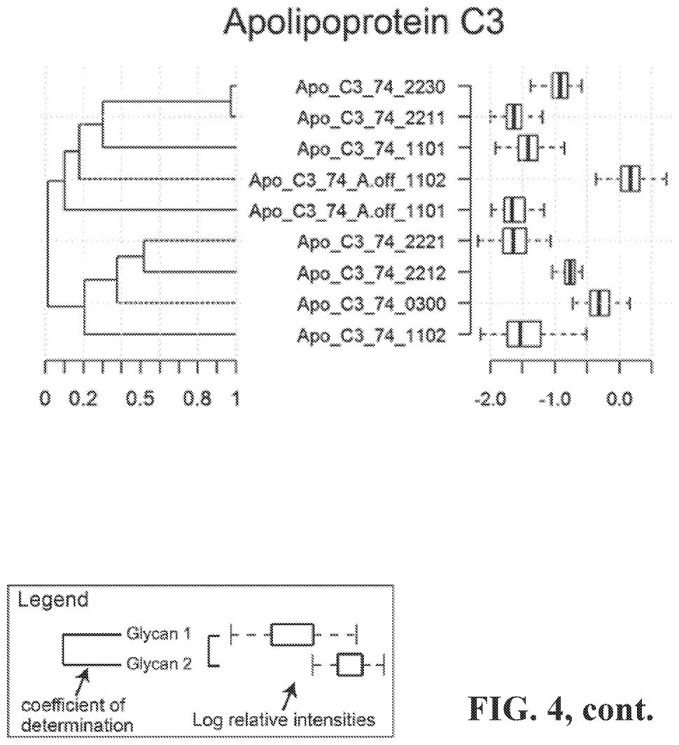

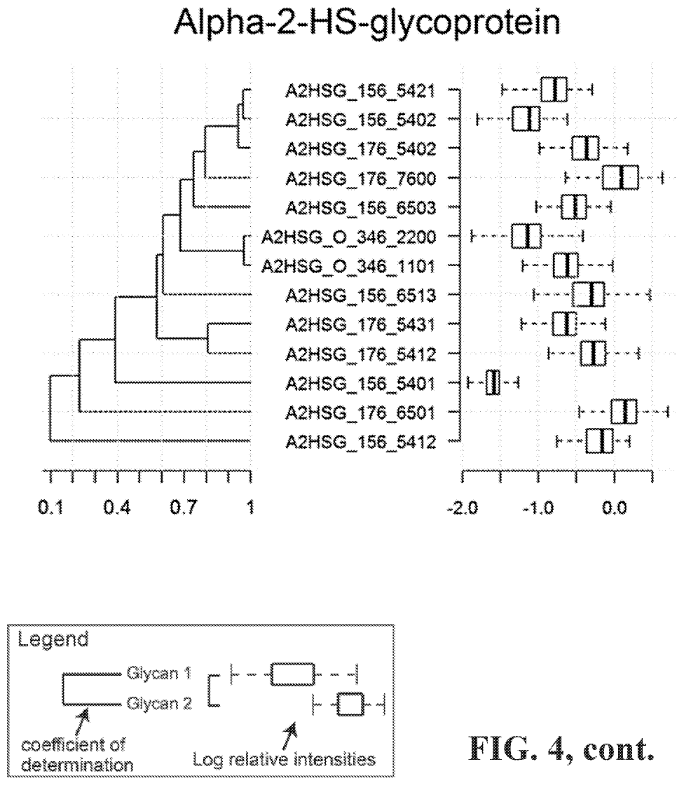

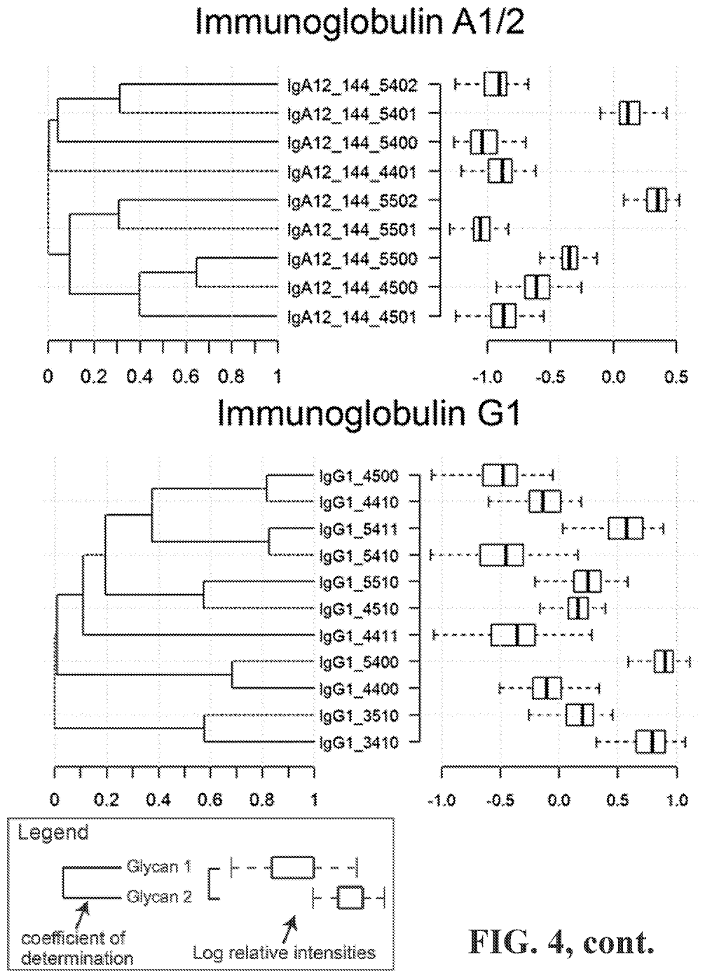

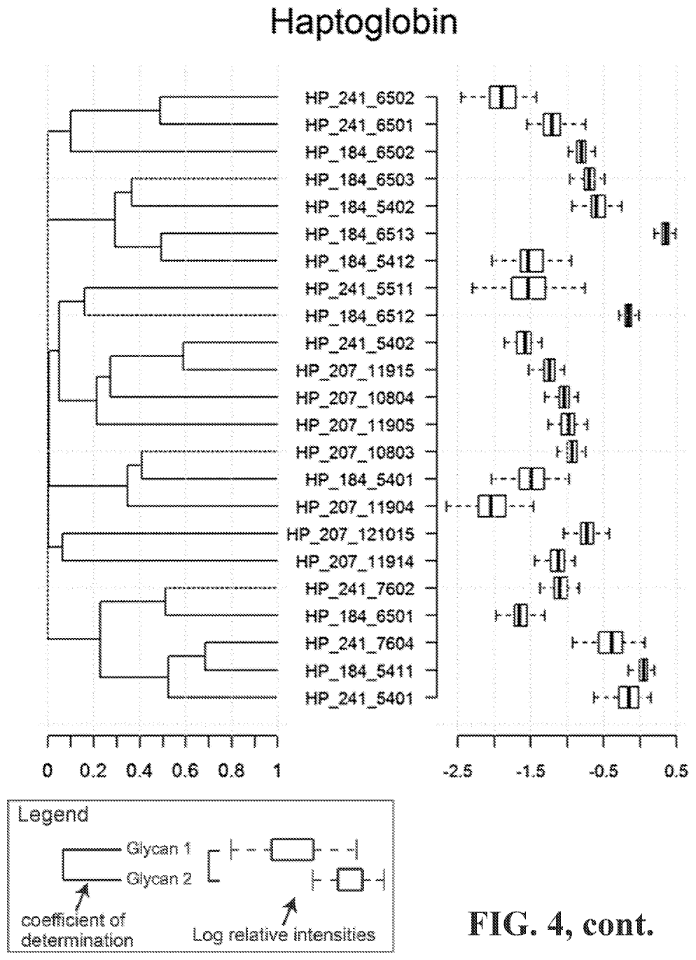

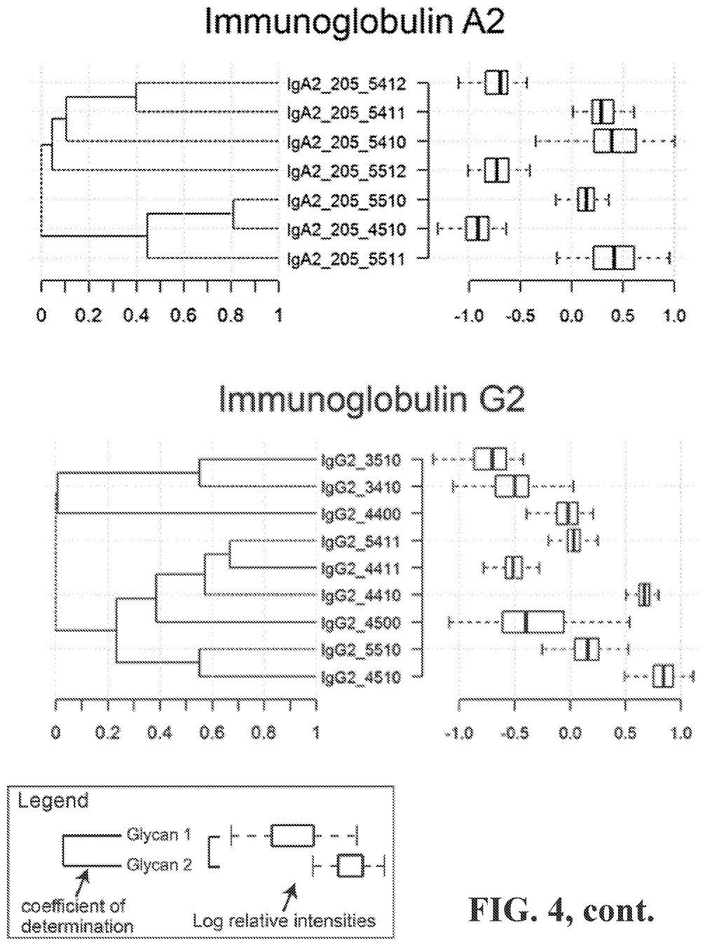

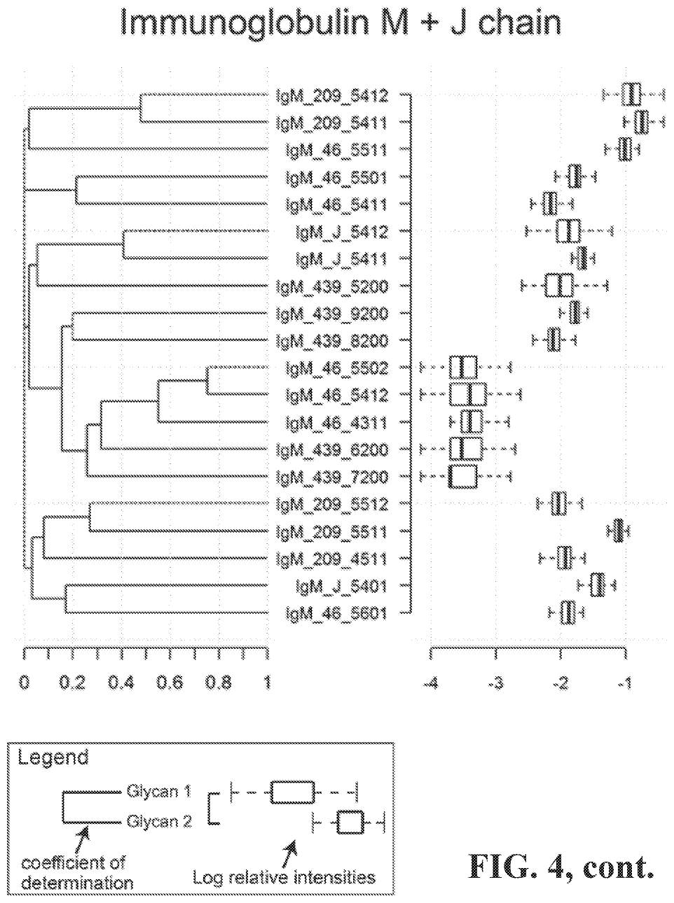

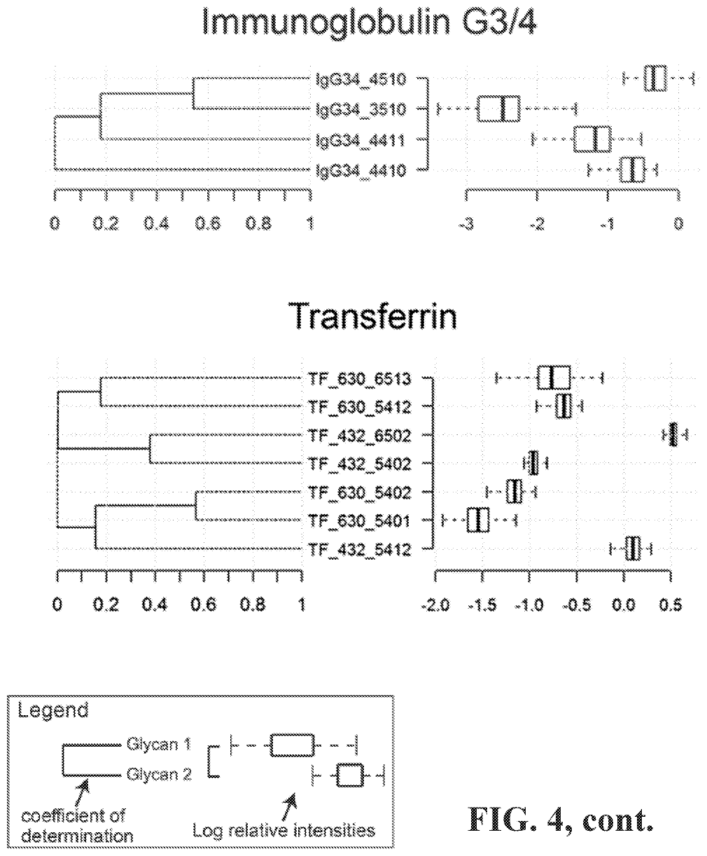

With knowledge of the collision induced dissociation (CID) behavior of the most abundant serum glycoforms (17,23) (FIG. 2 and FIG. 3), the relative abundance of 159 glycopeptides within the serum of 97 healthy volunteers with no known history of thyroid disease, cancer, autoimmunity, or other major medical problem were characterized. For each glycoprotein, a robustly quantified non-glycosylated peptide (FIG. 2 and FIG. 3) was used as an internal reference for calculating each glycoform's relative abundance. Trypsin-digested protein standards were used to calculate each protein's absolute abundance. In total, 159 unique glycopeptides were simultaneously monitored (Table 1) and a site-specific map of the most abundant glycoforms in the human plasma glycome was constructed (FIG. 4).

TABLE 1
Multiple Reaction Monitoring Mass Spectrometry (MRM MS)-monitored transitions

| | | | | Cell | Ret | |
|---|---|---|---|---|---|---|
| | | | CE | Acc | Time | |
| Cpd Name | Ion Monitored | Frag (V) | (V) | (V) | (min) | Polarity |
| A1AT_107_5402 | 1180.57->366.1 | 380 | 30 | 5 | 12.2 | Positive |
| A1AT_107_5411 | 1151.56->366.1 | 380 | 30 | 5 | 16 | Positive |
| A1AT_107_5412 | 1209.78->366.1 | 380 | 30 | 5 | 16.5 | Positive |
| A1AT_107_6503 | 1311.82->366.1 | 380 | 30 | 5 | 17 | Positive |
| A1AT_107_6513 | 1341.03->366.1 | 380 | 30 | 5 | 17 | Positive |
| A1AT_271_5402 | 991.2->366.1 | 380 | 30 | 5 | 11.9 | Positive |
| A1AT_271_5412 | 1027.71->366.1 | 380 | 30 | 5 | 11.9 | Positive |
| A1AT_271_MC_5402 | 1149.93->366.1 | 380 | 30 | 5 | 16 | Positive |
| A1AT_271_MC_5412 | 1179.14->366.1 | 380 | 30 | 5 | 13.8 | Positive |
| A1AT_70_5402 | 1078.49->366.1 | 380 | 30 | 5 | 20.5 | Positive |
| A1AT_70_5412 | 1107.7->366.1 | 380 | 30 | 5 | 20.5 | Positive |
| A2HSG_Peptide | 360.1->519.3 | 380 | 4 | 5 | 0.75 | Positive |
| A2HSG_Peptide | 360.1->289.1 | 380 | 4 | 5 | 0.75 | Positive |
| A2HSG_156_5401 | 1229.18->366.1 | 380 | 20 | 5 | 8.2 | Positive |
| A2HSG_156_5402 | 994.9->366.1 | 380 | 21 | 5 | 8.2 | Positive |
| A2HSG_156_5412 | 1374.89->366.1 | 380 | 22 | 5 | 7.2 | Positive |
| A2HSG_156_5421 | 1326.55->366.1 | 380 | 21 | 5 | 8.2 | Positive |
| A2HSG_156_6502 | 1086.19->366.1 | 380 | 17 | 5 | 8.2 | Positive |
| A2HSG_156_6503 | 1158.97->366.1 | 380 | 18 | 5 | 8.2 | Positive |
| A2HSG_156_6510 | 1234.85->366.1 | 380 | 20 | 5 | 9 | Positive |
| A2HSG_156_6513 | 1195.48->366.1 | 380 | 19 | 5 | 7.1 | Positive |
| A2HSG_176_5401 | 1070.4->366.1 | 380 | 17 | 5 | 9 | Positive |
| A2HSG_176_5402 | 1142.99->366.1 | 380 | 18 | 5 | 9 | Positive |
| A2HSG_176_5412 | 1179.7->366.1 | 380 | 19 | 5 | 9 | Positive |
| A2HSG_176_5431 | 1180.26->366.1 | 380 | 19 | 5 | 9 | Positive |
| A2HSG_176_6501 | 1161.7->366.1 | 380 | 19 | 5 | 10.5 | Positive |
| A2HSG_176_6502 | 1234.27->366.1 | 380 | 20 | 5 | 9.9 | Positive |
| A2HSG_176_6503 | 1307.05->366.1 | 380 | 21 | 5 | 8.2 | Positive |
| A2HSG_176_6512 | 1271.03->366.1 | 380 | 20 | 5 | 9 | Positive |
| A2HSG_176_6513 | 1343.81->366.1 | 380 | 22 | 5 | 9 | Positive |
| A2HSG_176_7600 | 1180.5->366.1 | 380 | 19 | 5 | 9 | Positive |
| A2HSG_O_319_1101 | 913.0865->274.09 | 380 | 25 | 5 | 22.8 | Positive |
| A2HSG_O_319_1111 | 961.779->274.09 | 380 | 25 | 5 | 22.8 | Positive |
| A2HSG_O_346_1101 | 891.44->274.09 | 380 | 25 | 5 | 22.8 | Positive |
| A2HSG_O_346_2110 | 897.11->366.1 | 380 | 25 | 5 | 22.8 | Positive |
| A2HSG_O_346_2200 | 916.12->366.1 | 380 | 25 | 5 | 22.8 | Positive |
| A2HSG_Peptide | 387.69->566.3 | 380 | 5 | 5 | 4 | Positive |
| A2HSG_Peptide | 387.6->288.2 | 380 | 5 | 5 | 4 | Positive |
| A2MG_1424_5401 | 1020.3->366.1 | 380 | 30 | 5 | 17 | Positive |
| A2MG_1424_5402 | 1093.08->366.1 | 380 | 30 | 5 | 17.4 | Positive |
| A2MG_1424_5411 | 1056.82->366.1 | 380 | 30 | 5 | 17 | Positive |
| A2MG_1424_5412 | 1129.59->366.1 | 380 | 30 | 5 | 17 | Positive |
| A2MG_1424_6501 | 1111.59->366.1 | 380 | 30 | 5 | 17 | Positive |
| A2MG_1424_6511 | 1148.1->366.1 | 380 | 30 | 5 | 14.2 | Positive |
| A2MG_247_5200 | 1239.21->1314.16 | 380 | 28 | 5 | 12.9 | Positive |
| A2MG_247_5401 | 1131.02->366.1 | 380 | 30 | 5 | 12.9 | Positive |
| A2MG_247_5402 | 1189.24->366.1 | 380 | 30 | 5 | 12.2 | Positive |
| A2MG_55_5401 | 1078.86->366.1 | 380 | 30 | 5 | 15 | Positive |
| A2MG_55_5402 | 1151.63->366.1 | 380 | 30 | 5 | 16 | Positive |
| A2MG_55_5411 | 1115.37->366.1 | 380 | 30 | 5 | 15 | Positive |
| A2MG_55_5412 | 1188.15->366.1 | 380 | 30 | 5 | 15.5 | Positive |
| A2MG_70_3300 | 721.39->204.1 | 380 | 30 | 5 | 2.2 | Positive |
| A2MG_70_5401 | 1130.53->366.1 | 380 | 30 | 5 | 2.2 | Positive |
| A2MG_70_5402 | 1276.07->366.1 | 380 | 30 | 5 | 2.2 | Positive |
| A2MG_70_5411 | 1203.55->366.1 | 380 | 30 | 5 | 2.2 | Positive |
| A2MG_70_5412 | 1349.1->366.1 | 380 | 30 | 5 | 2.2 | Positive |
| A2MG_70_6511 | 1386.12->366.1 | 380 | 30 | 5 | 2.2 | Positive |
| A2MG_869_5200 | 1158.79->1206.94 | 380 | 27 | 5 | 10 | Positive |
| A2MG_869_5401 | 1066.68->366.1 | 380 | 30 | 5 | 10 | Positive |
| A2MG_869_5402 | 1124.9->366.1 | 380 | 30 | 5 | 10 | Positive |
| A2MG_869_6200 | 1199.3->1206.94 | 380 | 27 | 5 | 10 | Positive |
| A2MG_869_7200 | 1239.82->1206.94 | 380 | 26 | 5 | 10 | Positive |
| A2MG_991_5402 | 1206.28->366.1 | 380 | 30 | 5 | 22.8 | Positive |
| AGP1_103_6503 | 1213.28->366.1 | 380 | 30 | 5 | 2.2 | Positive |
| AGP1_103_6513 | 1261.97->366.1 | 380 | 30 | 5 | 2.2 | Positive |
| AGP1_103_7602 | 1237.96->366.1 | 380 | 30 | 5 | 2.2 | Positive |
| AGP1_103_7603 | 1334.99->366.1 | 380 | 30 | 5 | 2.2 | Positive |
| AGP1_103_7604 | 1074.27->366.1 | 380 | 30 | 5 | 2.2 | Positive |
| AGP1_103_7612 | 1286.64->366.1 | 380 | 30 | 5 | 2.2 | Positive |
| AGP1_103_7613 | 1383.68->366.1 | 380 | 30 | 5 | 2.2 | Positive |
| AGP1_103_7614 | 1110.78->366.1 | 380 | 30 | 5 | 2.2 | Positive |
| AGP1_103_7624 | 1147.3->366.1 | 380 | 30 | 5 | 2.2 | Positive |
| AGP1_103_8703 | 1092.78->366.1 | 380 | 30 | 5 | 2.2 | Positive |
| AGP1_103_8704 | 1165.55->366.1 | 380 | 30 | 5 | 2.2 | Positive |
| AGP1_103_9804 | 1256.84->366.1 | 380 | 30 | 5 | 2.2 | Positive |
| AGP1_33_5402 | 1196.46->366.1 | 380 | 30 | 5 | 7.2 | Positive |
| AGP1_33_6501 | 1214.97->366.1 | 380 | 30 | 5 | 7 | Positive |
| AGP1_33_6502 | 1287.74->366.1 | 380 | 30 | 5 | 7 | Positive |
| AGP1_33_6503 | 1088.61->366.1 | 380 | 30 | 5 | 7.2 | Positive |
| AGP1_33_6512 | 1324.26->366.1 | 380 | 30 | 5 | 7 | Positive |
| AGP1_33_6513 | 1117.83->366.1 | 380 | 30 | 5 | 7.2 | Positive |
| AGP1_33_7603 | 1161.64->366.1 | 380 | 30 | 5 | 6.1 | Positive |
| AGP1_93_6502 | 1122.51->366.1 | 380 | 30 | 5 | 7.2 | Positive |
| AGP1_93_6503 | 1195.28->366.1 | 380 | 30 | 5 | 7.1 | Positive |
| AGP1_93_6512 | 1159.02->366.1 | 380 | 30 | 5 | 8.2 | Positive |
| AGP1_93_6513 | 1231.8->366.1 | 380 | 30 | 5 | 7.1 | Positive |
| AGP1_93_7602 | 1213.79->366.1 | 380 | 30 | 5 | 7.1 | Positive |
| AGP1_93_7603 | 1286.56->366.1 | 380 | 30 | 5 | 7.1 | Positive |
| AGP1_93_7604 | 1087.67->366.1 | 380 | 30 | 5 | 7.2 | Positive |
| AGP1_93_7612 | 1250.3->366.1 | 380 | 30 | 5 | 7 | Positive |
| AGP1_93_7613 | 1323.08->366.1 | 380 | 30 | 5 | 7.1 | Positive |
| AGP1_93_7614 | 1116.88->366.1 | 380 | 30 | 5 | 7.5 | Positive |
| AGP1_93_8703 | 1102.48->366.1 | 380 | 30 | 5 | 7.5 | Positive |
| AGP1_93_8704 | 967.42->366.1 | 380 | 30 | 5 | 7.1 | Positive |
| AGP1_93_8713 | 1131.69->366.1 | 380 | 30 | 5 | 8 | Positive |
| AGP12_56_5402 | 1001.2->366.1 | 380 | 30 | 5 | 1.9 | Positive |
| AGP12_56_6502 | 1122.91->366.1 | 380 | 30 | 5 | 2.1 | Positive |
| AGP12_56_6503 | 1219.94->366.1 | 380 | 30 | 5 | 2.1 | Positive |
| AGP12_56_6513 | 1268.63->366.1 | 380 | 30 | 5 | 2.1 | Positive |
| AGP2_103_6503 | 1208.6->366.1 | 380 | 30 | 5 | 2.1 | Positive |
| AGP2_103_6513 | 1257.29->366.1 | 380 | 30 | 5 | 2.1 | Positive |
| AGP2_103_7603 | 1330.32->366.1 | 380 | 30 | 5 | 2.1 | Positive |
| AGP2_103_7613 | 1379->366.1 | 380 | 30 | 5 | 4 | Positive |
| Apo_C3_74_0300 | 916.09->204.1 | 380 | 14 | 5 | 10 | Positive |
| Apo_C3_74_0310 | 975.44->204.1 | 380 | 15 | 5 | 11.5 | Positive |
| Apo_C3_74_1101 | 931.76->274.09 | 380 | 14 | 5 | 11.9 | Positive |
| Apo_C3_74_1102 | 1028.79->274.09 | 380 | 16 | 5 | 12 | Positive |
| Apo_C3_74_1111 | 980.44->274.1 | 380 | 15 | 5 | 10.5 | Positive |
| Apo_C3_74_1202 | 1096.48->274.1 | 380 | 17 | 5 | 11.5 | Positive |
| Apo_C3_74_1210 | 951.1->366.1 | 380 | 15 | 5 | 22.8 | Positive |
| Apo_C3_74_1300 | 970.1->366.1 | 380 | 15 | 5 | 22.8 | Positive |
| Apo_C3_74_1311 | 837.13->274.1 | 380 | 13 | 5 | 11.5 | Positive |
| Apo_C3_74_2200 | 956.43->366.1 | 380 | 15 | 5 | 22.8 | Positive |
| Apo_C3_74_2211 | 1102.15->274.1 | 380 | 17 | 5 | 17 | Positive |
| Apo_C3_74_2212 | 899.63->274.1 | 380 | 14 | 5 | 13 | Positive |
| Apo_C3_74_2220 | 1053.8->366.1 | 380 | 17 | 5 | 7.8 | Positive |
| Apo_C3_74_2221 | 1150.84->274.1 | 380 | 18 | 5 | 16 | Positive |
| Apo_C3_74_2230 | 1078.8->366.1 | 380 | 17 | 5 | 20.5 | Positive |
| Apo_Peptide 1 | 598.8->854.4 | 380 | 8 | 5 | 8.8 | Positive |
| Apo_Peptide 1 | 598.8->244.1 | 380 | 8 | 5 | 8.8 | Positive |
| Apo_Peptide 2 | 449.71->434.3 | 380 | 6 | 5 | 6 | Positive |
| Apo_Peptide 2 | 449.7->251.1 | 380 | 6 | 5 | 6 | Positive |
| Apo_Peptide 3 | 1069->1097.5 | 380 | 17 | 5 | 11 | Positive |
| Apo_Peptide 3 | 1069->772.4 | 380 | 17 | 5 | 11 | Positive |
| C3_85_5200 | 1158.34->1230.34 | 380 | 33 | 5 | 8 | Positive |
| C3_85_6200 | 909.52->1230.34 | 380 | 22 | 5 | 8 | Positive |
| C3_85_7200 | 950.03->1230.34 | 380 | 22 | 5 | 8 | Positive |
| H2HSG_O_319_1101 | 913.1->274.1 | 380 | 25 | 5 | 10.8 | Positive |
| H2HSG_O_319_1102 | 757.8417->274.1 | 380 | 25 | 5 | 9 | Positive |
| H2HSG_O_319_1111 | 961.779->274.1 | 380 | 25 | 5 | 9 | Positive |
| H2HSG_O_319_1201 | 735.8445->274.1 | 380 | 25 | 5 | 9 | Positive |
| H2HSG_O_346_1102 | 988.4697->274.1 | 380 | 25 | 5 | 16.8 | Positive |
| HP_184_5401 | 1149.4->366.1 | 380 | 30 | 5 | 9.2 | Positive |
| HP_184_5402 | 1222.2->366.1 | 380 | 30 | 5 | 9.9 | Positive |
| HP_184_5411 | 1186->366.1 | 380 | 30 | 5 | 8.5 | Positive |
| HP_184_5412 | 1258.7->366.1 | 380 | 30 | 5 | 9.8 | Positive |
| HP_184_6501 | 992.8->366.1 | 380 | 30 | 5 | 9.2 | Positive |
| HP_184_6502 | 1051->366.1 | 380 | 30 | 5 | 9.8 | Positive |
| HP_184_6503 | 1109.2->366.1 | 380 | 30 | 5 | 10.1 | Positive |
| HP_184_6512 | 1080.2->366.1 | 380 | 30 | 5 | 8.1 | Positive |
| HP_184_6513 | 1138.4->366.1 | 380 | 30 | 5 | 10.1 | Positive |
| HP_184_7602 | 1124->366.1 | 380 | 30 | 5 | 11.3 | Positive |
| HP_207_5401 | 1116.4->366.1 | 380 | 30 | 5 | 4.6 | Positive |
| HP_207_5411 | 1174.6->366.1 | 380 | 30 | 5 | 4.7 | Positive |
| HP_207_5402 | 1247.7->366.1 | 380 | 30 | 5 | 4.7 | Positive |
| HP_207_6502 | 1305.9->366.1 | 380 | 30 | 5 | 4.7 | Positive |
| HP_207_6503 | 1276.9->366.1 | 380 | 30 | 5 | 4.7 | Positive |
| HP_207_6513 | 1335.1->366.1 | 380 | 30 | 5 | 8.8 | Positive |
| HP_241_5401 | 1237.3->366.1 | 380 | 30 | 5 | 6.5 | Positive |
| HP_241_5402 | 1001->366.1 | 380 | 30 | 5 | 8.8 | Positive |
| HP_241_5412 | 1383->366.1 | 380 | 30 | 5 | 8.8 | Positive |
| HP_241_5511 | 1015.5->366.1 | 380 | 30 | 5 | 8.8 | Positive |
| HP_241_6501 | 1019.5->366.1 | 380 | 15 | 5 | 8.3 | Positive |
| HP_241_6502 | 1092.3->366.1 | 380 | 30 | 5 | 8.7 | Positive |
| HP_241_6503 | 1165->366.1 | 380 | 30 | 5 | 9 | Positive |
| HP_241_6512 | 1128.8->366.1 | 380 | 30 | 5 | 8.2 | Positive |
| HP_241_6513 | 1201.5->366.1 | 380 | 30 | 5 | 7.1 | Positive |
| HP_241_7602 | 1183.5->366.1 | 380 | 30 | 5 | 8.4 | Positive |
| HP_241_7603 | 1256.3->366.1 | 380 | 30 | 5 | 11 | Positive |
| HP_241_7604 | 1063.5->366.1 | 380 | 30 | 5 | 8.1 | Positive |
| HP_241_7613 | 1292.8->366.1 | 380 | 30 | 5 | 7.1 | Positive |
| IgA12_144_3500 | 1117.1->366.1 | 380 | 25 | 5 | 13.6 | Positive |
| IgA12_144_4401 | 943.9->366.1 | 380 | 20 | 5 | 14.3 | Positive |
| IgA12_144_4500 | 1157.6->366.1 | 380 | 25 | 5 | 14.2 | Positive |
| IgA12_144_4501 | 1230.4->366.1 | 380 | 30 | 5 | 14.3 | Positive |
| IgA12_144_5400 | 1147.3->366.1 | 380 | 25 | 5 | 14.2 | Positive |
| IgA12_144_5401 | 976.3->366.1 | 380 | 25 | 5 | 14.2 | Positive |
| IgA12_144_5402 | 1292.9->366.1 | 380 | 30 | 5 | 15 | Positive |
| IgA12_144_5500 | 1198.1->366.1 | 380 | 25 | 5 | 13.7 | Positive |
| IgA12_144_5501 | 1016.9->366.1 | 380 | 25 | 5 | 14.2 | Positive |
| IgA12_144_5502 | 1075.1->366.1 | 380 | 25 | 5 | 15.5 | Positive |
| IgA2_205_4510 | 923.5->366.1 | 380 | 25 | 5 | 4.6 | Positive |
| IgA2_205_5410 | 909.8->366.1 | 380 | 18 | 5 | 4.8 | Positive |
| IgA2_205_5411 | 1006.8->366.1 | 380 | 25 | 5 | 4.8 | Positive |
| IgA2_205_5412 | 1103.8->366.1 | 380 | 25 | 5 | 5 | Positive |
| IgA2_205_5510 | 977.5->366.1 | 380 | 19 | 5 | 4.6 | Positive |
| IgA2_205_5511 | 1074.5->366.1 | 380 | 25 | 5 | 4.8 | Positive |
| IgA2_205_5512 | 878.9->366.1 | 380 | 17 | 5 | 5 | Positive |
| IgG1 Peptide | 624.99->1042.55 | 380 | 30 | 5 | 6.6 | Positive |
| IgG1 Peptide | 624.99->521.77 | 380 | 30 | 5 | 6.6 | Positive |

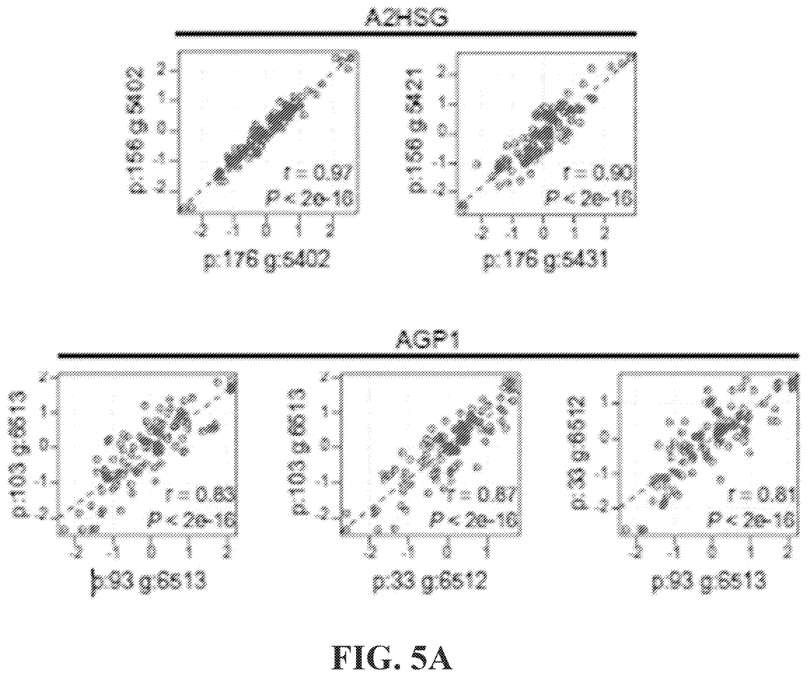

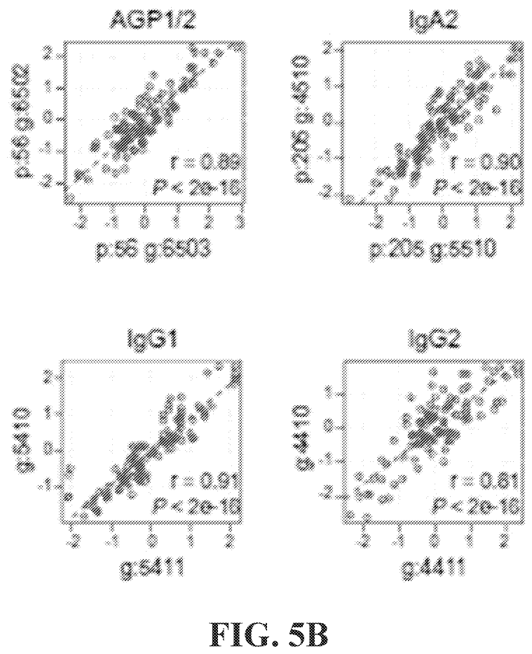

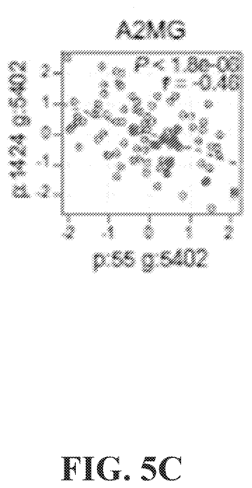

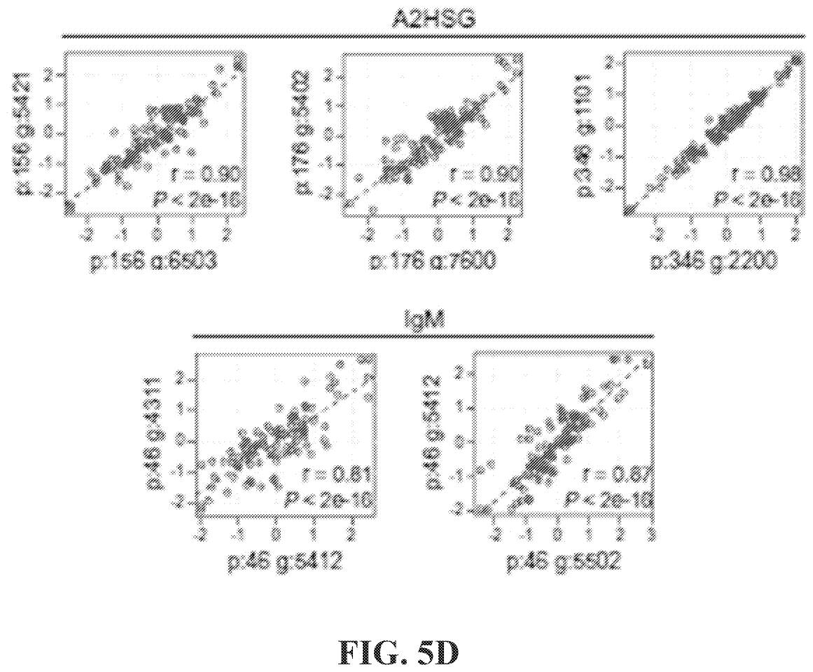

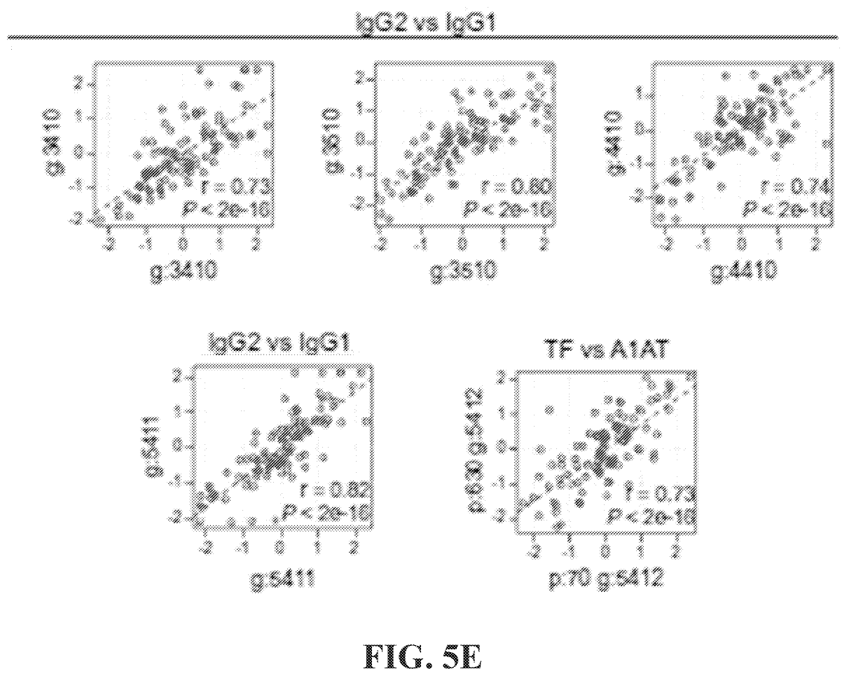

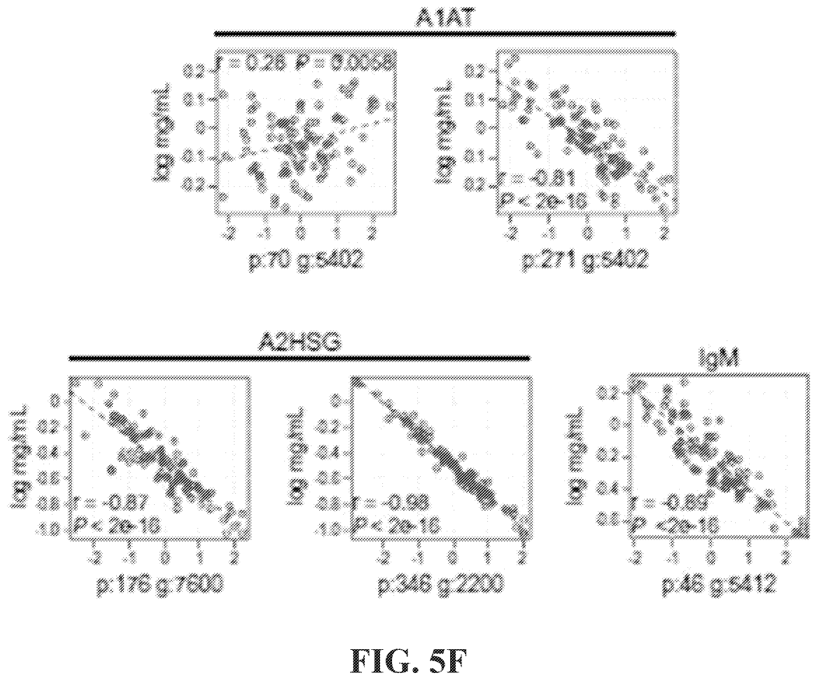

After the relative contribution of each of the glycopeptides that make up the bulk of the plasma glycome was calculated (FIG. 4), their inter-and intra-protein relationships were analyzed (i.e. how the presence of one glycan at a particular site correlates with the expression of other glycans at that site and at distant sites within the same or different glycoprotein). For this analysis, Pearson product-moment correlation coefficients (PPMCCs) were calculated for all possible analyte pairs (FIG. 4 and FIG. 5). This analysis revealed several distinct types of inter-and intra-protein glycan relationships.

Firstly, it was not uncommon for a glycan at one glycosylation site to positively correlate with the same or highly similar glycans at another distant glycosylation site within the same glycoprotein. In other words, structurally similar glycans often occur at different sites within the same protein. For example, the presence of glycan 5402 at position 176 of Alpha-2-HS-glycoprotein (A2HSG) positively correlated (PPMCC 0.974) with the presence of glycan 5402 at site 156 of A2HSG (P<2E-16) (FIG. 5A). Likewise, the presence of glycan 6513 at site 93 of alpha-1-acid glycoprotein (AGP1) positively correlated (PPMCC 0.827) with the presence of glycan 6513 at site 103 of AGP1 (P<2E-16). The previously mentioned glycans (6513 at site 93 and 6513 at site 103) also positively correlated (PPMCC's 0.810 and 0.874, respectively) with a third structurally similar glycan 6512 at site 33 of AGPI (P<2E-16 for both analyte pairs).

In addition to the same or structurally similar glycans tending to occupy different sites within the same protein, glycans of similar structure also tended to occupy the same glycosylation. For example, the presence of glycan 5411 strongly correlated (PPMCC 0.908) with glycan 5410 at the same site of IgG1 (P<2E-16) (FIG. 5B). Thus, the glycosylation machinery of a particular cell can drive the appearance of the same or similar glycans across multiple sites within the same protein.

Although the above examples might seem intuitive, the opposite was also possible, i.e. the relative abundance of a glycan at two different sites within the same glycoprotein can be negatively correlated. For example, glycan 5402 at position 55 of A2MG negatively correlated (PPMCC-0.463) with 5402 at A2MG position 1424 (P=1.84E-06) (FIG. 5C). Thus, in some cases, the cell regulates the presentation of a particular glycan to a specific site, rather than to multiple sites. Finally, there were also examples of structurally distinct glycans residing at the same site positively correlating with one another, an example being glycans 5402 and 7600 which positively correlated (PPMCC 0.900, P<2E-16) with one another at site 176 of alpha 2-HS glycoprotein (A2HSG) (FIG. 5D).

Apart from the intra-protein glycan correlations just described, there were also inter-protein glycan correlations that were of significance, i.e., glycans on different proteins can correlate (positively or negatively) with one another. This was especially true for the different immunoglobulin subclasses. For example, the abundance of glycan modifiers on IgG1 correlated with their identical counterparts on IgG2 (FIG. 4 and FIG. 5E). This is of interest because in theory, IgG1 and IgG2 should be synthesized by different B cell populations, which would indicate that different cells can be influenced to employ similar glycan modifications. Glycan correlations across structurally dissimilar proteins were also sometimes present. One of the most striking of which was the correlation (PPMCC 0.733, P<2E-16) between glycan 5412 at position 70 of Alpha-1 Antitrypsin (A1AT) with glycan 5412 at position 630 of tissue factor (TF) (FIG. 5E). FIG. 6 is a pictorial representation of the 16,742 correlations analyzed in this study. This figure uses t-distributed stochastic neighbor embedding to represent the thousands of correlations as a 2D image, where each symbol represents a different site-specific glycosylation. Symbols that are far away from each other correlate poorly, whereas overlapping symbols are highly correlative. From this image, it is clearly apparent that there are both intra-and inter-glycan correlations. Importantly, previous studies of enzymatically cleaved glycans failed to make such distinctions between populations of glycans originating from different proteins.

Finally, in many cases, the relative abundance of a particular glycan at a defined site correlated with the protein's serum concentration. One interesting example is glycan 5402, which had a small positive correlation (PPMCC 0.28) with A1AT's serum concentration when present at site A1AT site 70 (P=0.006) but had a strong highly significant negative correlation (PPMCC −0.81) with the serum concentration of A1AT when present at A2AT site 271 (P<2E-16) (FIG. 5F). Other examples were the non-sialylated N-glycan 7600 and O-glycan 2200 occurring at sites 176 and 346 of A2HSG, respectively. Both glycans had a strong negative correlation with A2HSG serum concentration (PPMCC-0.87, P<2E-16, and PPMCC-0.98, P<2E-16) (FIG. 5F).

### Example 2. Analysis of Covariates

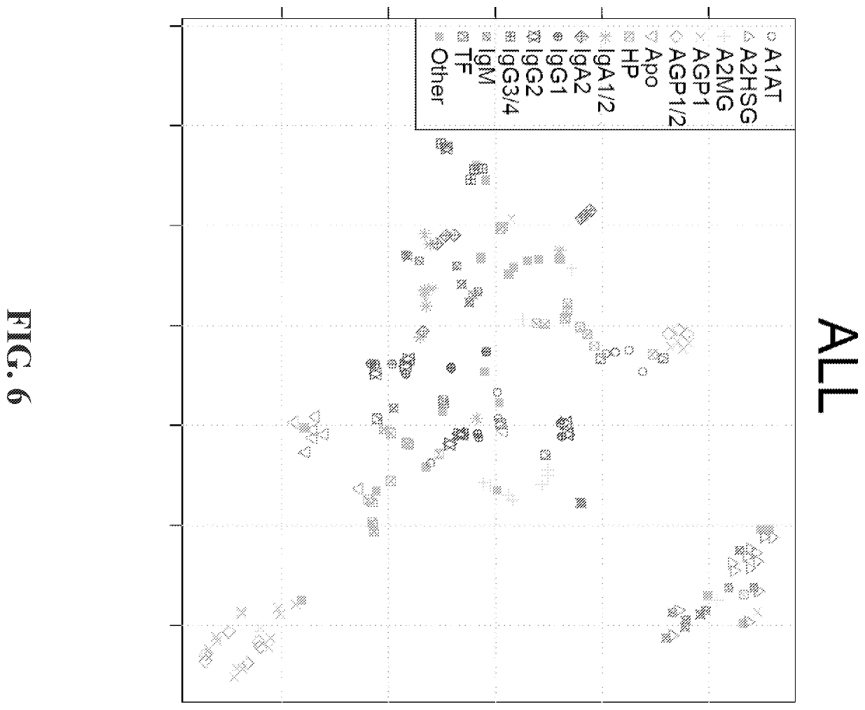

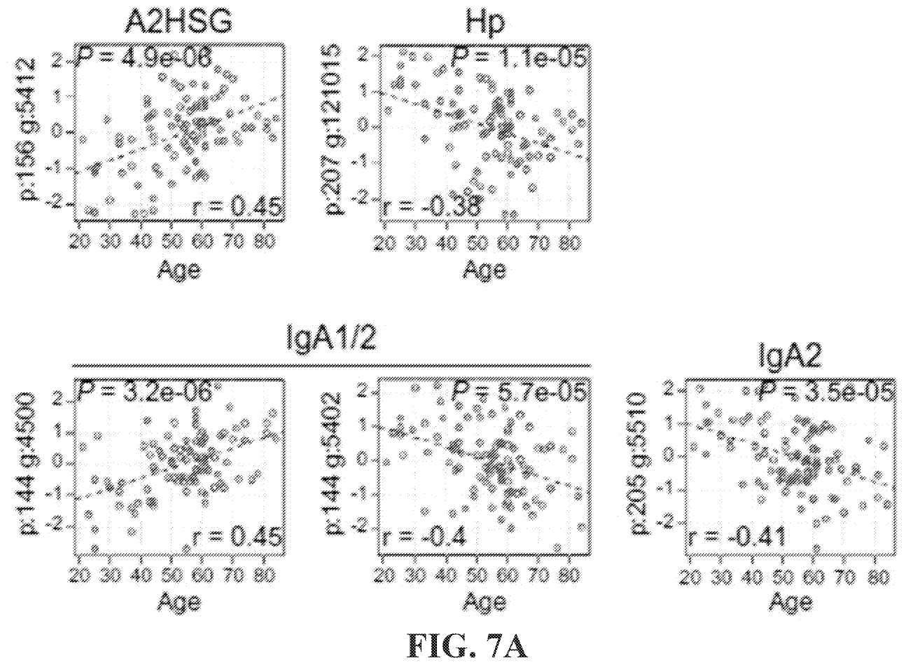

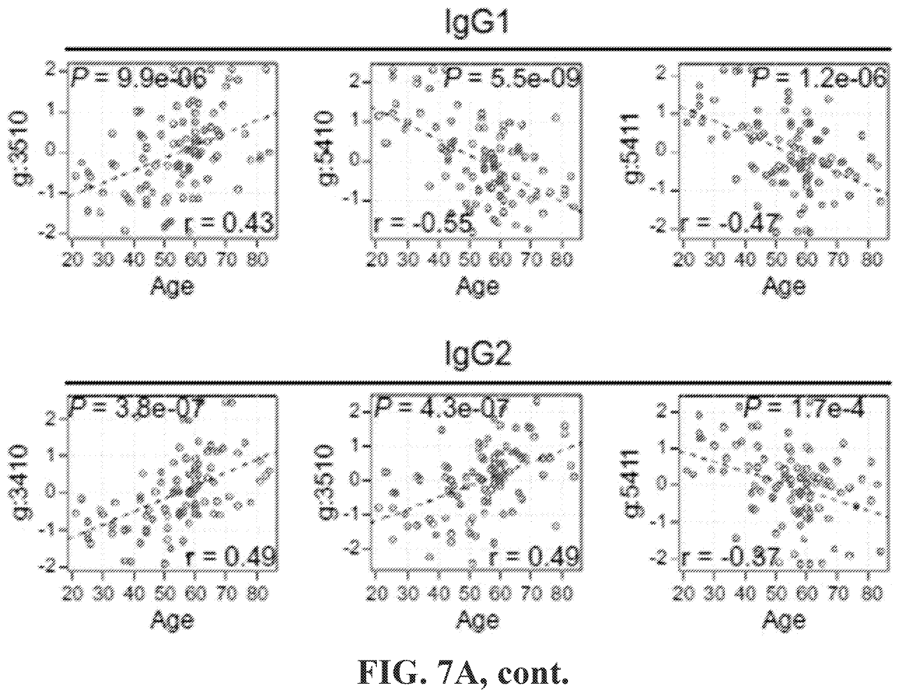

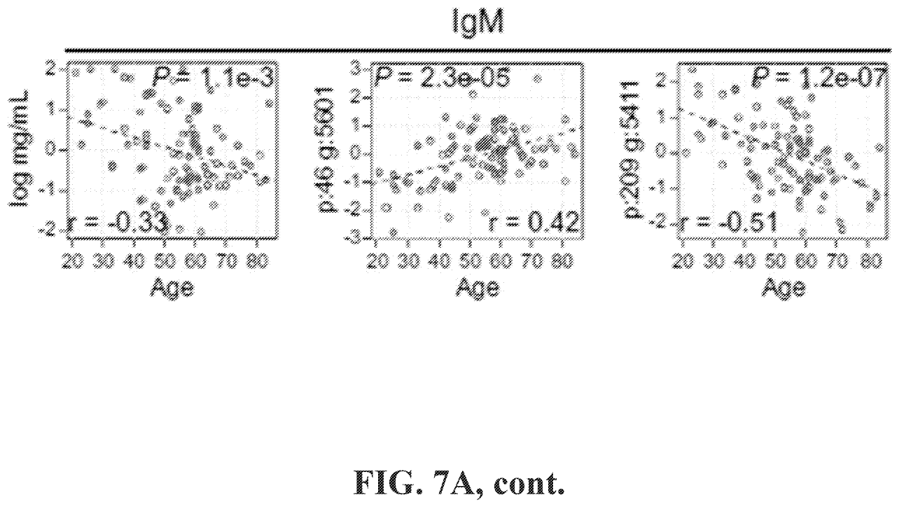

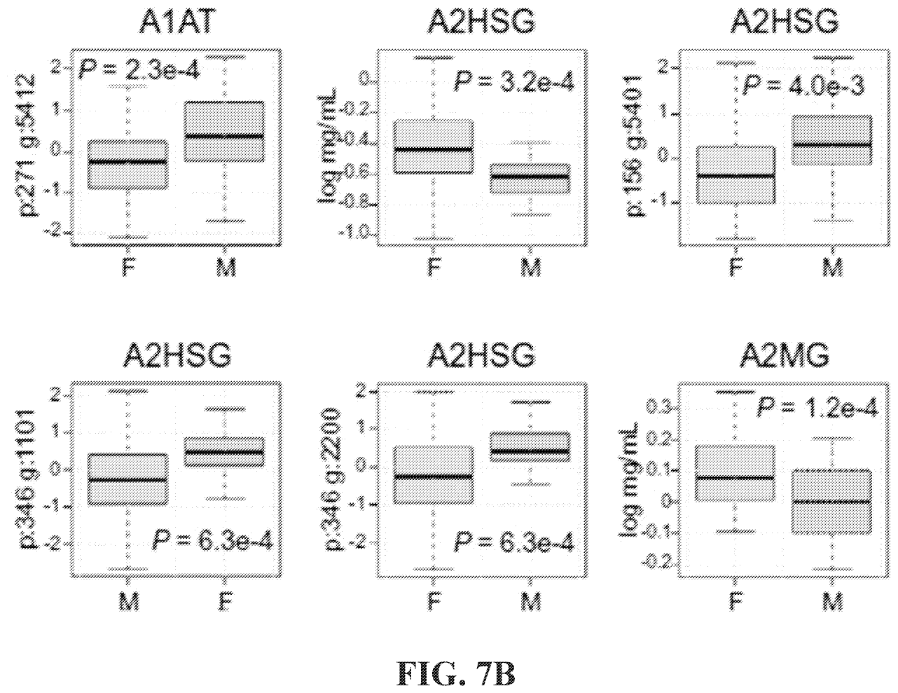

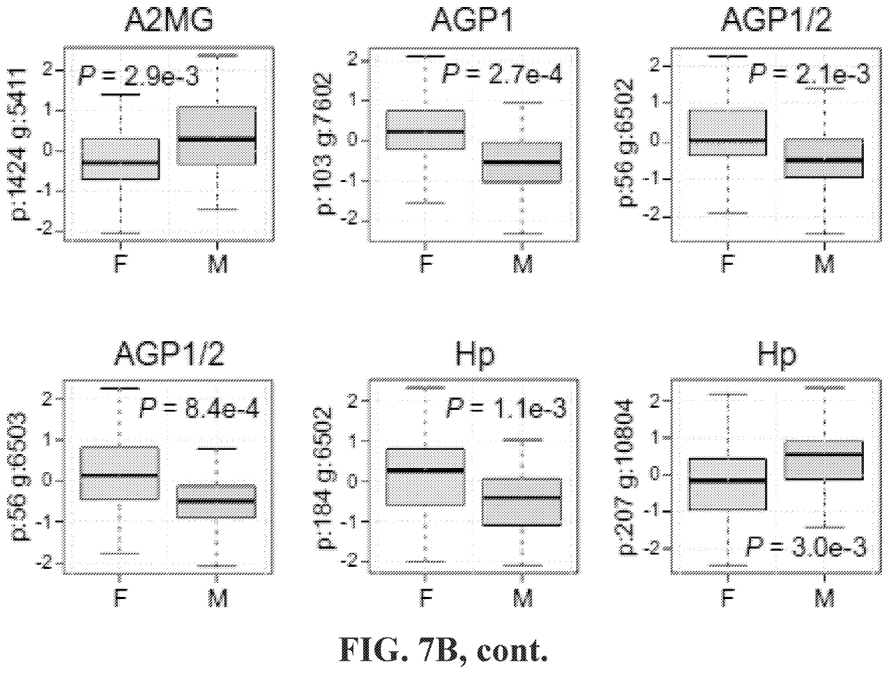

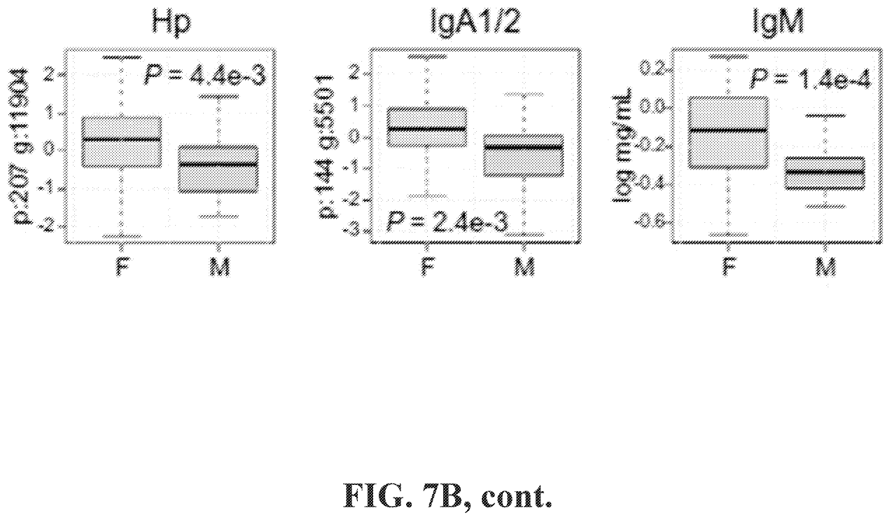

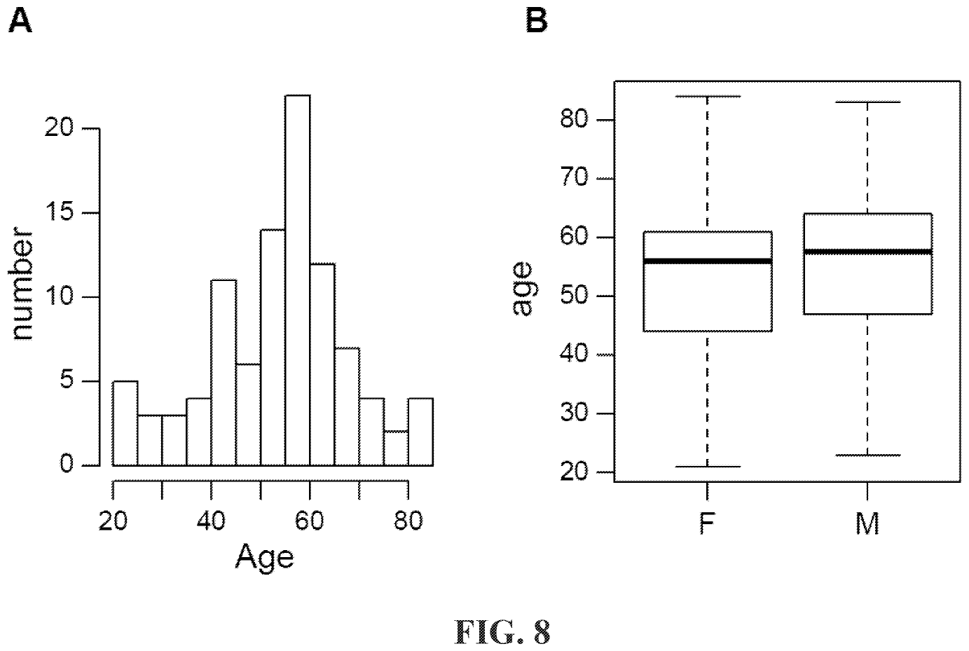

Previous studies conducted mainly on either released glycans or tryptic peptides of purified IgG have demonstrated that age and gender can alter the glycosylation of serum proteins (24-28). Thus, the site-specific glycan alterations that could be contributed to the age and gender effect were characterized (FIG. 7A and FIG. 7B, Table 2, and Table 3). The distribution of age and gender within the healthy control sample set is depicted in FIG. 8A and FIG. 8B. Plotting relative and absolute abundances against age revealed that increasing age is associated with a modest decline in IgM (PPMCC −0.33) (FIG. 7A). The level of IgM was also affected by gender (FDR=0.01), with males showing lower plasma levels of IgM than females (0.49 mg/mL [SD 0.2] vs 0.87 mg/mL [SD 0.6], respectively) (FIG. 7B and Table 4). Of the 159 glycopeptides monitored, the intensities of 41 were associated with age (Table 2).

Importantly, the specific glycan modifications affected by age were consistent across the different IgG subclasses. For example, for IgG1 and IgG2 subclasses, the non-galactosylated 3510 Fc glycan modification was positively correlated with age (PPMCCs 0.43 and 0.49, respectively) (FIG. 7A). In contrast, the fully galactosylated 5411 at this same site was negatively correlated with age (PPMCCs −0.47 and −0.37, respectively). Interestingly, the similar but non-sialylated IgG1 5410 also negatively correlated with age (PPMCC −0.55, P=5.5e-09) (FIG. 7A). Thus, age-glycan relationships depend on more than just the presence or absence of sialylations, which are traditionally thought to be lost during aging.

TABLE 2
Analytes altered by age.

| | ANCOVA | ANCOVA |
|---|---|---|
| Analyte | P value | FDR |
| A2HSG (mg/mL) | 0.00087 | 0.00782 |
| A2HSG p: 156 g: 5402 | 0.01152 | 0.04814 |
| A2HSG p: 156 g: 5412 | 6.1e−06 | 0.00016 |
| A2HSG p: 156 g: 5421 | 0.01190 | 0.04814 |
| A2HSG p: 156 g: 6503 | 0.00544 | 0.02913 |
| A2HSG p: 156 g: 6513 | 0.00746 | 0.03572 |
| A2HSG p: 176 g: 5402 | 0.00389 | 0.02284 |
| A2HSG p: 176 g: 5412 | 0.00659 | 0.03329 |
| A2HSG p: 176 g: 5431 | 0.00971 | 0.04450 |
| A2HSG p: 176 g: 7600 | 0.01186 | 0.04814 |
| A2HSG p: 346 g: 1101 | 0.00046 | 0.00493 |
| A2HSG p: 346 g: 2200 | 0.00074 | 0.00705 |
| ApoC3 p: 74 g: 1102 | 0.01004 | 0.04455 |
| HP p: 207 g: 121015 | 0.00015 | 0.00192 |
| IgA1/2 p: 144 g: 4401 | 0.00717 | 0.03529 |
| IgA1/2 p: 144 g: 4500 | 1.1e−06 | 3.3e−05 |
| IgA1/2 p: 144 g: 4501 | 0.00032 | 0.00390 |
| IgA1/2 p: 144 g: 5401 | 0.00631 | 0.03279 |
| IgA1/2 p: 144 g: 5402 | 5.4e−05 | 0.00089 |
| IgA1/2 p: 144 g: 5500 | 0.00090 | 0.00782 |
| IgA2 p: 205 g: 5412 | 0.00978 | 0.04450 |
| IgA2 p: 205 g: 5510 | 4.1e−05 | 0.00074 |
| IgA2 p: 205 g: 5511 | 0.00106 | 0.00837 |
| IgG1 Norm Resp | 0.00259 | 0.01630 |
| IgG1 g: 3410 | 0.00036 | 0.00405 |
| IgG1 g: 3510 | 1.4e−05 | 0.00032 |
| IgG1 g: 5400 | 0.00201 | 0.01410 |
| IgG1 g: 5410 | 3.2e−09 | 5.9e−07 |
| IgG1 g: 5411 | 3.8e−07 | 1.6e−05 |
| IgG1 g: 5510 | 0.00513 | 0.02828 |
| IgG2 g: 3410 | 4.5e−07 | 1.6e−05 |
| IgG2 g: 3510 | 3.8e−07 | 1.6e−05 |
| IgG2 g: 5411 | 0.00013 | 0.00181 |
| IgM (mg/mL) | 0.00146 | 0.01105 |
| IgM p: 209 g: 4511 | 0.00186 | 0.01358 |
| IgM p: 209 g: 5411 | 7.9e−08 | 7.2e−06 |
| IgM p: 209 g: 5412 | 0.00404 | 0.02296 |
| IgM p: 46 g: 5412 | 0.00220 | 0.01484 |
| IgM p: 46 g: 5502 | 0.01053 | 0.04562 |
| IgM p: 46 g: 5601 | 3.1e−05 | 0.00062 |
| IgM J g: 5401 | 0.00260 | 0.01630 |
| IgM J g: 5412 | 0.00050 | 0.00503 |
| Relative IgM | 0.00286 | 0.01732 |
| IgM p: 439 Ungly | 0.00010 | 0.00156 |
| TF p: 630 g: 6513 | 0.00098 | 0.00810 |
| IgG2 g: 5400 | 0.00201 | 0.01410 |
| IgG2 g: 5410 | 3.2e−09 | 5.9e−07 |
| IgG2 g: 5510 | 0.00513 | 0.02828 |

FDR: false discovery rate; ANCOVA: analysis of covariance.

TABLE 3
Analytes altered by gender.

| | ANCOVA | ANCOVA |
|---|---|---|
| Analyte | P value | FDR |
| A1AT p: 271 g: 5412 | 0.00023 | 0.012 |
| A2HSG (mg/mL) | 0.00032 | 0.012 |
| A2HSG p: 156 g: 5401 | 0.00400 | 0.045 |
| A2HSG p: 346 g: 1101 | 0.00063 | 0.016 |
| A2HSG p: 346 g: 2200 | 0.00063 | 0.016 |
| A2MG (mg/mL) | 0.00012 | 0.012 |
| A2MG p: 1424 g: 5411 | 0.00293 | 0.039 |
| AGP1 p: 103 g: 7602 | 0.00027 | 0.012 |
| AGP12 p: 56 g: 6502 | 0.00212 | 0.039 |
| AGP12 p: 56 g: 6503 | 0.00084 | 0.019 |
| Hp p: 184 g: 6502 | 0.00105 | 0.021 |
| Hp p: 207 g: 10804 | 0.00298 | 0.039 |
| Hp p: 207 g: 11904 | 0.00435 | 0.047 |
| IgA12 p: 144 g: 5501 | 0.00239 | 0.039 |
| IgM (mg/mL) | 0.00014 | 0.012 |
| Relative IgM | 0.00342 | 0.041 |
| IgM p: 439 Ungly | 0.00285 | 0.039 |

FDR: false discovery rate; ANCOVA: analysis of covariance.

TABLE 4
Proteins altered by gender.

| Analyte conc. | | | | |
|---|---|---|---|---|
| (mg/mL) | Female | Male | P value | FDR |
| A1AT | 0.96 ± 0.3 | 0.81 ± 0.2 | 0.00522 | 0.053 |
| A2HSG | 0.44 ± 0.3 | 0.25 ± 0.1 | 0.00032 | 0.012 |
| A2MG | 1.3 ± 0.4 | 1 ± 0.3 | 0.00012 | 0.012 |
| IgM | 0.87 ± 0.6 | 0.49 ± 0.2 | 0.00014 | 0.012 |

FDR: false discovery rate.

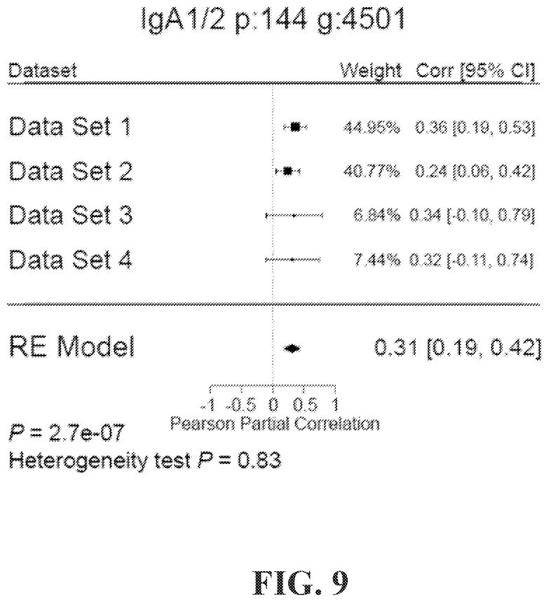

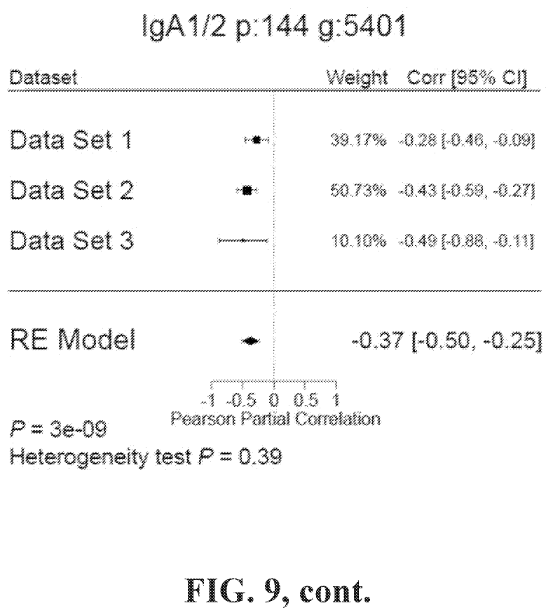

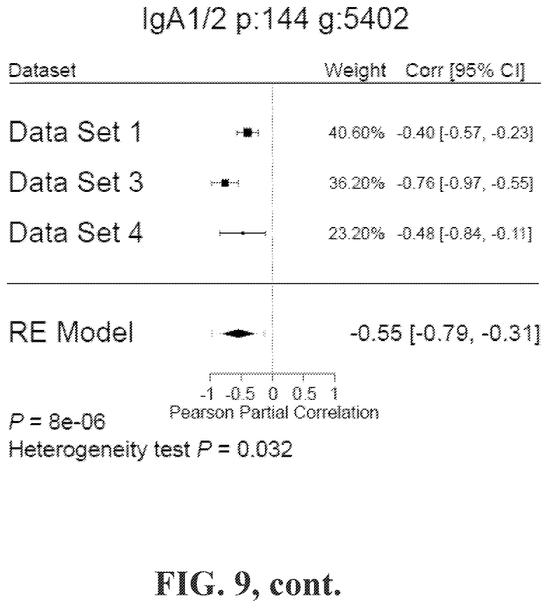

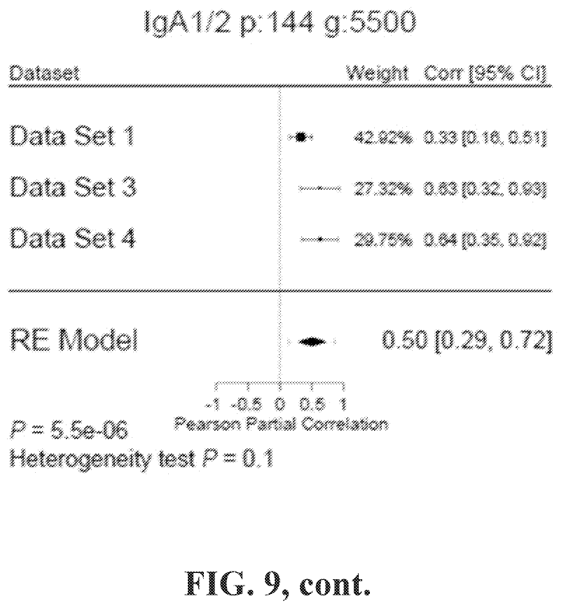

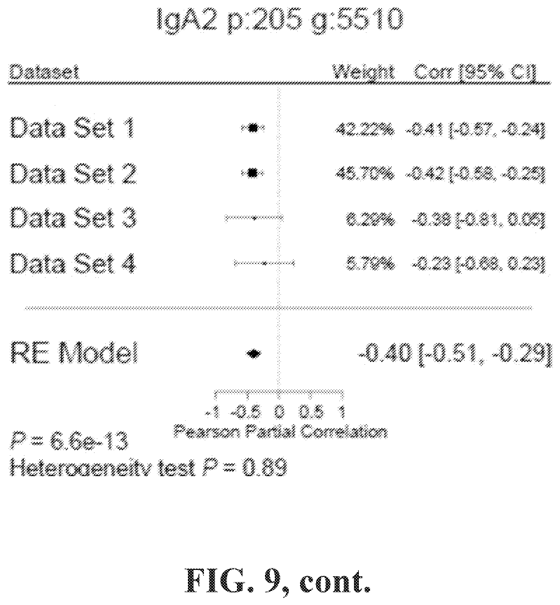

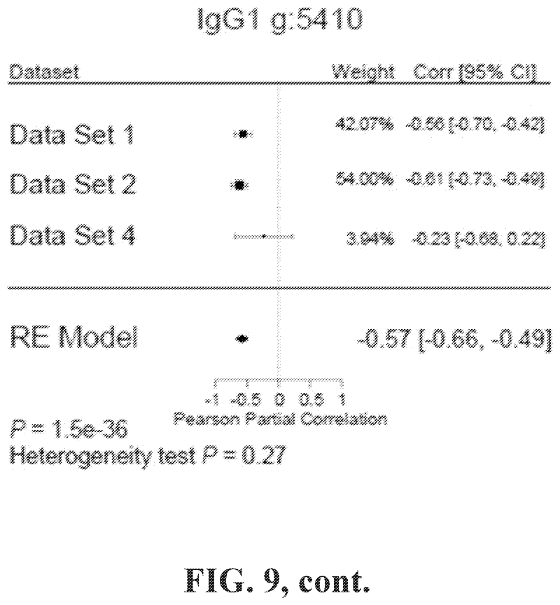

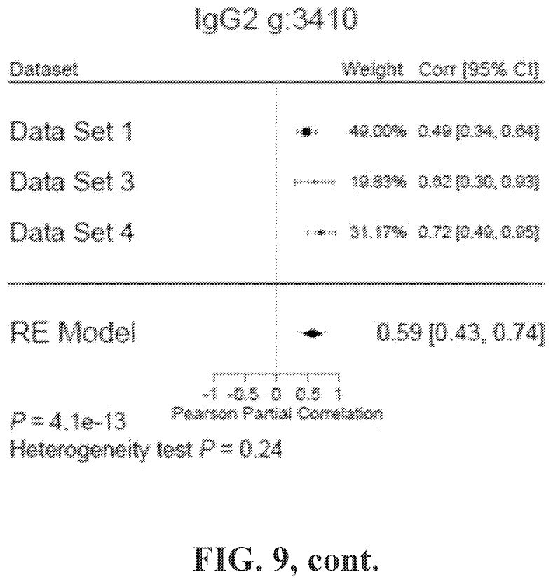

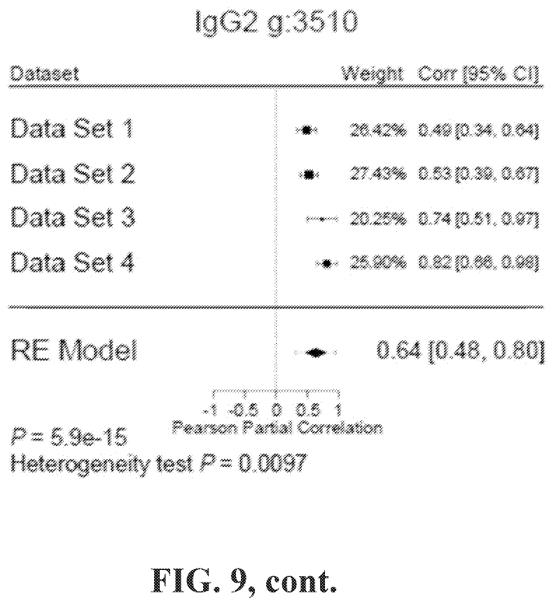

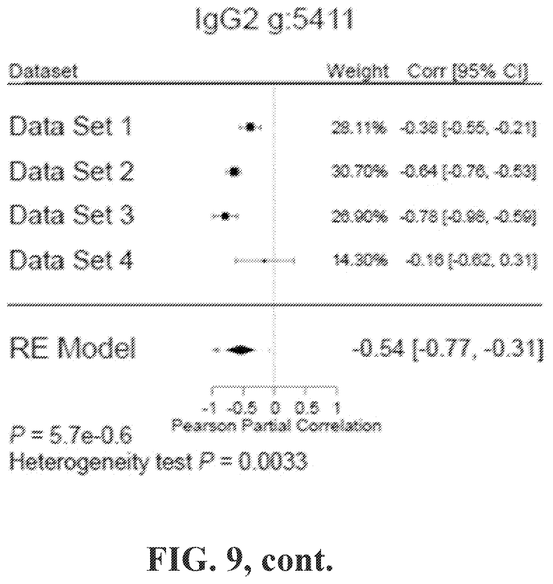

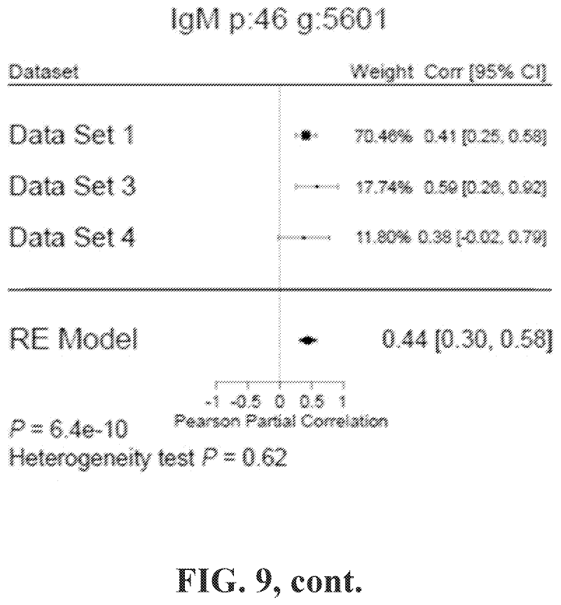

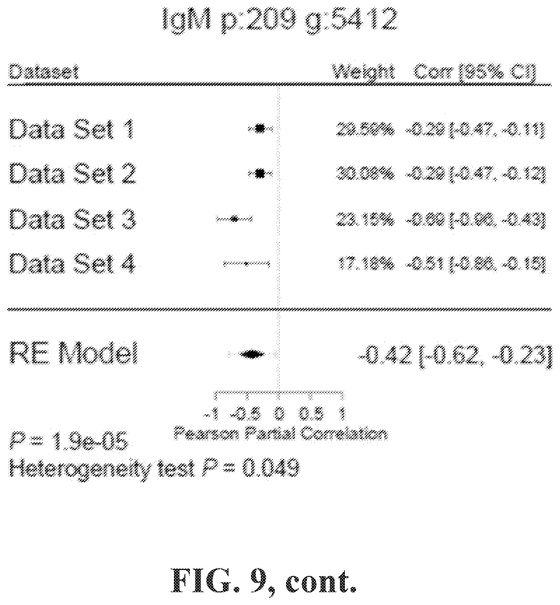

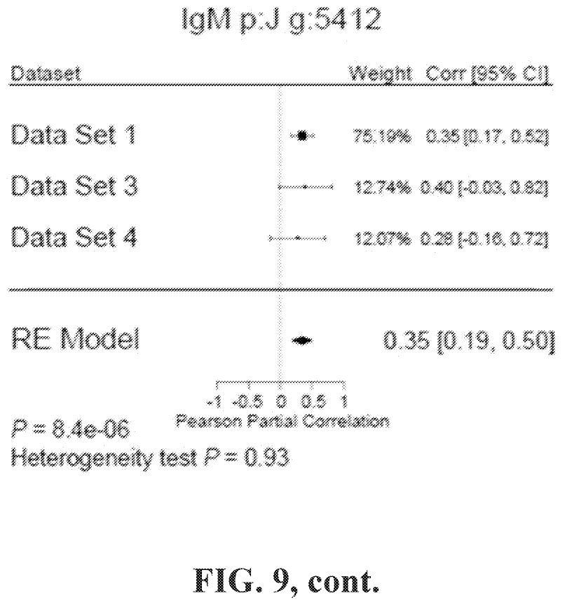

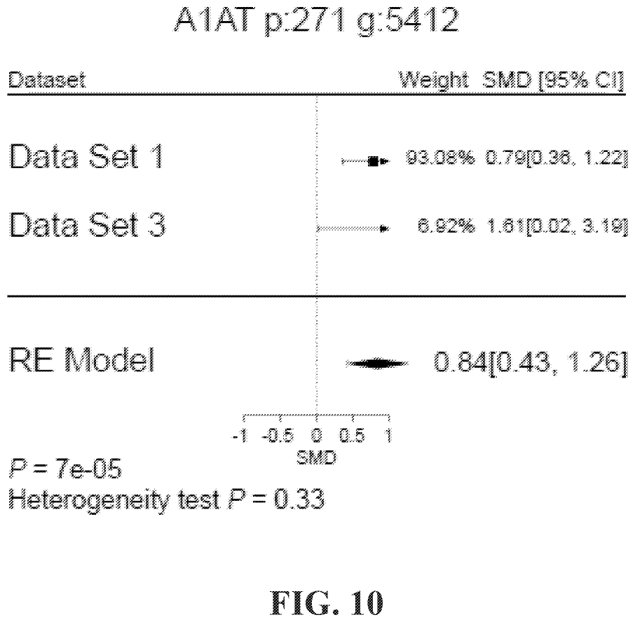

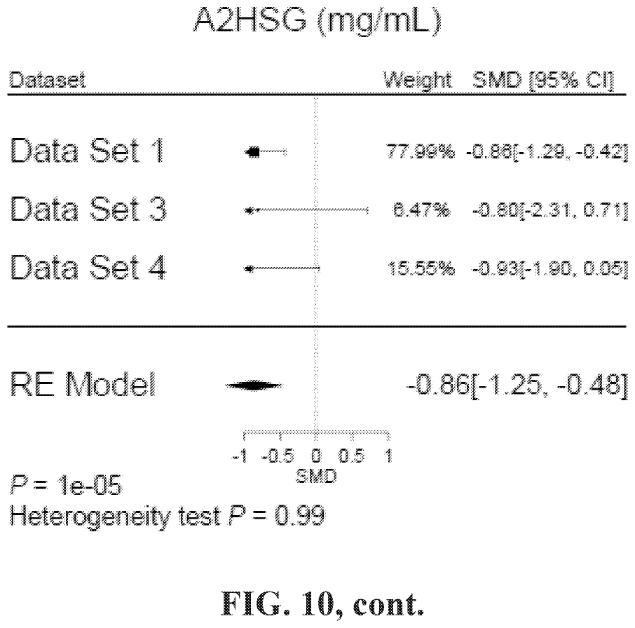

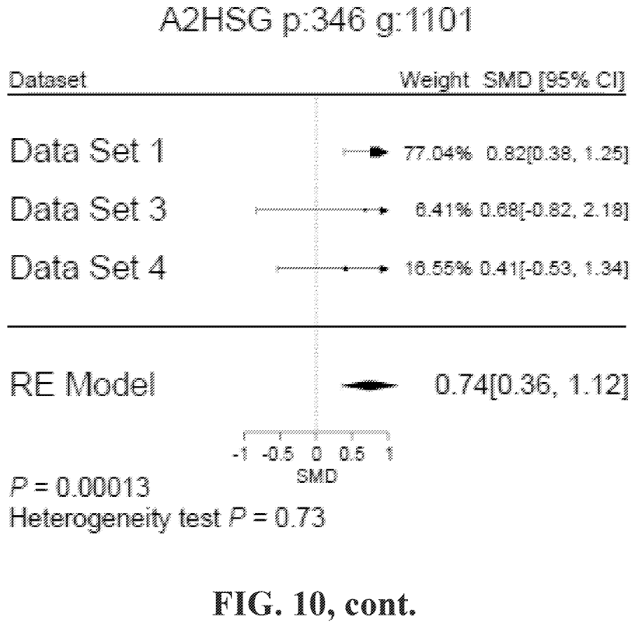

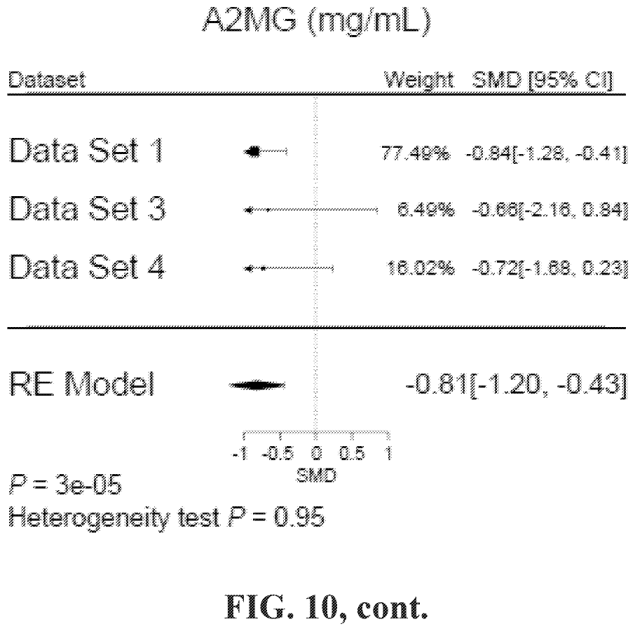

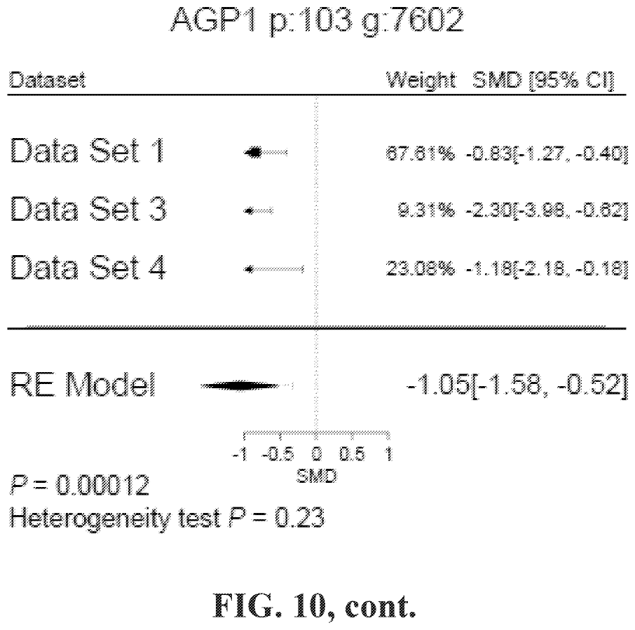

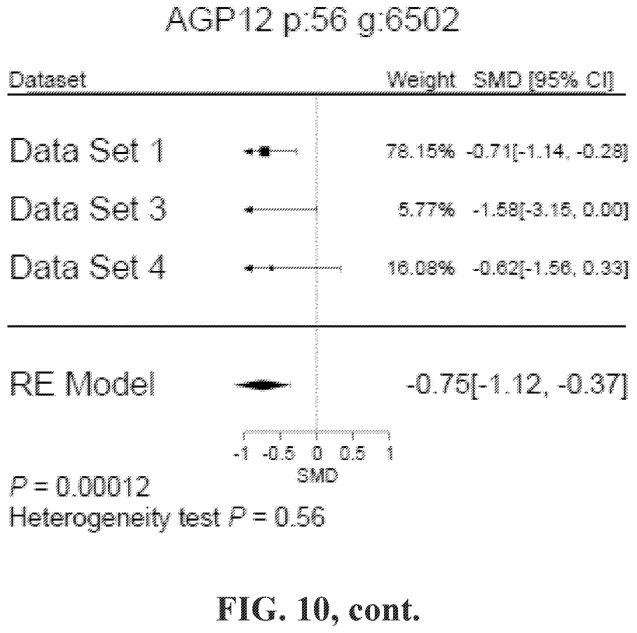

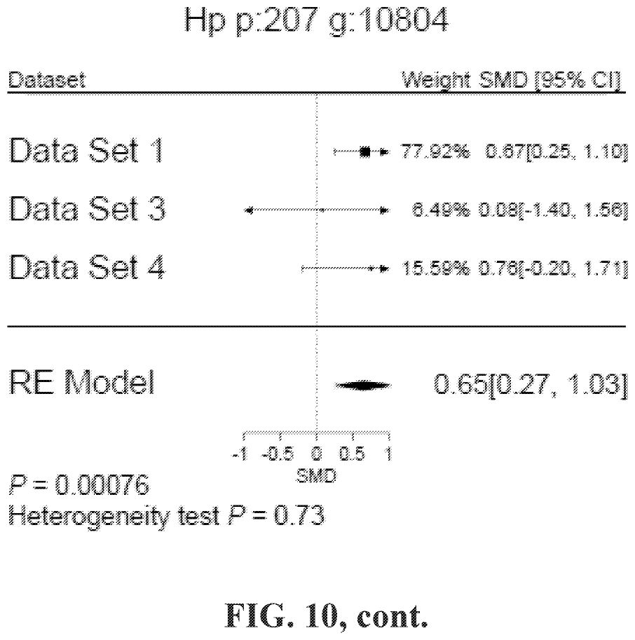

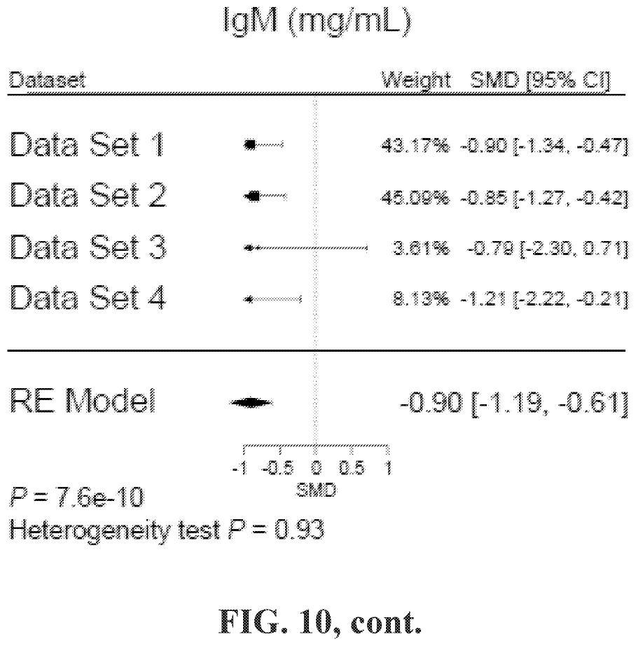

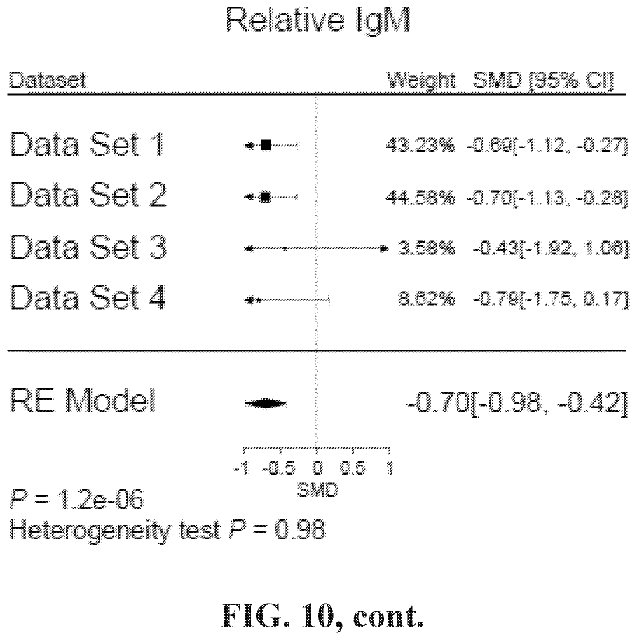

Many biological processes are altered by gender and, ultimately, this leads to differences in disease frequencies and treatment outcomes (29,30). Thus, characterizing gender-specific alterations in glycosylation is an important step in developing glycans as biomarkers of human disease. FIG. 7B reveals that 13 glycopeptides are significantly altered by gender (FDR<0.05), as were the concentrations of the serum proteins A2HSG, A2MG, and IgM (FIG. 7B and Table 3). To confirm these results and the age-glycan associations just described above, a meta-analysis of 4 healthy control datasets was conducted, which confirmed the observed glycan associations across multiple datasets (FIG. 9 and FIG. 10).

### Example 3. Prediction Models for Age

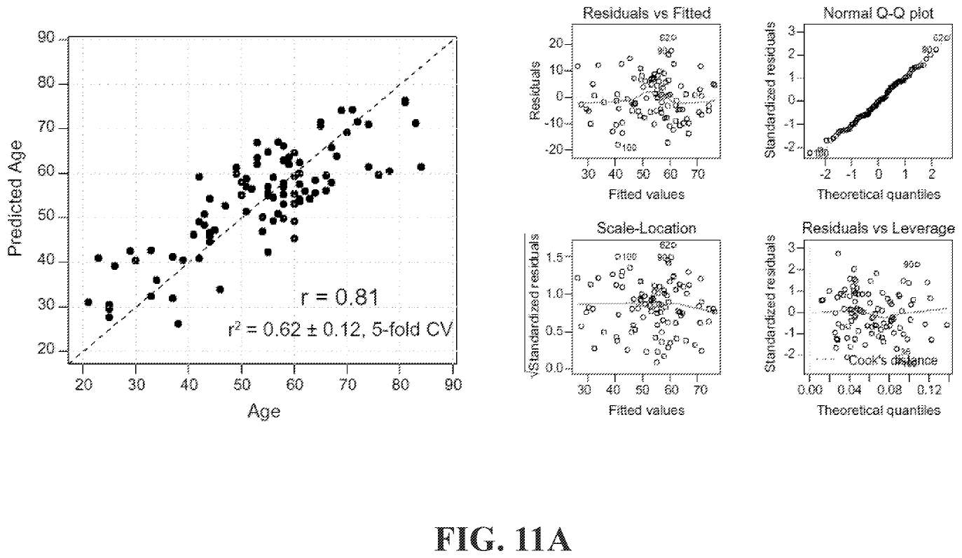

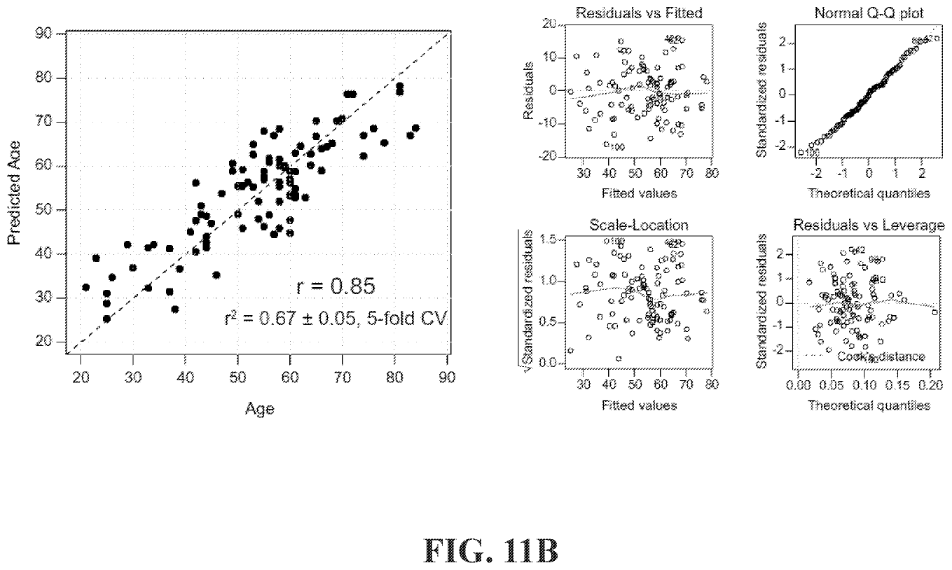

Since there were 41 statistically significant glycopeptides that correlated with age (Table 2), the question arose whether enough information was held within the human glycome to construct an age prediction model. Linear regression models comprised of either glycopeptides only or a mixture of glycopeptides and proteins were thus constructed utilizing a forward stepwise selection method. A resulting “glycan only” model revealed that five sites of glycosylation (IgG1-3510, IgG1-5410, IgM-209-5411, IgM-J-5412, and Haptoglobin (Hp)-241-7602) were sufficient to accurately predict age (PPMCC 0.81) (FIG. 11A and Table 5). Interrogation of the 5-glycopeptide age prediction model revealed low collinearity among its analytes (average variance inflation factor (VIF)=1.34+/−0.19) (Table 5) and the diagnostic plots (residuals vs fitted, normal Q-Q, scale-location, and residuals vs leverage) of the model revealed good linearity, normally distributed residuals, homoscedastic data, and a lack of overly influential cases, respectively (FIG. 11A). The multiple fractional polynomial method (MFP) and individual pairwise PPMCCs were also used to evaluate the model constituents for nonlinear relationships and for correlative relationships amongst each other, respectively. These analyses failed to identify nonlinear relationships or significant intra-model analyte correlations. Thus, all model diagnostics supported the design of the 5-glycopeptide age prediction model. Finally, the age prediction model was successfully validated using a 5-fold cross-validation strategy (r2=0.62+/−0.12, 5-fold CV) (Table 5).

TABLE 5
Exemplary multiple linear regression models for age prediction.
Glycan only model

| | | | | ANCOVA | ANCOVA |
|---|---|---|---|---|---|
| | COEFF | p value | VIF | p value | FDR |
| Intercept | 108.35 | <2e−16 | | | |
| IgG1 g: 3510 | 9.37 | 8.0e−8 | 1.29 | 1.4e−5 | 3.2e−4 |
| IgG1 g: 5410 | −2.82 | 2.4e−5 | 1.51 | 3.2e−9 | 5.9e−7 |
| IgM p: 209 | −257.57 | 1.9e−3 | 1.43 | 7.9e−8 | 7.2e−6 |
| g: 5411 | | | | | |
| IgM J g: 5411 | 23.48 | 1.0e−5 | 1.43 | 5.0e−4 | 5.0e−3 |
| Hp p: 241 | 22.56 | 1.1e−5 | 1.04 | 1.6e−2 | 0.063 |
| g: 7602 | | | | | |

Glycans only model: 5-fold cross validation test performance

| RMSE | R² | RMSE SD | R² SD |
|---|---|---|---|
| 8.65 | 0.62 | 1.16 | 0.12 |

Combined model

| | | | | ANCOVA | ANCOVA |
|---|---|---|---|---|---|
| | COEFF | p value | VIF | p value | FDR |
| Intercept | 82.74 | 1.0e−12 | | | |
| IgG3 Norm | −13.46 | 3.8e−4 | 1.10 | 4.5e−2 | 0.14 |
| Resp | | | | | |
| IgG1 g: 3510 | 5.31 | 8.3e−3 | 2.35 | 1.4e−5 | 3.2e−4 |
| IgG1 g: 5410 | −1.34 | 4.9e−2 | 2.06 | 3.2e−9 | 5.9e−7 |
| IgG2 g: 3410 | 1.69 | 8.3e−4 | 2.29 | 4.5e−7 | 1.6e−5 |
| IgM p: 209 | −335.93 | 2.4e−5 | 1.52 | 7.9e−8 | 7.2e−6 |
| g: 5411 | | | | | |
| IgM J g: 5412 | 27.92 | 5.6e−8 | 1.52 | 5.0e−4 | 5.0e−3 |
| Hp p: 241 | 20.91 | 7.8e−6 | 1.05 | 1.6e−2 | 0.063 |
| g: 7602 | | | | | |

Combined model: 5-fold cross validation test performance

| RMSE | R² | RMSE SD | R² SD |
|---|---|---|---|
| 8.21 | 0.67 | 0.48 | 0.05 |

COEFF: coefficient; VIF: variance inflation factor; ANCOVA: analysis of covariance; FDR: false discovery rate; RMSE: root-mean-square error; R²: coefficient of determination.

Because model constituents IgG1-5410 and IgM-J-5412 had been previously monitored, a meta-analysis was also conducted to determine the weighted averages of their respective glycan-age correlations. These meta-analyses yielded averages that were highly significant (P<2E-16 and P=8.4E-06, respectively) with no evidence (P=0.27 and P=0.93, respectively) of any substantial residual heterogeneity (i.e. there was no remaining variability in effect sizes that was unexplained) (FIG. 9).

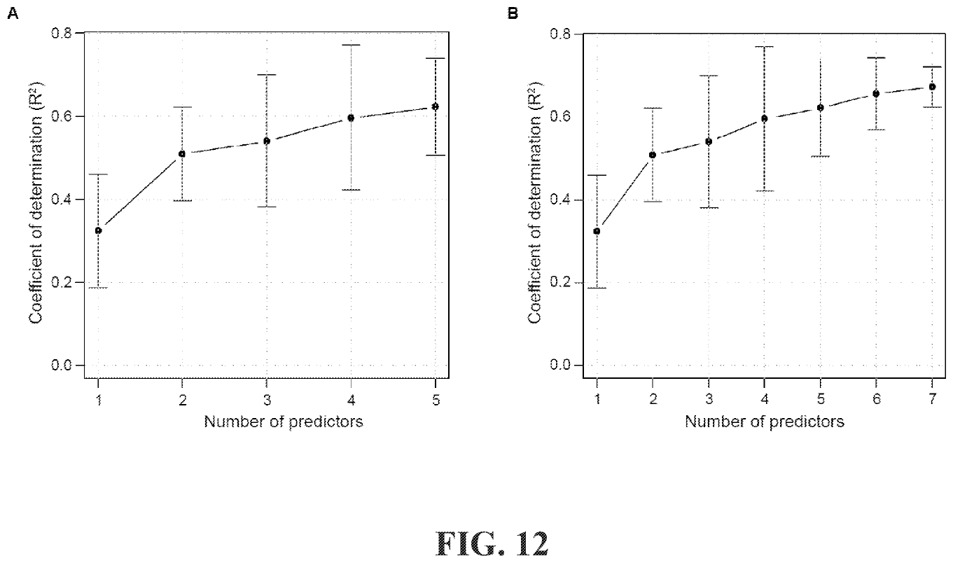

A second combined age-prediction model, which included serum protein concentrations as additional variables, was also constructed. The resulting model contained six glycopeptides (IgG1-3510, IgG1-5410, IgG2-3410, IgM-209-5411, IgM-J-5412, Hp-241-7602) and 1 serum protein (IgG3). This model was also highly accurate in its ability to predict age (PPMCC 0.85; r2=0.67+/−0.05, 5-fold CV) (FIG. 11B) and the diagnostic analyses of this combined model revealed similar results as those just described for the “glycan only” model (FIG. 11B and Table 5). Additional prediction models for age (both “glycan only” and “combined”) with differing numbers of variables were also considered and their summary data are presented in FIG. 12 and Table 6. Of note, in each case the performance of the “glycan only” models were similar to their combined model counterparts, which highlights the utility of glycans as biomarkers of complex biological processes, such as aging.

TABLE 6
Age prediction models with increasing number of predictors.
Glycans only model

| Number of | | | | RMSE | R² |
|---|---|---|---|---|---|
| predictors | Predictors | RMSE | R² | SD | SD |
| 1 | IgG1 g: 5410 | 11.76 | 0.32 | 1.32 | 0.14 |
| 2 | IgG1 g: 3510 + IgG1 g: 5410 | 10.05 | 0.51 | 1.34 | 0.11 |
| 3 | IgG1 g: 3510 + IgG1 g: 5410 + HP | 9.43 | 0.54 | 1.30 | 0.16 |
| | p: 241 g: 7602 | | | | |
| 4 | IgG1 g: 3510 + IgG1 g: 5410 + IgM | 8.70 | 0.60 | 1.76 | 0.17 |
| | J g: 5412 + HP p: 241 g: 7602 | | | | |
| 5 | IgG1 g: 3510 + IgG1 g: 5410 + IgM | 8.65 | 0.62 | 1.16 | 0.12 |
| | p: 209 g: 5411 + IgM J | | | | |
| | g: 5412 + HP p: 241 g: 7602 | | | | |

Combined model

| Number of | | | | RMSE | R² |
|---|---|---|---|---|---|
| predictors | Predictors | RMSE | R² | SD | SD |
| 1 | IgG1 g: 5410 | 11.76 | 0.32 | 1.32 | 0.14 |
| 2 | IgG1 g: 3510 + IgG1 g: 5410 | 10.05 | 0.51 | 1.34 | 0.11 |
| 3 | IgG1 g: 3510 + IgG1 g: 5410 + Hp | 9.43 | 0.54 | 1.30 | 0.16 |
| | p: 241 g: 7602 | | | | |
| 4 | IgG1 g: 3510 + IgG1 g: 5410 + IgM | 8.70 | 0.60 | 1.76 | 0.17 |
| | J g: 5412 + Hp p: 241 g: 7602 | | | | |
| 5 | IgG1 g: 3510 + IgG1 g: 5410 + IgM | 8.65 | 0.62 | 1.16 | 0.12 |
| | p: 209 g: 5411 + IgM J | | | | |
| | g: 5412 + Hp p: 241 g: 7602 | | | | |
| 6 | IgG3 Norm Resp + IgG1 g: 3510 + IgG1 | 8.44 | 0.66 | 0.76 | 0.09 |
| | g: 5410 + IgM p: 209 g: 5411 + IgM | | | | |
| | J g: 5412 + Hp p: 241 g: 7602 | | | | |
| 7 | IgG3 Norm Resp + IgG1 g: 3510 + IgG1 | 8.21 | 0.67 | 0.48 | 0.05 |
| | g: 5410 + IgG2 g: 3410 + IgM p: 209 | | | | |
| | g: 5411 + IgM J g: 5412 + Hp | | | | |
| | p: 241 g: 7602 | | | | |

RMSE: root-mean-square error.
R²: coefficient of determination.

### Example 4. Materials and Methods

Study design. The objective of this study was to identify the relative abundance of site-specific glycosylations within the most abundant plasma proteins and then to use this information to make multianalyte classifiers capable of predicting age. Healthy individuals were recruited from the University of California (UC) Davis Medical Center. The University of California, Davis Institutional Review Board (Committee B) approved this study. Research was performed in accordance with relevant guidelines and regulations. All participants provided their written informed consent.

Sample preparation. For each individual enrolled, plasma was separated from whole blood using a Ficoll gradient. From each plasma preparation, a 2-μL aliquot was reduced, alkylated, and then subjected to trypsin digestion at 37° C. (35). To allow for absolute quantification, 100 μg of IgG, IgA and IgM (all from Sigma-Aldrich, St. Louis, MO) was digested according to the same protocol and a dilution series was made prior to sample injection.

UPLC-ESI-QqQ-MS analysis. The neat enzymatically prepared samples containing both peptides and glycopeptides were then directly analyzed without further hands-on sample cleanup or dilution using an Agilent 1290 infinity liquid chromatography (LC) system coupled to an Agilent 6490 triple quadrupole (QqQ) mass spectrometer (Agilent Technologies, Santa Clara, CA), as previously described (23,35,36). Briefly, an Agilent Eclipse plus C18 (RRHD 1.8 μm, 2.1×100 mm) coupled with an Agilent Eclipse plus C18 pre-column (RRHD 1.8 μm, 2.1×5 mm) was used for UPLC separation. 1.0 μL of the digested plasma samples was injected and analyzed using a 25-minute binary gradient consisting of solvent A of 3% acetonitrile, 0.1% formic acid, solvent B of 90% acetonitrile, 0.1% formic acid in nano-pure water (v/v) at a flow rate of 0.5 mL/min.

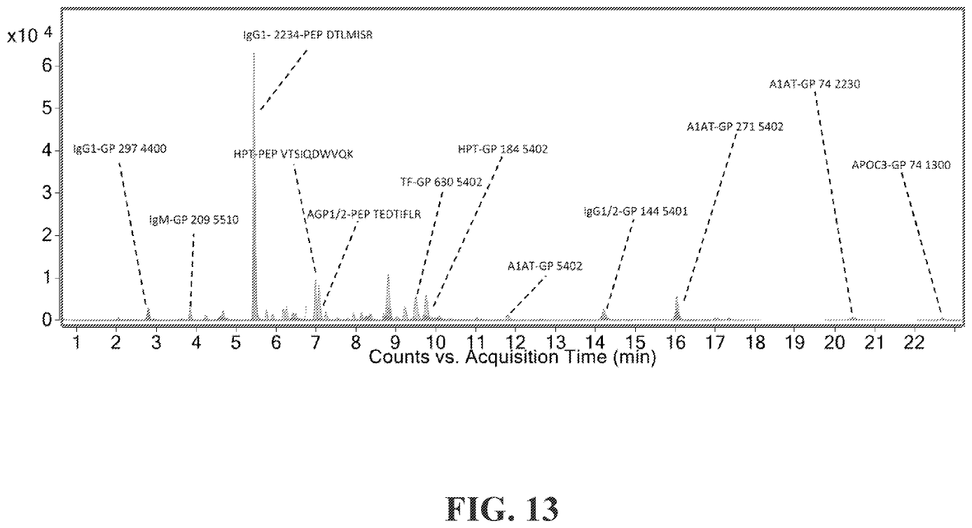

The MRM MS method used for this study requires predetermined knowledge of the peptide or glycopeptide's LC retention time and its collision induced dissociation (CID) behavior, which were previously determined for all the non-glycosylated peptides and glycopeptides used in this study (FIG. 13 and Table 1) (17,35,36). The specific method used herein has been highly validated and the monitored transitions have been described in detail (36). Results were integrated using Agilent MassHunter Quantitative Analysis B.5.0 software. Protein concentrations were determined based on calibration curves and glycopeptide relative responses were calculated using the area under the curves of the glycopeptide and a non-glycosylated reference peptide from the same protein. A list of all analytes monitored in this study is shown in Table 7, and exemplary glycan structures are shown in Table 8.

TABLE 7
List of all analytes monitored.

1) A1AT (mg/mL)
2) A1AT p: 107 g: 5412
3) A1AT p: 107 g: 6503
4) A1AT p: 107 g: 6513
5) A1AT p: 271 g: 5402
6) A1AT p: 271 g: 5412
7) A1AT p: 70 g: 5402
8) A1AT p: 70 g: 5412
9) A2HSG (mg/mL)
10) A2HSG p: 156 g: 5401
11) A2HSG p: 156 g: 5402
12) A2HSG p: 156 g: 5412
13) A2HSG p: 156 g: 5421
14) A2HSG p: 156 g: 6503
15) A2HSG p: 156 g: 6513
16) A2HSG p: 176 g: 5402
17) A2HSG p: 176 g: 5412
18) A2HSG p: 176 g: 5431
19) A2HSG p: 176 g: 6501
20) A2HSG p: 176 g: 7600
21) A2HSG p: 346 g: 1101
22) A2HSG p: 346 g: 2200
23) A2MG (mg/mL)
24) A2MG p: 1424 g: 5401
25) A2MG p: 1424 g: 5402
26) A2MG p: 1424 g: 5411
27) A2MG p: 1424 g: 6511
28) A2MG p: 247 g: 5401
29) A2MG p: 55 g: 5402
30) A2MG p: 55 g: 5412
31) A2MG p: 70 g: 3300
32) A2MG p: 869 g: 5401
33) A2MG p: 991 g: 5402
34) AGP (mg/mL)
35) AGP1 p: 103 8704
36) AGP1 p: 103 g: 6513
37) AGP1 p: 103 g: 7602
38) AGP1 p: 103 g: 7614
39) AGP1 p: 103 g: 7624
40) AGP1 p: 103 g: 9804
41) AGP1 p: 33 g: 5402
42) AGP1 p: 33 g: 6501
43) AGP1 p: 33 g: 6502
44) AGP1 p: 33 g: 6503
45) AGP1 p: 33 g: 6512
46) AGP1 p: 93 g: 6503
47) AGP1 p: 93 g: 6512
48) AGP1 p: 93 g: 6513
49) AGP1 p: 93 g: 7603
50) AGP1 p: 93 g: 7604
51) AGP1 p: 93 g: 7612
52) AGP1 p: 93 g: 7613
53) AGP1 p: 93 g: 8703
54) AGP1 p: 93 g: 8704
55) AGP1/2 p: 56 g: 6502
56) AGP1/2 p: 56 g: 6503
57) AGP1/2 p: 56 g: 6513
58) AGP1/2 p: 72MC g: 6503
59) AGP1/2 p: 72MC g: 6513
60) AGP1/2 p: 72MC g: 7602
61) AGP1/2 p: 72MC g: 7603
62) AGP1/2 p: 72MC g: 7613
63) AGP1/2 p: 72MC g: 7614
64) AGP2 p: 103 g: 6513
65) ApoC3 (mg/mL)
66) ApoC3 p: 74 g: 0300
67) ApoC3 p: 74 g: 1101
68) ApoC3 p: 74 g: 1102
69) ApoC3 p: 74 g: 2211
70) ApoC3 p: 74 g: 2212
71) ApoC3 p: 74 g: 2221
72) ApoC3 p: 74 g: 2230
73) ApoC3 p: 74A.off g: 1101
74) ApoC3 p: 74A.off g: 1102
75) Hp (mg/mL)
76) Hp p: 184 g: 5401
77) Hp p: 184 g: 5402
78) Hp p: 184 g: 5411
79) Hp p: 184 g: 5412
80) Hp p: 184 g: 6501
81) Hp p: 184 g: 6502
82) Hp p: 184 g: 6503
83) Hp p: 184 g: 6512
84) Hp p: 184 g: 6513
85) Hp p: 207 g: 10803
86) Hp p: 207 g: 10804
87) Hp p: 207 g: 11904
88) Hp p: 207 g: 11905
89) Hp p: 207 g: 11914
90) Hp p: 207 g: 11915
91) Hp p: 207 g: 121015
92) Hp p: 241 g: 5401
93) Hp p: 241 g: 5402
94) Hp p: 241 g: 5511
95) Hp p: 241 g: 6501
96) Hp p: 241 g: 6502
97) Hp p: 241 g: 7602
98) Hp p: 241 g: 7604
99) IgA (mg/mL)
100) IgA1 Norm Resp
101) IgA1/2 p: 144 4501
102) IgA1/2 p: 144 g: 4401
103) IgA1/2 p: 144 g: 4500
104) IgA1/2 p: 144 g: 5400
105) IgA1/2 p: 144 g: 5401
106) IgA1/2 p: 144 g: 5402
107) IgA1/2 p: 144 g: 5500
108) IgA1/2 p: 144 g: 5501
109) IgA1/2 p: 144 g: 5502
110) IgA2 Norm Resp
111) IgA2 p: 205 g: 4510
112) IgA2 p: 205 g: 5410
113) IgA2 p: 205 g: 5411
114) IgA2 p: 205 g: 5412
115) IgA2 p: 205 g: 5510
116) IgA2 p: 205 g: 5511
117) IgG (mg/mL)
118) IgG1 g: 3410
119) IgG1 g: 3510
120) IgG1 g: 4400
121) IgG1 g: 4410
122) IgG1 g: 4411
123) IgG1 g: 4500
124) IgG1 g: 4510
125) IgG1 g: 5400
126) IgG1 g: 5410
127) IgG1 g: 5411
128) IgG1 g: 5510
129) IgG1 M.ox Norm Resp
130) IgG1 Ungly
131) IgG1 Ungly Norm Resp
132) IgG1 Norm Resp
133) IgG2 g: 3410
134) IgG2 g: 3510
135) IgG2 g: 4400
136) IgG2 g: 4410
137) IgG2 g: 4411
138) IgG2 g: 4500
139) IgG2 g: 4510
140) IgG2 g: 5411
141) IgG2 g: 5510
142) IgG2 Norm Resp
143) IgG3 Norm Resp
144) IgG3/4 g: 3510
145) IgG3/4 g: 4410
146) IgG3/4 g: 4411
147) IgG3/4 g: 4510
148) IgG4 Norm Resp
149) IgM (mg/mL)
150) IgM p: 205 g: 5512
151) IgM p: 209 g: 4511
152) IgM p: 209 g: 5411
153) IgM p: 209 g: 5412
154) IgM p: 209 g: 5511
155) IgM p: 209 g: 5512
156) IgM p: 439 g: 5200
157) IgM p: 439 g: 6200
158) IgM p: 439 g: 7200
159) IgM p: 439 g: 8200
160) IgM p: 439 g: 9200
161) IgM p: 46 g: 4311
162) IgM p: 46 g: 5411
163) IgM p: 46 g: 5412
164) IgM p: 46 g: 5501
165) IgM p: 46 g: 5502
166) IgM p: 46 g: 5511
167) IgM p: 46 g: 5601
168) IgM J g: 5401
169) IgM J g: 5411
170) IgM J g: 5412
171) Relative IgM
172) IgA1/2
173) IgM p: 439 Ungly
174) IgG3/4
175) TF (mg/mL)
176) TF p: 432 g: 5402
177) TF p: 432 g: 5412
178) TF p: 432 g: 6502
179) TF p: 630 g: 5401
180) TF p: 630 g: 5402
181) TF p: 630 g: 5412
182) TF p: 630 g: 6513
Ungly denotes the lack of a glycan at the conserved CH-2 84.4 glycosylation site of Ig (immunoglobulin).
A.off indicates an ApoC3 variant lacking its terminal alanine.

TABLE 8
Exemplary glycan structures. Blue square: N-acetylglucosamine;
green circle: mannose; yellow circle: galactose; red triangle: fucose;
purple diamond: N-acetylneuraminic acid; yellow square:
N-acetylgalactosamine.

| Composition | Structure |
|---|---|
| 3500 | 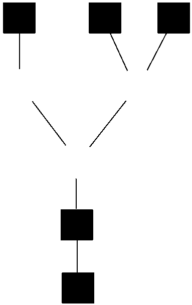 |
| 4401 | 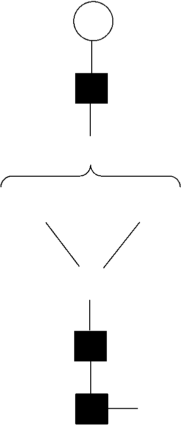 |
| 4500 | 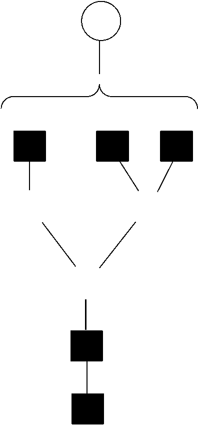 |
| 4501 | 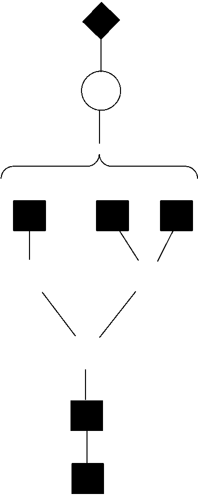 |
| 4510 | 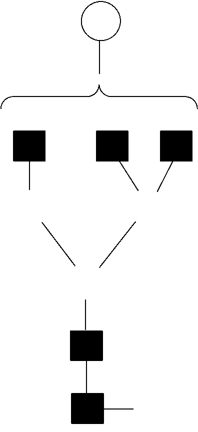 |
| 5200 | 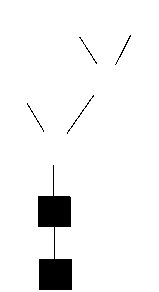 |
| 5400 | 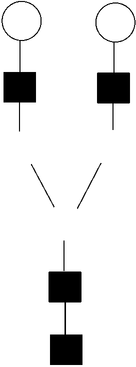 |
| 5401 | 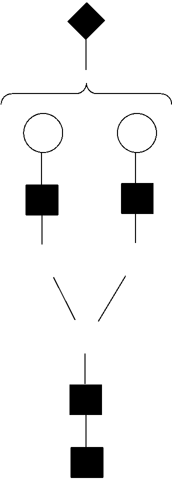 |
| 5402 | 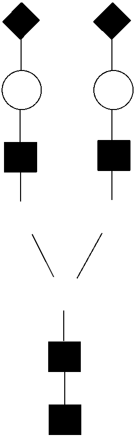 |
| 5410 | 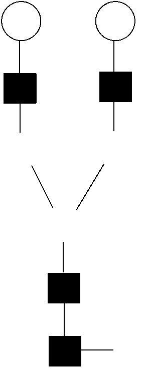 |
| 5411 | 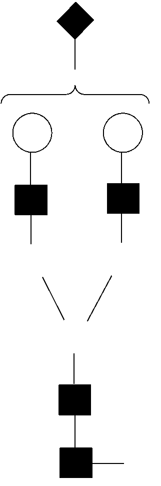 |
| 5412 | 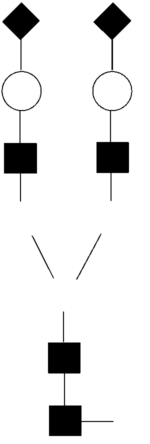 |
| 5500 | 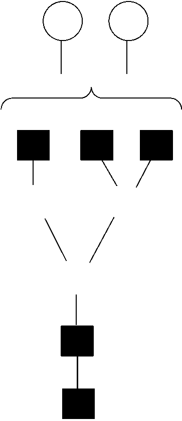 |
| 5501 | 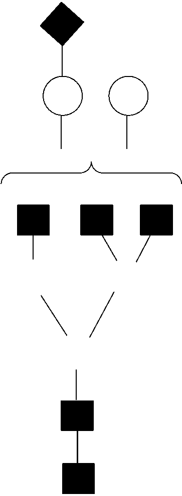 |
| 5502 | 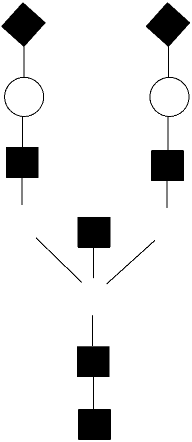 |
| 5510 | 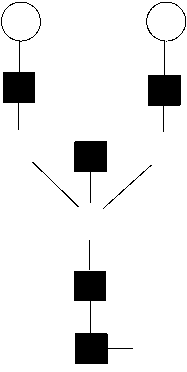 |
| 5511 | 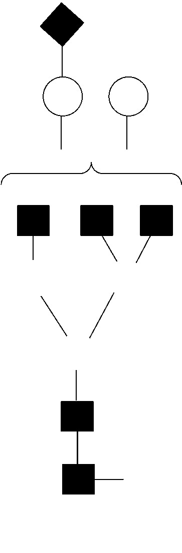 |
| 5512 | 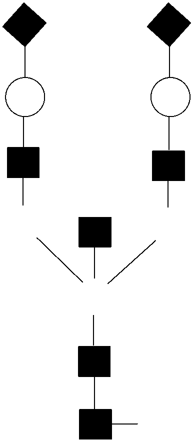 |
| 6200 | 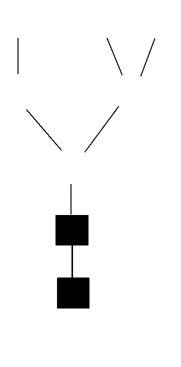 |
| 6501 | 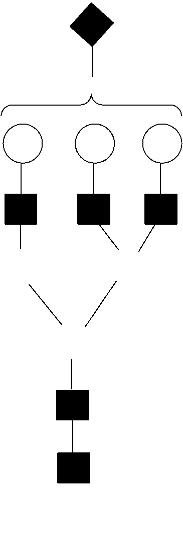 |
| 6502 | 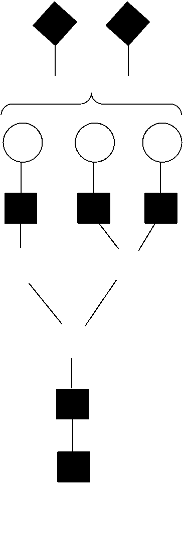 |
| 6503 | 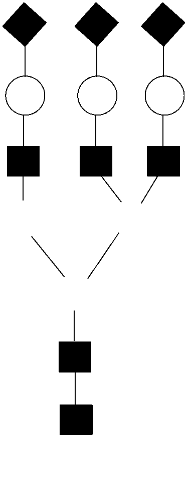 |
| 6512 | 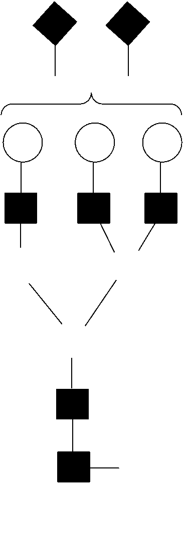 |
| 6513 | 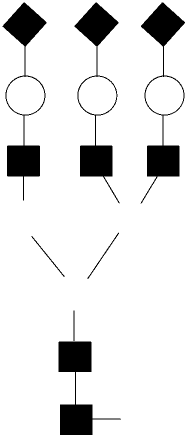 |
| 7200 | 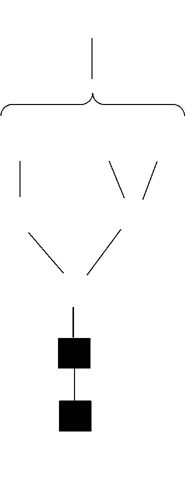 |
| 7602 | 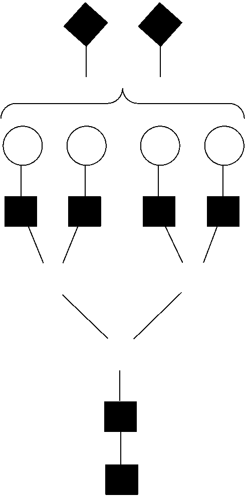 |
| 7603 | 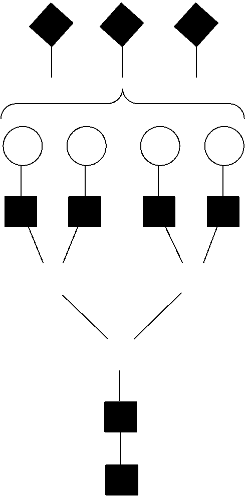 |
| 7604 | 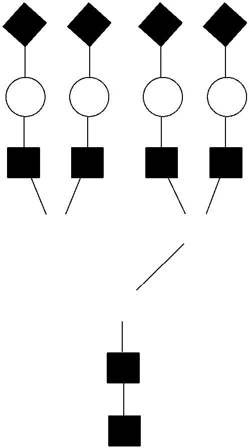 |
| 7613 | 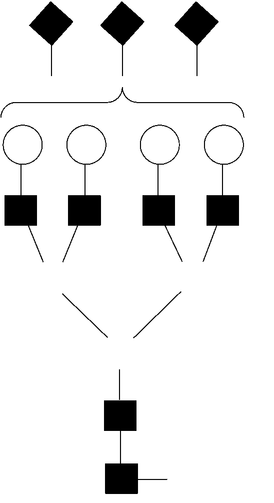 |
| 7614 | 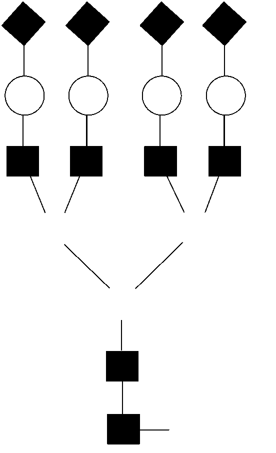 |
| 0300 | 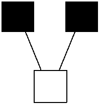 |
| 0310 | 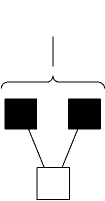 |
| 1101 | 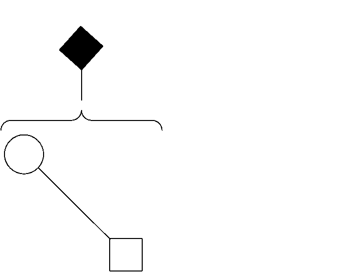 |
| 1102 | 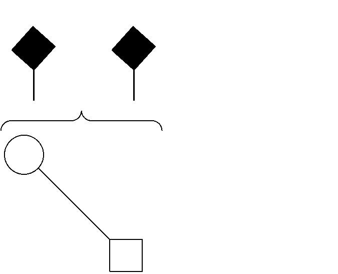 |
| 1111 | 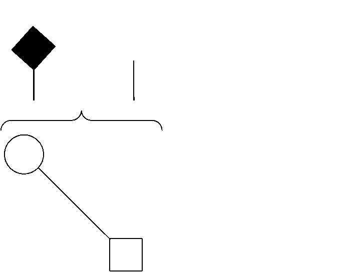 |
| 1201 | 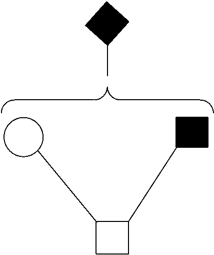 |
| 1202 | 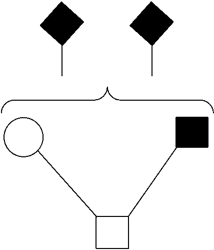 |
| 1210 | 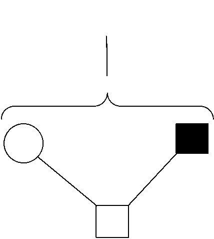 |
| 1300 | 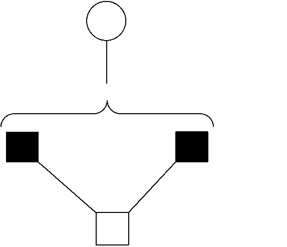 |
| 1311 | 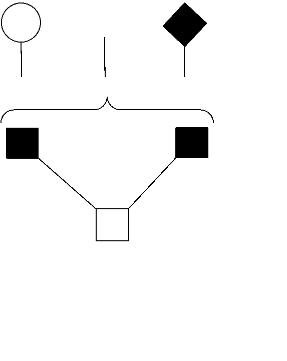 |
| 2200 | 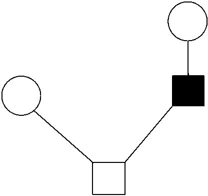 |
| 2211 | 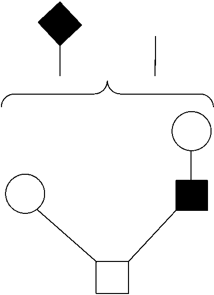 |
| 2212 | 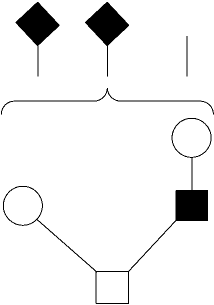 |
| 2220 | 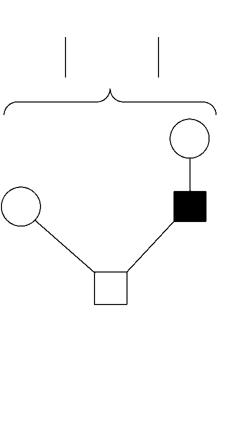 |
| 2221 | 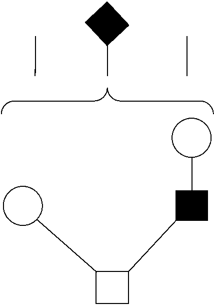 |
| 2230 | 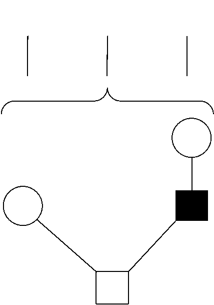 |

Statistical analysis. All statistical analyses were done using R software (37). For each analyte, skewedness was calculated, and data was log transformed when necessary to remove excessive skewness. Outliers were identified using R package “extreamvalues” (38), and when present, were winsorized from the analysis, so that the outliers were set equal to the nearest non-outlier value. Analytes could be detected in all samples; thus, there was no need for imputation of missing data. ANCOVA and linear regression assumptions about the normality of residuals were examined by use of the Shapiro-Wilk test. Colinearity of variables in the multivariate models was examined by calculating variance inflation factor (excessive if >2.5) with R package “car” (39). Nonlinear relationships between the analytes and the outcome were evaluated with R package “mfp” using a multiple fractional polynomial method (40). Variable selection in the multiple linear regressions analyses was performed by forward stepwise exhaustive search using “leaps” R package (41). The algorithm searched the best models of all sizes up to the specified maximum number variables. To identify the best number of variables, each model's performance was estimated by the leave-one-out cross validation method using “caret” (42) R package and the number with minimum root-mean-square error (RMSE) was selected. Logistic regression models were fitted using Firth's bias reduction method with the R package “logistf” (43). This package was also used for automated variable selection based on penalized likelihood ratio tests. Model performance estimated by 5-fold cross-validation was calculated using R package “HandTill2001” (44). Meta-analyses were conducted to assess findings across the multiple datasets using R package “metafor” (45). A weighted random-effects model was used to estimate a summary effect size. Restricted maximum-likelihood estimator was selected to estimate between-study variance. Weighted estimation with inverse-variance weights was used to fit the model. To present the correlations between all analytes simultaneously, the dimensionality reduction algorithm “t-distributed stochastic neighbor embedding” (t-sne) was used, implemented in the R package “Rtsne” (46).

### Example 5. Discussion

Described herein, e.g., in Examples 1-4, is a detailed site-specific map of the human serum glycome, which reveals many novel features of glycosylation. In some cases, glycosylation varied with protein abundance, such that the probability of a particular site-specific glycosylation occurring became rare as the serum concentration of the protein increased (FIG. 5F). Without being bound by theory, this phenomenon may be due to asialoglycoprotein receptor recognition of aged non-sialylated proteins. However, the data described in Examples 1-4 also revealed examples of sialylated glycans negatively correlating with serum protein concentrations (FIG. 5F). Without being bound by theory, this suggests that multiple mechanisms might target a serum protein for clearance, each serving a different purpose. For example, mechanisms to remove aged glycoproteins are clearly needed, and these may be reliant upon non-sialylated proteins being recognized by asialoglycoprotein receptors. However, other scenarios might also impact a glycoprotein's half-life. Theoretically, when an infection resolves, inflammatory mediators should be removed from the circulation. Alternatively, some diseases might negatively impact glycoprotein production. Perhaps there are compensatory mechanisms for low protein production, i.e. increased glycoprotein half-life through altered glycosylation. Of course, the opposite may also be true, disease-related glycan alterations may pathologically signal for the premature clearance of a glycoprotein. The results herein demonstrate that a variety of site-specific glycosylations are associated with glycoprotein serum concentration. It is possible that site-specific glycosylations can fine-tune the plasma half-life of proteins, i.e., that glycoprotein half-life is not merely mediated by age-associated loss of sialylations.

Other interesting phenomena that came to light from the experiments described herein include the observed correlations of site-specific glycosylations across different proteins. This was especially true for IgG1 and IgG2 glycosylations (FIG. 5F). Evidently, there are global signals that help establish the modifying glycans utilized by different B cell populations (those secreting IgG1 and those secreting IgG2). Likewise, several site-specific glycosylations of unrelated proteins were also found to significantly correlate with one another (FIG. 6). However, the strongest site-specific glycan-glycan correlations were generally within the same protein (FIG. 5). Interestingly, not all glycans occurring at a particular site of glycosylation correlated with one another. Thus, the abundance of some glycans did not influence the abundance of others occurring at the exact same site. Perhaps, different influences dictate the abundance of the non-correlating site-specific glycosylations. Alternatively, the same glycoprotein might be synthesized by different cells or subpopulations of cells, each with their own glycosylation signature. Regardless, it is clear that multiple glycosylation influences are applied to glycosylate the same glycosite.

Importantly, the MRM MS method described in the Examples herein is substantially different from methods previously employed for analysis of serum IgG glycans (31,32). Specifically, the prior methods required purification of IgG and enzymatic release of the modifying glycans. In contrast, the method described herein was site-specific and required no protein purification. Thus, the glycan mapping results herein differ from those previously reported (31,32). Furthermore, some amount of glycan structural information is inevitably lost during the ionization process. Thus, different ionization and analysis methods will yield different efficiencies of detection for different glycan structures. The methods herein were not used to definitively determine that a certain glycan structure was more prevalent than another at a specific glycosylation site. Rather, they were used to develop a highly precise method of site-specific glycan detection (i.e., a method with high reproducibility; FIG. 9 and FIG. 10). The monitored glycan structures can be reproducibly detected in all samples with exceptional test-retest reliability, allowing for the construction of clinically relevant multi-analyte glycan biomarker models. It also allows direct comparison of how the abundance of a specific glycan at one glycosylation site correlates to the abundance of a glycan at another glycosylation site. This type of analysis is difficult using traditional MS platforms. Highlighting the power of this method, characterized herein are 16,742 plasma glycan correlations (FIG. 6).

Age and gender are the covariants most commonly accounted for in biomarker research and discovery. As an aid for future glycan biomarker discovery research, glycan alterations associated with these common covariants were identified. Analysis of a large control group, representing healthy individuals ages 21 to 84 years old, demonstrated that IgM was negatively correlated with age (FIG. 7A), a finding supported by other investigations (33). In addition, 41 glycopeptides were found to either positively or negatively correlate with age (Table 2). Analysis of the structures of these glycopeptides revealed a positive association between age and a pro-inflammatory glycosylation profile (less sialylated glycans and more G0 glycans) but this was not a hard-fast rule, as G0 glycans (biantennary structures that terminate in N-acetylglucosamine residues) did not uniformly increase with age across all glycosylation sites and there were also a few non-G0 glycans that increased with age. An age prediction model revealed that five sites of glycosylation were sufficient to accurately predict the age of 97 individuals. The exceptional performance of this model to predict age is a testament of how the human plasma glycome is a reflection of human biological processes, in this case, aging. The calculated glycan age may therefore serve as a predictor of one's natural aging rate, which is obviously different between individuals. Future research into understanding how to alter the human glycome might provide new therapeutic avenues to lower systemic inflammation and possibly even slow aging. The age prediction model(s) constructed herein differ dramatically from previous published work on glycan alterations with aging (24-28,34). Previous models were constructed from released glycans; were not validated; and some were constructed from several glycan “groups” (34), rather than a small number of site-specific glycosylations.

The study described herein is unique for a variety of reasons: 1) glycan quantification was site-specific across multiple serum proteins including different Ig classes and subclasses, while previous studies typically focus on characterizing released glycans or glycoprofiled only a few serum proteins (4-16,31,32); 2) the MRM approach eliminated the need for additional protein purification or chemical processing, which allowed for large patient cohorts to be rapidly characterized; 3) the analysis was precise, rapid, and automated for high throughput; 4) it required only 2 μl of serum or plasma and little sample preparation, while current techniques require several mL of blood to quantitate Ig levels; and 5) in addition to total protein quantification, the technique provided the relative abundance of each glycopeptide, making it more suitable for biomarker research and discovery. For these reasons, the use of this approach as a clinical diagnostic tool is very appealing, especially when compared to its more labor-intensive alternatives (4-16,31,32). Glycan analysis may thus be advantageously applied to the diagnosis and management of human diseases, especially diseases of the immune system and cancer.

### REFERENCES CITED IN THIS DISCLOSURE

1 Apweiler, R., Hermjakob, H. & Sharon, N. On the frequency of protein glycosylation, as deduced from analysis of the SWISS-PROT database. Biochim Biophys Acta 1473, 4-8, doi:10.1016/s0304-4165(99) 00165-8 (1999).
2 in Transforming Glycoscience: A Roadmap for the Future The National Academies Collection: Reports funded by National Institutes of Health (2012).
3 Cummings, R. D. The repertoire of glycan determinants in the human glycome. Mol Biosyst 5, 1087-1104, doi:10.1039/b907931a (2009).
4 Parekh, R. B. et al. Association of rheumatoid arthritis and primary osteoarthritis with changes in the glycosylation pattern of total serum IgG. Nature 316, 452-457 (1985).
5 Parekh, R. B. et al. Galactosylation of IgG associated oligosaccharides: reduction in patients with adult and juvenile onset rheumatoid arthritis and relation to disease activity. Lancet 1, 966-969 (1988).
6 Moore, J. S. et al. Increased levels of galactose-deficient IgG in sera of HIV-1-infected individuals. Aids 19, 381-389 (2005).
7 Holland, M. et al. Differential glycosylation of polyclonal IgG, IgG-Fc and IgG-Fab isolated from the sera of patients with ANCA-associated systemic vasculitis. Biochimica et biophysica acta 1760, 669-677, doi:10.1016/j.bbagen.2005.11.021 (2006).
8 Homma, H. et al. Abnormal glycosylation of serum IgG in patients with IgA nephropathy. Clinical and experimental nephrology 10, 180-185, doi:10.1007/s10157-006-0422-y (2006).
9 Saldova, R. et al. Ovarian cancer is associated with changes in glycosylation in both acute-phase proteins and IgG. Glycobiology 17, 1344-1356, doi:10.1093/glycob/cwm100 (2007).
10 Selman, M. H. et al. IgG fc N-glycosylation changes in Lambert-Eaton myasthenic syndrome and myasthenia gravis. Journal of proteome research 10, 143-152, doi:10.1021/pr1004373 (2011).
11 Kodar, K., Stadlmann, J., Klaamas, K., Sergeyev, B. & Kurtenkov, O. Immunoglobulin G Fc N-glycan profiling in patients with gastric cancer by LC-ESI-MS: relation to tumor progression and survival. Glycoconjugate journal 29, 57-66, doi:10.1007/s10719-011-9364-z (2012).
12 Selman, M. H. et al. Changes in antigen-specific IgG1 Fc N-glycosylation upon influenza and tetanus vaccination. Molecular & cellular proteomics: MCP 11, M111 014563, doi:10.1074/mcp.M111.014563 (2012).
13 Ruhaak, L. R. et al. Enrichment strategies in glycomics-based lung cancer biomarker development. Proteomics. Clinical applications, doi:10.1002/prca.201200131 (2013).
14 Parekh, R. et al. A comparative analysis of disease-associated changes in the galactosylation of serum IgG. J Autoimmun 2, 101-114 (1989).
15 Bond, A. et al. A detailed lectin analysis of IgG glycosylation, demonstrating disease specific changes in terminal galactose and N-acetylglucosamine. J Autoimmun 10, 77-85,doi:10.1006/jaut.1996.0104 (1997).
16 Maverakis, E. et al. Glycans in the immune system and The Altered Glycan Theory of Autoimmunity: a critical review. J Autoimmun 57, 1-13, doi:10.1016/j.jaut.2014.12.002(2015).
17 Hong, Q. et al. A Method for Comprehensive Glycosite-Mapping and Direct Quantitation of Serum Glycoproteins. J Proteome Res 14, 5179-5192, doi:10.1021/acs.jproteome.5b00756 (2015).
18 Li, A. C., Alton, D., Bryant, M. S. & Shou, W. Z. Simultaneously quantifying parent drugs and screening for metabolites in plasma pharmacokinetic samples using selected reaction monitoring information-dependent acquisition on a QTrap instrument. Rapid communications in mass spectrometry: RCM 19, 1943-1950, doi:10.1002/rcm.2008 (2005).
19 Xiao, J. F., Zhou, B. & Ressom, H. W. Metabolite identification and quantitation in LC-MS/MS-based metabolomics. Trends in analytical chemistry: TRAC 32, 1-14, doi:10.1016/j.trac.2011.08.009 (2012).
20 Kitteringham, N. R., Jenkins, R. E., Lane, C. S., Elliott, V. L. & Park, B. K. Multiple reaction monitoring for quantitative biomarker analysis in proteomics and metabolomics. Journal of chromatography. B, Analytical technologies in the biomedical and life sciences 877, 1229-1239, doi:10.1016/j.jchromb.2008.11.013 (2009).
21 Gallien, S., Duriez, E. & Domon, B. Selected reaction monitoring applied to proteomics. Journal of mass spectrometry: JMS 46, 298-312, doi:10.1002/jms. 1895 (2011).
22 Ruhaak, L. R. & Lebrilla, C. B. Applications of Multiple Reaction Monitoring to Clinical Glycomics. Chromatographia, doi:10.1007/s10337-014-2783-9 (2015).
23 Miyamoto, S. et al. Multiple Reaction Monitoring for the Quantitation of Serum Protein Glycosylation Profiles: Application to Ovarian Cancer. J Proteome Res 17, 222-233, doi:10.1021/acs.jproteome.7b00541 (2018).
24 Chen, G. et al. Change in IgG1 Fc N-linked glycosylation in human lung cancer: age-and sex-related diagnostic potential. Electrophoresis 34, 2407-2416, doi:10.1002/elps.201200455 (2013).
25 Chen, G. et al. Human IgG Fc-glycosylation profiling reveals associations with age, sex, female sex hormones and thyroid cancer. Journal of proteomics 75, 2824-2834, doi:10.1016/j.jprot.2012.02.001 (2012).
26 Ding, N. et al. Human serum N-glycan profiles are age and sex dependent. Age and ageing 40, 568-575, doi:10.1093/ageing/afr084 (2011).
27 Ruhaak, L. R. et al. Plasma protein N-glycan profiles are associated with calendar age, familial longevity and health. Journal of proteome research 10, 1667-1674, doi:10.1021/pr1009959 (2011).
28 Parekh, R., Roitt, I., Isenberg, D., Dwek, R. & Rademacher, T. Age-related galactosylation of the N-linked oligosaccharides of human serum IgG. The Journal of experimental medicine 167, 1731-1736 (1988).
29 Whitacre, C. C. Sex differences in autoimmune disease. Nat Immunol 2, 777-780,doi:10.1038/ni0901-777 (2001).
30 Siegel, R. L., Miller, K. D. & Jemal, A. Cancer Statistics, 2017. CA Cancer J Clin 67,7-30, doi:10.3322/caac.21387 (2017).
31 Selman, M. H. et al. Fc specific IgG glycosylation profiling by robust nano-reverse phase HPLC-MS using a sheath-flow ESI sprayer interface. J Proteomics 75, 1318-1329,doi:10.1016/j.jprot.2011.11.003 (2012).
32 Huffman, J. E. et al. Comparative performance of four methods for high-throughput glycosylation analysis of immunoglobulin G in genetic and epidemiological research. Mol Cell Proteomics 13, 1598-1610, doi:10.1074/mcp.M113.037465 (2014).
33 Listi, F. et al. A study of serum immunoglobulin levels in elderly persons that provides new insights into B cell immunosenescence. Annals of the New York Academy of Sciences 1089, 487-495, doi:10.1196/annals.1386.013 (2006).
34 Gudelj, I. et al. Estimation of human age using N-glycan profiles from bloodstains. Int J Legal Med 129, 955-961, doi:10.1007/s00414-015-1162-x (2015).

35 Hong, Q., Lebrilla, C. B., Miyamoto, S. & Ruhaak, L. R. Absolute quantitation of immunoglobulin G and its glycoforms using multiple reaction monitoring. Anal Chem 85,8585-8593, doi:10.1021/ac4009995 (2013).
36 Li, Q. et al. Site-Specific Glycosylation Quantitation of 50 Serum Glycoproteins Enhanced by Predictive Glycopeptidomics for Improved Disease Biomarker Discovery. Anal Chem 91, 5433-5445, doi:10.1021/acs.analchem.9b00776 (2019).
37 R Foundation for Statistical Computing, V., Austria. R Development Core Team (2008) R: A language and environment for statistical computing., <http://www.R-project.org.> (2008).
38 van der Loo, M. P. J. Extremevalues, an R package for outlier detection in univariate data. R package version 2.1., CRAN.R-project.org/package& #x003D;extremevalues (2014).
39 Fox, J. & Weisberg, S. An {R} Companion to Applied Regression, Second Edition. Thousand Oaks CA: Sage., socserv.socsci.mcmaster.ca/jfox/Books/Companion (2011).
40 Royston, P. & Altman, D. G. Regression using fractional polynomials of continuous covariates: parsimonious parametric modelling. Appl Statist 43, 429-467 (1994).
41 Lumley, T. & Miller, A. Leaps: Regression Subset Selection. R package version 3.0,CRAN.R-project.org/package=leaps (2017).
42 Kuhn, M. et al. caret: Classification and Regression Training. R package version 6.0-76.,CRAN.R-project.org/package=caret (2017).
43 Heinze, G. & Ploner, M. logistf: Firth's Bias-Reduced Logistic Regression. R package version 1.22, CRAN.R-project.org/package=logistf (2016).
44 Cullmann, A. D. HandTill2001: Multiple Class Area under ROC Curve. R package version 0.2-12., CRAN.R-project.org/package=HandTill2001 (2016).
45 Viechtbauer, W. Conducting meta-analyses in R with the metafor package. Journal of Statistical Software 36, 1-48 (2010).
46 Krijthe, J. H. Rtsne: T-Distributed Stochastic Neighbor Embedding using a Barnes-Hut Implementation., github.com/jkrijthe/Rtsne (2015).

What is claimed is:

1. A method for determining the age of a biological sample from a subject, the method comprising measuring a relative abundance of at least one glycopeptide in the biological sample and comparing the relative abundance of the at least one glycopeptide to an age prediction model, wherein the age prediction model comprises the relative abundance of the at least one glycopeptide in at least one control biological sample, wherein each control biological sample is from a control individual of a known age, thereby determining the age of the biological sample.

2. The method of claim 1, wherein the age of the subject is determined based on the age of the biological sample.

3. The method of claim 1 or 2, wherein the at least one glycopeptide comprises any of the glycopeptides in Table 2.

4. The method of any one of claims 1-3, wherein the at least one glycopeptide comprises IgG1-3510, IgG1-5410, IgM-209-5411, IgM-J-5412, Haptoglobin (Hp)-241-7602, or a combination thereof.

5. The method of any one of claims 1-4, wherein the at least one glycopeptide comprises IgG1-3510, IgG1-5410, IgM-209-5411, IgM-J-5412, and Haptoglobin (Hp)-241-7602.

6. The method of any one of claims 1-5, wherein the method further comprises measuring a concentration of at least one protein in the biological sample and comparing the concentration of the at least one protein to the age prediction model, and wherein the age prediction model further comprises the concentration of the at least one protein in the at least one control biological sample.

7. The method of claim 6, wherein the at least one protein comprises any of the proteins in Table 2.

8. The method of claim 6 or 7, wherein the at least one protein comprises IgG3.

9. The method of claim 8, wherein the at least one glycopeptide comprises IgG1-3510, IgG1-5410, IgG2-3410, IgM-209-5411, IgM-J-5412, Hp-241-7602, or a combination thereof.

10. The method of claim 8 or 9, wherein the at least one glycopeptide comprises IgG1-3510, IgG1-5410, IgG2-3410, IgM-209-5411, IgM-J-5412, and Hp-241-7602.

11. The method of any one of claims 1-10, wherein the age prediction model comprises the relative abundance of the at least one glycopeptide in a plurality of control biological samples.

12. The method of any one of claims 1-11, wherein the biological sample and the control biological sample are liquid samples.

13. The method of any one of claims 1-12, wherein the biological sample and the control biological sample are blood samples, serum samples, plasma samples, or a combination thereof.

14. The method of any one of claims 1-13, wherein measuring the relative abundance of the at least one glycopeptide comprises mass spectrometry.

15. The method of any one of claims 1-14, wherein measuring the relative abundance of the at least one glycopeptide comprises multiple reaction monitoring mass spectrometry.

16. The method of claim 15, wherein measuring the relative abundance of the at least one glycopeptide comprises calculating the relative response of the at least one glycopeptide as the area under the mass spectrometry curve of the at least one glycopeptide divided by the area under the curve of a non-glycosylated reference peptide from the same protein as the at least one glycopeptide.

17. The method of any one of claims 1-16, wherein the age prediction model comprises a linear regression model or a multiple linear regression model based on a correlation between the relative abundance of the at least one glycopeptide in the at least one control biological sample and the age of the control individual.

18. The method of claim 17, wherein the age prediction model comprises one of the multiple linear regression models of Table 5.

19. The method of any one of claims 1-18, wherein the subject is male or female.

20. The method of any one of claims 1-19, wherein the biological sample is from a criminal forensics investigation.
